(19) United States
(12) Patent Application Publication
(10) Pub. No.: US 2022/0288065 A1
(43) Pub. Date: Sep. 15, 2022

(54) MONOCYCLIC AGONISTS OF STIMULATOR OF INTERFERON GENES STING

(71) Applicant: The Scripps Research Institute, La Jolla, CA (US)

(72) Inventors: H. Michael PETRASSI, San Diego, CA (US); Chenguang YU, San Diego, CA (US); Jie WANG, Shanghai (CN); Arnab K. CHATTERJEE, San Diego, CA (US); Ana Maria Gamo ALBERO, San Diego, CA (US); Anil GUPTA, San Diego, CA (US); Junko TAMIYA, Carlsbad, CA (US); Peter G. SCHULTZ, La Jolla, CA (US); Kristen JOHNSON, Santee, CA (US); Alan CHU, San Diego, CA (US); Emily N. CHIN, San Diego, CA (US); Luke L. LAIRSON, San Diego, CA (US)

(21) Appl. No.: 17/753,138

(22) PCT Filed: Aug. 21, 2020
(86) PCT No.: PCT/US2020/070443

§ 371 Date: Feb. 21, 2022

Related U.S. Application Data
(60) Provisional application No. 62/889,669, filed on Aug. 21, 2019.

(51) Int. Cl. A61K 31/501; A61K 39/395; A61K 45/06; C07D 401/04; C07D 403/04; C07D 413/14; C07D 237/24; C07D 213/82; C07D 401/14; C07D 403/14; C07D 417/04; C07D 413/04; C07D 409/14; C07D 498/04; C07D 239/28; A61K 31/497; A61K 31/506; A61K 31/50; A61K 31/4192; A61K 31/44; A61K 31/536; A61K 31/5365

(52) U.S. Cl. CPC A61K 31/501; A61K 39/3955; A61K 45/06; C07D 401/04; C07D 403/04; C07D 413/14; C07D 237/24; C07D 213/82; C07D 401/14; C07D 403/14; C07D 417/04; C07D 413/04; C07D 409/14; C07D 498/04; C07D 239/28; A61K 31/497; A61K 31/506; A61K 31/50; A61K 31/4192; A61K 31/44; A61K 31/536; A61K 31/5365

(57) ABSTRACT

The invention provides compounds having STimulator of INterferon Genes (STING) agonistic bioactivity that can be used in the treatment of tumors inpatients afflicted therewith. The compounds are of formula (IA), formula (I), and formula (II): wherein the various substituents are as defined herein. Ring A is a 5- or 6-membered heteroaryl comprising 1, 2, or 3 N atoms, unsubstituted or substituted with 1, 2, or 3 groups as defined herein. Compounds for practice of a method of the invention can be delivered via oral delivery for systemic exposure, as well as delivered intratumorally. Antitumor therapy using a compound of formula (I) can further comprise administration of an effective dose of an immunecheckpoint targeting drug.

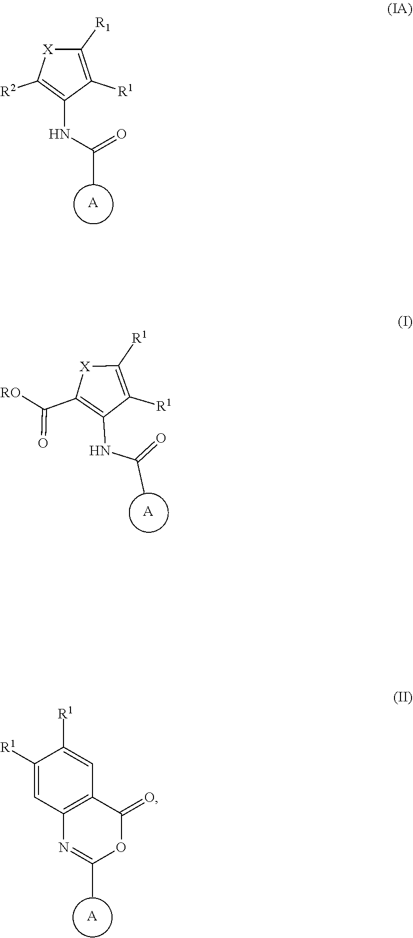

This application claims the benefit of priority to U.S. Patent Application No. 62/889,669 filed on Aug. 21, 2019.

### BACKGROUND

The cGAS-STING signaling pathway plays a critical role in the innate immune response that mammalian host cells mount to eliminate diverse DNA and RNA viruses. STING (Stimulator of Interferon Genes) is an endoplasmic reticulum (ER) resident signaling protein, partially localized to mitochondria-associated membranes, which is broadly expressed in both immune and non-immune cell types. In response to cyclic dinucleotides (CDNs), including 2′-3′ cGAMP produced in response to cytosolic DNA by cyclic GMP-AMP synthase (cGAS), STING translocates to the perinuclear region where it rapidly induces type I interferon (IFN) and pro-inflammatory cytokine production in a TBK1-/IRF3-dependent fashion. STING has also been found to directly bind cytosolic DNA, although the physiological relevance of direct DNA sensing activity remains to be fully characterized.

Recent work has demonstrated that STING plays essential roles in immune responses to tumor cells. Efficient tumor-initiated T cell priming within the tumor microenvironment requires interferon-beta (IFN-b) production by resident dendritic cells and the expression of IFN-b has been demonstrated to be dependent upon activation of the STING pathway (1). Indeed, intratumoral delivery of nucleotide-based STING agonists have been demonstrated to induce the profound regression of established tumors in syngeneic mouse models (1). In addition, activation of the STING pathway has also been demonstrated to significantly contribute to the anti-tumor effect of radiation, via IFN-b mediated immune response within the irradiated tumor microenvironment.

### SUMMARY

In various embodiments, the present disclosure provides an agonist of the Stimulator of Interferon Genes (STING), which can be used in the treatment of tumors.

The present disclosure provides, in various embodiments a compound of formula (IA) or formula (II), or a pharmaceutically acceptable salt thereof,

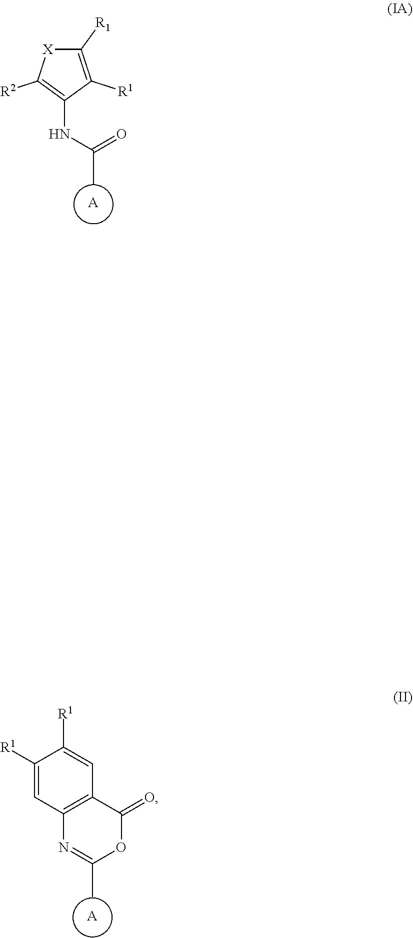

wherein X═S, —N═C(R¹)—, or —C(R¹)═C(R¹).

Each R¹ is independently H, F, Cl, C₁-C₆-alkyl, ethenyl or ethynyl (either of which can be substituted), cyano, alkoxyl, or haloalkyl.

R² is selected from the group consisting of —C(O)OR, —C(O)NH(C₁-C₆-alkyl) (wherein the alkyl is optionally substituted), optionally substituted C₃-C₆-cycloalkenyl, and a 3- to 10-membered heterocyclyl.

R is selected from the group consisting of H, alkyl optionally substituted with —((C₁-C₆-alkyl)OC(O)OC₁-C₆-alkyl) or a 3- to 10-membered heterocyclyl, and benzyl, wherein the benzyl can be unsubstituted or substituted with methoxyl or with an acid or ester isostere.

Ring A is a 5- or 6-membered heteroaryl comprising 1, 2, or 3 N atoms, unsubstituted or substituted with 1, 2, or 3 groups independently selected from the set consisting of NH₂, NH-benzyl unsubstituted or substituted with methoxyl, cyano, alkylnitrile, haloalkyl, hydroxymethyl, aminomethyl, aminopropyl, carboxamido, alkoxy,

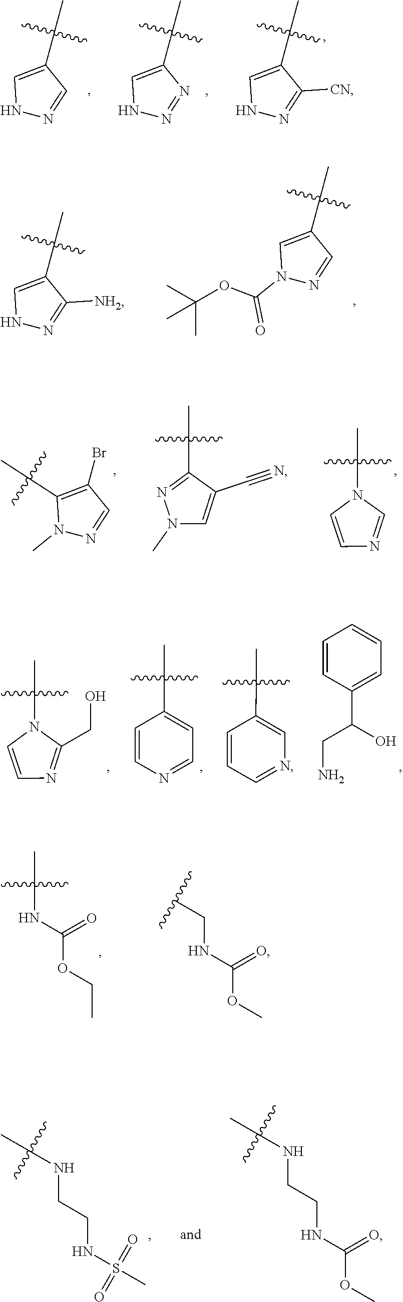

wherein a wavy line indicates a position of bonding.

In various embodiments, the compound of formula (IA) is of formula (I):

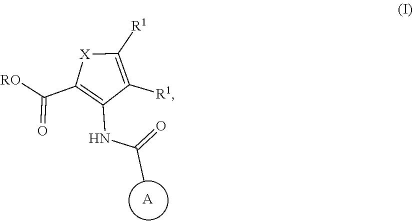

wherein X is S, —N═C(R¹)—, or —C(R¹)═C(R¹)—; R¹=each independently H, F, Cl, ethenyl or ethynyl (either of which can be substituted), cyano, alkoxyl, or haloalkyl; and R is H, alkyl, or benzyl, wherein the benzyl can be unsubstituted or substituted with methoxyl or with an acid or ester isostere such as 1,2,3,4 triazole.

In some embodiments, optionally in combination with any other embodiment described herein, ring A comprises any one of pyridazinyl, triazolyl, pyrimidinyl, or pyridinyl, any of which can be unsubstituted or substituted.

More specifically, per illustrative embodiments, a compound of the present disclosure includes any of the specific compounds shown in Table 1 below.

Further, per an embodiment, the present disclosure provides a method of stimulating expression of interferon genes, comprising administering to a patient an effective dose of an agonist of the Stimulator of Interferon Genes (STING), comprising a compound described herein, and a method of treating a tumor in a patient, comprising administering to the patient an effective dose of an agonist of the Stimulator of Interferon Genes (STING), comprising a compound described herein.

Additionally, a method of the present disclosure can be carried out using an effective dose of any one of the specific compounds disclosed in the application; see, for example, Table 1.

In various embodiments, the method of treatment of a tumor can further comprise administering an effective dose of a compound as disclosed herein via oral or intratumoral administration, or both.

In various embodiments, the method of treatment of a tumor can further comprise administering an effective dose of a compound as disclosed herein, wherein administering comprises administering the compound to the patient as an antibody-drug conjugate, or in a liposomal formulation.

In various embodiments, the method of treatment of a tumor can further comprise administering an effective dose of a compound as disclosed herein, further comprising administration of an effective dose of an immune-checkpoint targeting drug. For example, the immune-checkpoint targeting drug can be an anti-PD-L1 antibody, anti-PD-1 antibody, anti-CTLA-4 antibody, or an anti-4-1BB antibody.

In various embodiments, the method of treatment of a tumor can further comprise administering an effective dose of a compound as disclosed herein, further comprising administration of ionizing radiation or anticancer drugs.

### DETAILED DESCRIPTION

There is significant interest in the development of STING pathway agonists for diverse immuno-oncology applications. Most notably, STING pathway agonists have significant potential application as part of combination therapies involving immune-checkpoint targeting drugs, in patients that fail to respond to checkpoint blockade alone.

We have established a robust platform for identifying non-nucleotide small molecule STING agonists. This has been established using a primary assay involving a human THP-1 cell line carrying an IRF-inducible reporter with 5 copies of the IFN signaling response element. Counter screens, involving alternative reporter constructs, rodent cell-based assays, as well as cGAS and STING knock-out cell lines, are used to eliminate luciferase artifacts and ensure human-rodent cross species reactivity, as well as pathway selectivity. Biochemical assays, involving cGAS enzymatic activity and STING protein binding assays, are used to identify the specific target of identified hits.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the present disclosure refers to an amount of the compound that alleviates, in whole or in part, symptoms associated with the disorder or condition, or halts or slows further progression or worsening of those symptoms, or prevents, or provides prophylaxis for, the disorder or condition. In particular, a “therapeutically effective amount” refers to an amount that is effective, at dosages and for periods of time necessary, to achieve the desired therapeutic result. A therapeutically effective amount is also one in which any toxic or detrimental effects of compounds of the present disclosure are outweighed by the therapeutically beneficial effects.

The expression “effective amount”, when used to describe therapy to an individual suffering from a disorder, refers to the quantity or concentration of a compound of the present disclosure that is effective to inhibit or otherwise act on STING in the individual's tissues wherein STING involved in the disorder, wherein such inhibition or other action occurs to an extent sufficient to produce a beneficial therapeutic effect.

Generally, the initial therapeutically effective amount of a compound described herein or a pharmaceutically acceptable salt thereof that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of the compound or a pharmaceutically acceptable salt thereof. In another embodiment, such dosage forms contain from about 50 mg to about 500 mg of the compound or a pharmaceutically acceptable salt thereof. In yet another embodiment, such dosage forms contain from about 25 mg to about 200 mg of the compound or a pharmaceutically acceptable salt thereof. In still another embodiment, such dosage forms contain from about 10 mg to about 100 mg of the compound or a pharmaceutically acceptable salt thereof. In a further embodiment, such dosage forms contain from about 5 mg to about 50 mg of the compound or a pharmaceutically acceptable salt thereof. In any of the foregoing embodiments the dosage form can be administered once a day or twice per day.

The term “pharmaceutically acceptable salts” refers to nontoxic inorganic or organic acid and/or base addition salts, see, for example, Lit, et al., Salt Selection for Basic Drugs (1986), Int J. Pharm., 33, 201-217, incorporated by reference herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH3)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by the same or differing halogen atoms, such as fluorine and/or chlorine atoms. Examples of haloalkyl include trifluoromethyl, 1,1-dichloroethyl, 1,2-dichloroethyl, 1,3-dibromo-3,3-difluoropropyl, perfluorobutyl, and the like.

Aryl groups are cyclic aromatic hydrocarbons that do not contain heteroatoms in the ring. An aromatic compound, as is well-known in the art, is a multiply-unsaturated cyclic system that contains 4n+2 π electrons where n is an integer. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Heterocyclyl groups or the term “heterocyclyl” includes aromatic and non-aromatic ring compounds containing 3 or more ring members, of which one or more ring atom is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms sums up to equal the total number of ring atoms. Ring sizes can also be expressed by the total number of atoms in the ring, e.g., a 3- to 10-membered heterocyclyl group, counting both carbon and non-carbon ring atoms. A heterocyclyl ring can also include one or more double bonds. A heteroaryl ring is an embodiment of a heterocyclyl group. The term “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The term also includes polycyclic, e.g., bicyclo- and tricyclo-ring systems containing one or more heteroatom such as, but not limited to, quinuclidyl. Heterocyclyl groups can be unsubstituted or can be substituted.

Heteroaryl groups are heterocyclic aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure, which is a multiply-unsaturated cyclic system that contains 4n+2 π electrons wherein n is an integer. A heteroaryl group designated as a C₂-heteroaryl can be a 5-ring (i.e., a 5-membered ring) with two carbon atoms and three heteroatoms, a 6-ring (i.e., a 6-membered ring) with two carbon atoms and four heteroatoms and so forth. Likewise, a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl is also intended to include oxidized S or N, such as sulfonyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

Examples of heteroaryl ring systems described herein include structural unit of formula:

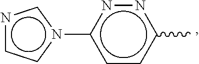

an imidazolyl-pyridazine, which can also be portrayed as:

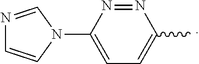

Similarly, other aryl (e.g., phenyl) and heteroaryl (e.g., pyridyl) ring systems described herein can be written either with the explicit double bonds, or with the aryl “circle” nomenclature, but the meanings are the same.

Cycloalkyl groups are groups containing one or more carbocyclic ring including, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

One or more optional substituents on any group described herein are independently selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN. R^{A} and R^{B} are independently selected from the group consisting of H, —CN, —hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S). Each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl). Each alkyl, alkenyl, aryl, and heterocycloalkyl described above is optionally substituted with one or more substituents selected from the group consisting of hydroxy, —OC₁-C₆-alkyl, halo, —NH₂, —(C₁-C₆-alkyl)NH₂, —C(O)OH, CN, and oxo.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

The substituent —CO₂H may be replaced with bioisosteric replacements such as:

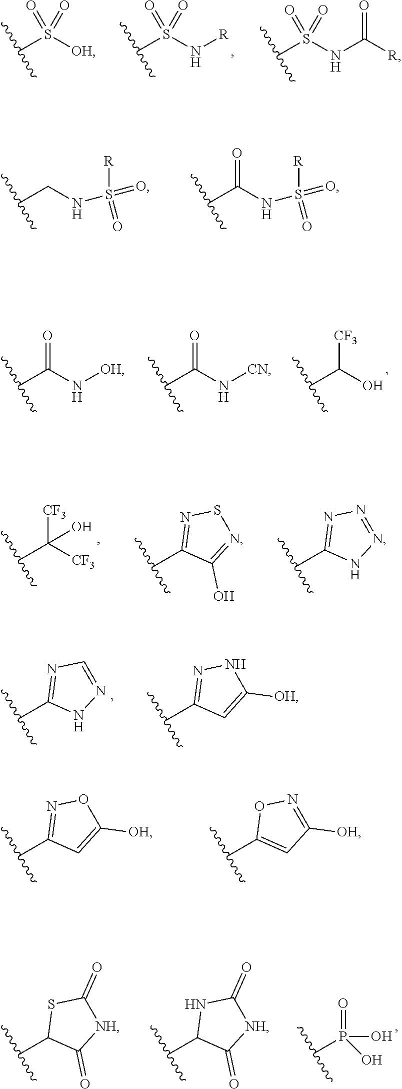

and the like, wherein R has the same definition as R^{A} as defined herein. See, e.g., THE PRACTICE OF MEDICINAL CHEMISTRY (Academic Press: New York, 1996), at page 203.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of formula (I), formula (IA), or formula (II) includes a pharmaceutically acceptable salt of a tautomer of the compound.

Compounds

The present disclosure provides in various embodiments a compound of formula (IA) or formula (II), or a pharmaceutically acceptable salt thereof:

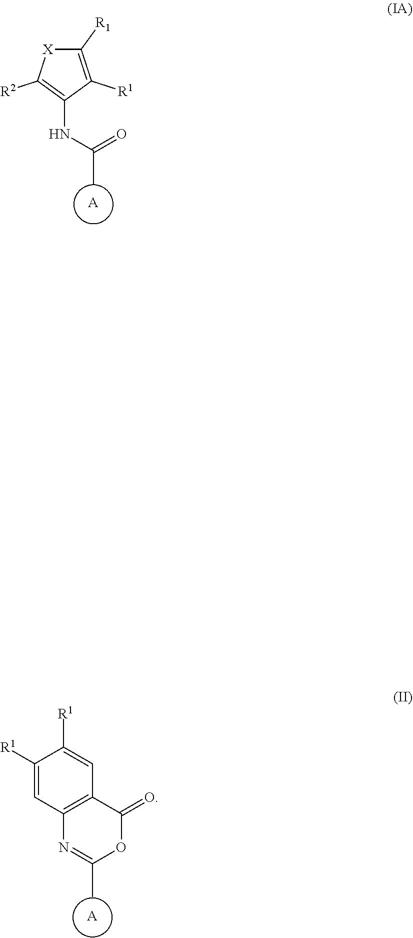

In formula (IA), X is S, —N═C(R¹)—, or —C(R¹)═C(R¹)—.

In some embodiments, the compound is a compound of formula (IA). In other embodiments, the compound is a compound of formula (II).

The present disclosure provides in various embodiments, optionally in combination with any other embodiment described herein, a compound of formula (IA) that is a compound of formula (I) or a pharmaceutically acceptable salt thereof:

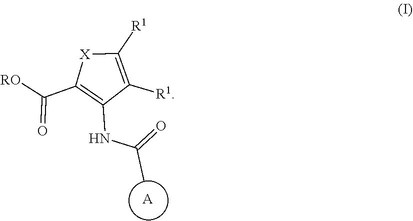

X is S, —N═C(R¹)—, or —C(R¹)═C(R¹)—. Each R¹ is independently H, F, Cl, C₁-C₆-alkyl ethenyl or ethynyl (either of which can be substituted), cyano, alkoxyl, or haloalkyl.

R² is selected from the group consisting of —C(O)OR, —C(O)NH(C₁-C₆-alkyl) (wherein the alkyl is optionally substituted), optionally substituted C₃-C₆-cycloalkenyl, and 3- to 10-membered heterocyclyl. For example, in some embodiments optionally in combination with any other embodiment described herein, R² is —C(O)OR.

R is selected from the group consisting of H, alkyl optionally substituted with —((C₁-C₆-alkyl)OC(O)OC₁-C₆-alkyl) or 3- to 10-membered heterocyclyl, and benzyl, wherein the benzyl can be unsubstituted or substituted with methoxyl or with an acid or ester isostere. In various embodiments, R is H, alkyl, or benzyl, wherein the benzyl can be unsubstituted or substituted with methoxyl or with an acid or ester isostere.

Ring A is a 5- or 6-membered heteroaryl comprising 1, 2, or 3 N atoms, unsubstituted or substituted with 1, 2, or 3 groups independently selected from the group consisting of NH₂, NH-benzyl (wherein the benzyl is unsubstituted or is substituted with methoxyl, cyano, alkylnitrile, haloalkyl, hydroxymethyl, aminomethyl, aminopropyl, carboxamido, or alkoxy),

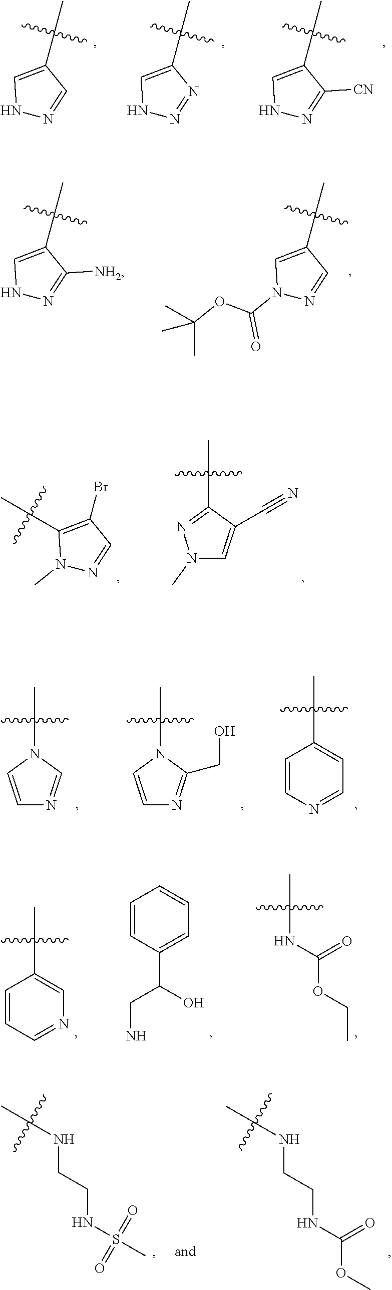

wherein a wavy line indicates a position of bonding.

In various embodiments, ring A comprises any one of pyridazinyl, triazolyl, pyrimidinyl, and pyridinyl, any of which can be unsubstituted or substituted as described herein.

In further embodiments, the present disclosure provides specific examples of compounds, and their pharmaceutically acceptable salts, as set forth in Table 1 below. The compounds are presented with activity scores deriving, in part, from an ISG-LUC activation assay as described herein, and physico-chemical characterizing data.

Table 1: Specific Compounds and Activity Scores. Activity scores are based upon potency and efficacy data (+=EC₅₀>20,000 nM; ++=active but less potent and efficacious than reference compound (EC₅₀>1000 nM); +++=activity comparable to reference compound (EC₅₀<3000 nM); ++++=more potent and/or efficacious than reference compound (EC₅₀<900 nM)).

| | | ISG-LUC | |
|---|---|---|---|
| Compound | | activation | |
| No. | Structure | assay score | Analytical Data |
| 1 | 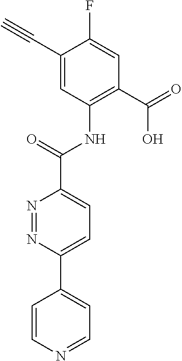 [SMILES: C#Cc1cc(NC(=O)c2ccc(-c3ccncc3)nn2)c(C(=O)O)cc1F] | ++++ | ¹H NMR (400 MHz, DMSO-d₆) δ 15.91 (s, 1H), 8.92 (d, J = 6.5 Hz, 1H), 8.87-8.81 (m, 2H), 8.60 (d, J = 8.8 Hz, 1H), 8.49-8.37 (m, 1H), 8.32- 8.20 (m, 2H), 7.78 (d, J = 10.5 Hz, 1H), 4.54 (s, 1H). MS-ESI: m/z 363.08 observed [M + H]⁺ |
| 2 | 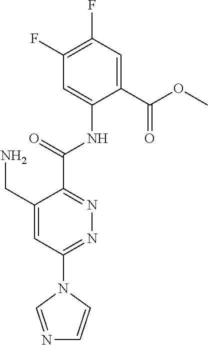 [SMILES: COC(=O)c1cc(F)c(F)cc1NC(=O)c1nnc(-n2ccnc2)cc1CN] | ++ | ¹H NMR (400 MHz, DMSO-d₆) δ 13.11 (s, 1H), 9.43 (s, 1H), 8.91 (s, 1H), 8.83-8.777 (m, 4H), 8.44 (s, 1H), 8.15- 8.10 (m, 1H), 7.62 (s, 1H), 4.68 (t, J = 5.2 Hz, 2H), 3.96 (s, 3H). MS-ESI: m/z 389.22 observed [M + H]⁺ |
| 3 | 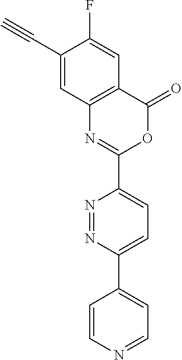 [SMILES: C#Cc1cc2nc(-c3ccc(-c4ccncc4)nn3)oc(=O)c2cc1F] | ++++ | ¹H NMR (400 MHz, DMSO) δ 8.91-8.82 (m, 2H), 8.63 (q, J = 9.0 Hz, 2H), 8.30-8.20 (m, 2H), 8.13 (d, J = 8.6 Hz, 1H), 8.08 (d, J = 6.2 Hz, 1H), 5.04 (s, 1H). MS-ESI: m/z 345.46 observed [M + H]⁺ |
| 4 | 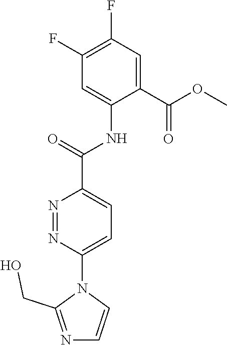 [SMILES: COC(=O)c1cc(F)c(F)cc1NC(=O)c1ccc(-n2ccnc2CO)nn1] | +++ | ¹H NMR(400 MHz, DMSO-d₆) δ 13.05 (s, 1H), 8.85 (dd, J = 13.6, 7.6 Hz, 1H), 8.60 (d, J = 9.2 Hz, 1H), 8.45 (d, J = 9.2 Hz, 1H), 8.11 (dd, J = 11.2, 8.8 Hz, 1H), 7.96 (d, J = 0.8 Hz, 1H), 7.14 (d, J = 0.8 Hz, 1H), 5.55 (t, J = 5.6 Hz, 1H), 4.77 (d, J = 5.6 Hz, 2H), 3.96 (s, 3H). MS-ESI: m/z 390.46 observed [M + H]⁺ |
| 5 | 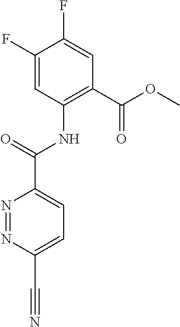 [SMILES: COC(=O)c1cc(F)c(F)cc1NC(=O)c1ccc(C#N)nn1] | ++ | ¹H NMR (400 MHz, DMSO-d₆) δ 13.08 (s, 1H), 8.83-8.78 (m, 1H), 8.64 (q, J = 10.4 Hz, 2H), 8.11 (t, J = 9.2 Hz, 1H), 3.96 (s, 3H). MS-ESI: m/z 319.1 observed [M + H]⁺ |
| 6 | 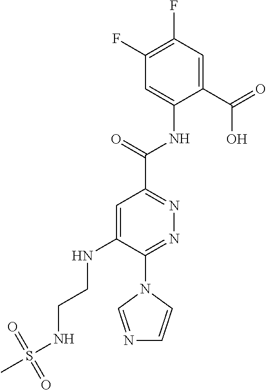 [SMILES: CS(=O)(=O)NCCNc1cc(C(=O)Nc2cc(F)c(F)cc2C(=O)O)nnc1-n1ccnc1] | ++ | ¹H NMR (400 MHz, DMSO-d₆) δ 13.4 (s, 1H), 8.87 (dd, J = 7.2, 13.6 Hz, 1H), 8.17 (s, 1H), 8.06-8.01 (m, 1H), 7.65 (s, 1H), 7.60 (s, 1H), 7.24-7.21 (m, 2H), 6.95 (t, J = 6 Hz, 1H), 3.43-3.41 (m, 2H), 3.38- 3.16 (m, 2H), 2.913 (s, 3H). MS-ESI: m/z 482.42 observed [M + H]⁺ |
| 7 | 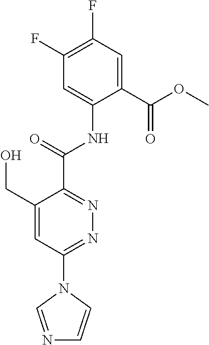 [SMILES: COC(=O)c1cc(F)c(F)cc1NC(=O)c1nnc(-n2ccnc2)cc1CO] | ++++ | ¹H NMR (400 MHz, DMSO-d₆) δ 13.04 (s, 1H), 8.85-8.77 (m, 2H), 8.40 (s, 1H), 8.25-8.24 (m, 1H), 8.09 (dd, J = 9.2, 11.2 Hz, 1H), 7.26 (s, 1H), 5.84 (t, J = 5.2 Hz, 1H), 5.12-5.11 (m, 2H), 3.95 (s, 3H). MS-ESI: m/z 390.23 observed [M + H]⁺ |
| 8 | 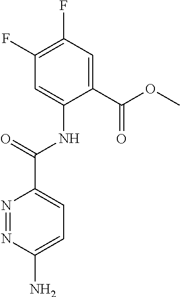 [SMILES: COC(=O)c1cc(F)c(F)cc1NC(=O)c1ccc(N)nn1] | ++ | ¹H NMR (400 MHz, DMSO-d₆) δ 12.72 (s, 1H), 8.83 (dd, J = 14, 7.6 Hz, 1H), 8.07-8.01 (m, 1H), 7.90 (d, J = 9.2 Hz, 1H), 7.204 (s, 2H), 6.91 (d, J = 9.2 Hz, 1H), 3.93 (s, 3H). MS-ESI: m/z 309.17 observed [M + H]⁺ |
| 9 | 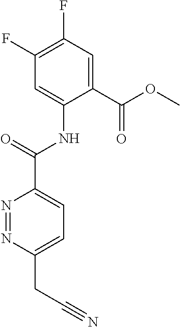 [SMILES: COC(=O)c1cc(F)c(F)cc1NC(=O)c1ccc(CC#N)nn1] | ++ | ¹H NMR (400 MHz, DMSO-d₆) δ 13.08 (s, 1H), 8.83 (dd, J = 7.6, 13.6 Hz, 1H), 8.41 (d, J = 8.8 Hz, 1H), 8.10 (dd, J = 8.8, 11.2 Hz, 1H), 8.01 (d, J = 8.8 Hz, 1H), 4.63 (s, 2H), 3.96 (s, 3H). MS-ESI: m/z 333.14 observed [M + H]⁺ |
| 10 | 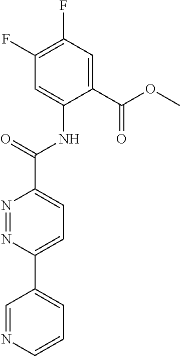 [SMILES: COC(=O)c1cc(F)c(F)cc1NC(=O)c1ccc(-c2cccnc2)nn1] | ++ | ¹H NMR (400 MHz, DMSO-d₆) δ 13.1 (s, 1H), 9.44-9.43 (m, 1H), 8.87 (dd, J = 7.6, 13.6 Hz, 1H), 8.80-8.79 (m, 1H), 8.67-8.63 (m, 2H) 8.47 (d, J = 9.2 Hz, 1H), 8.10 (dd, J = 8.8, 11.2 Hz, 1H), 7.68-7.64 (m, 1H), 3.97 (s, 3H). MS-ESI: m/z 371.27 observed [M + H]⁺ |
| 11 | 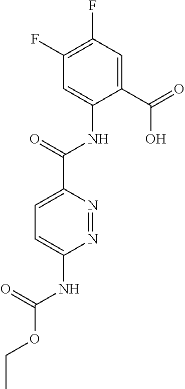 [SMILES: CCOC(=O)Nc1ccc(C(=O)Nc2cc(F)c(F)cc2C(=O)O)nn1] | ++ | ¹H NMR (400 MHz, DMSO-d₆) δ 15.83 (s, 1H), 11.17 (s, 1H), 8.72 (dd, J = 8.14 Hz, 1H), 8.28 (d, J = 9.6 Hz, 1H), 8.24 (d, J = 9.2 Hz, 1H), 7.93-7.87 (m, 1H), 4.22 (q, J = 7.2 Hz, 2H), 1.28 (t, J = 7.2 Hz, 3H). MS-ESI: m/z 367.24 observed [M + H]⁺ |
| 12 | 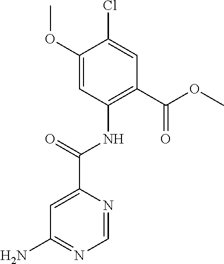 [SMILES: COC(=O)c1cc(Cl)c(OC)cc1NC(=O)c1cc(N)ncn1] | ++ | 1H NMR (400 MHz, DMSO) δ 12.87 (s, 1H), 8.73 (d, J = 2.0 Hz, 1H), 8.55 (s, 1H), 8.02 (d, J = 2.0 Hz, 1H), 7.45 (s, 2H), 7.15 (s, 1H), 3.97 (s, 3H). MS-ESI: m/z 337.44 observed [M + H]⁺ |
| 13 | 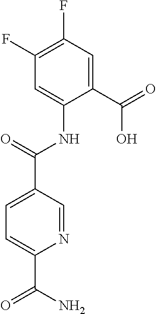 [SMILES: NC(=O)c1ccc(C(=O)Nc2cc(F)c(F)cc2C(=O)O)cn1] | + | ¹H NMR (400 MHz, DMSO-d₆) δ 14.33 (s, 1H), 12.97 (s, 1H), 9.14 (s, 1H), 8.64 (dd, J = 7.6, 13.2 Hz, 1H), 8.45 (dd, J = 1.7, 8, Hz, 1H), 8.30 (s, 1H), 8.22 (d, J = 8.1 Hz, 1H), 8.02 (t, J = 10.8 Hz, 1H). MS-ESI: m/z 322.46 observed [M + H]⁺ |
| 14 | 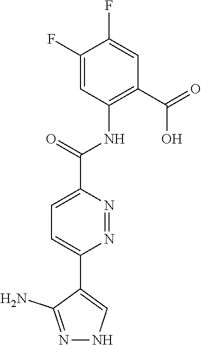 [SMILES: Nc1n[nH]cc1-c1ccc(C(=O)Nc2cc(F)c(F)cc2C(=O)O)nn1] | ++ | MS-ESI: m/z 361.47 observed [M + H]⁺ |
| 15 | 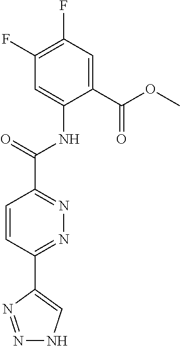 [SMILES: COC(=O)c1cc(F)c(F)cc1NC(=O)c1ccc(-c2c[nH]nn2)nn1] | ++++ | 1H NMR (400 MHz, DMSO-d6) δ 13.17 (s, 1H), 8.88 (q, J = 7.6 Hz, 2H), 8.52-8.45 (m, 2H), 8.13-8.08 (m, 1H), 3.97 (s, 3H). MS-ESI: m/z 360.9 observed [M + H]⁺ |
| 16 | 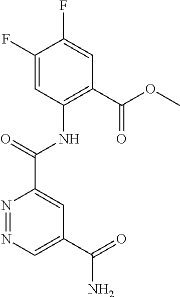 [SMILES: COC(=O)c1cc(F)c(F)cc1NC(=O)c1cc(C(N)=O)cnn1] | ++ | ¹H NMR (400 MHz, DMSO-d₆) δ 13.1 (s, 1H), 9.79 (d, J = 2 Hz, 1H), 8.84 (q, J = 5.6 Hz, 1H), 8.70(d, J = 2 Hz, 2H), 8.14-8.128 (m, 1H), 8.11-8.10 (m, 1H), 3.953 (s, 3H). MS-ESI: m/z 337.3 observed [M + H]⁺ |
| 17 | 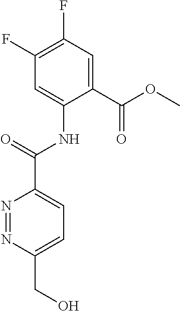 [SMILES: COC(=O)c1cc(F)c(F)cc1NC(=O)c1ccc(CO)nn1] | +++ | ¹H NMR (400 MHz, DMSO-d₆) δ 13.06 (s, 1H), 8.84 (dd, J = 7.6, 13.6 Hz, 1H), 8.40 (d, J = 8.8 Hz, 1H), 8.1 (dd, J = 9.2, 11.2 Hz, 1H), 8.02 (d, J = 8.4 Hz, 1H), 5.85 (t, J = 6 Hz, 1H), 4.91 (d, J = 6 Hz, 2H), 3.95 (s, 3H). MS-ESI: m/z 324.42 observed [M + H]⁺ |
| 18 | 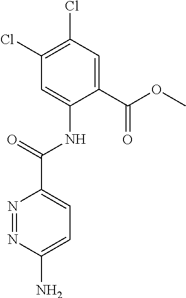 [SMILES: COC(=O)c1cc(Cl)c(Cl)cc1NC(=O)c1ccc(N)nn1] | ++ | ¹H NMR (400 MHz, DMSO-d₆) δ 12.62 (s, 1H), 9.06 (s, 1H), 8.17 (s, 1H), 7.97 (d, J = 9.6 Hz, 1H), 7.59 (s, 2H), 7.06 (d, J = 9.2 Hz, 1H), 3.94 (s, 3H). MS-ESI: m/z 341.41 observed [M + H]⁺ |
| 19 | 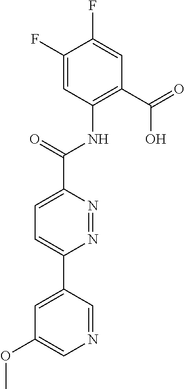 [SMILES: COc1cncc(-c2ccc(C(=O)Nc3cc(F)c(F)cc3C(=O)O)nn2)c1] | ++ | ¹H NMR (400 MHz, DMSO-d₆) δ 15.86 (s, 1H), 9.02 (s, 1H), 8.80- 8.77 (m, 1H), 8.60 (d, J = 8.8 Hz, 1H), 8.50- 8.49 (m, 1H), 8.38 (d, J = 8.8 Hz, 1H), 8.37 (s, 1H), 8.37-7.96 (m, 1H), 3.97 (s, 3H). MS-ESI: m/z 387.0 observed [M + H]⁺ |
| 20 | 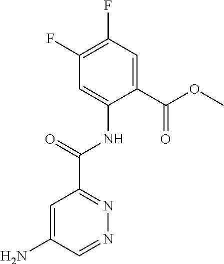 [SMILES: COC(=O)c1cc(F)c(F)cc1NC(=O)c1cc(N)cnn1] | ++++ | ¹H NMR (400 MHz, DMSO-d₆) δ 12.91 (s, 1H), 8.84 (dd, J = 7.6, 13.6 Hz, 1H), 8.69 (d, J = 2.8 Hz, 1H), 8.06 (dd, J = 8.8, 11.2 Hz, 1H), 7.33 (d, J = 2.8 Hz, 1H), 6.91 (s, 2H), 3.94 (s, 3H). MS-ESI: m/z 309.16 observed [M + H]⁺ |
| 21 | 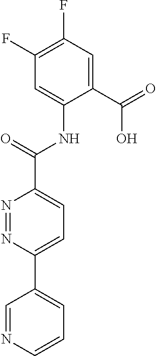 [SMILES: O=C(Nc1cc(F)c(F)cc1C(=O)O)c1ccc(-c2cccnc2)nn1] | ++ | ¹H NMR (400 MHz, DMSO-d6) δ 16.13 (s, 1H), 9.44 (s, 1H), 8.80- 8.74 (m, 2H), 8.66-8.65 (m, 1H), 8.57 (d, J = 8.8 Hz, 1H), 8.39 (d, 8.8 Hz, 1H), 7.96-7.9 (m 1H), 7.68-7.64 (m, 1H). MS-ESI: m/z 357.17 observed [M + H]⁺ |
| 22 | 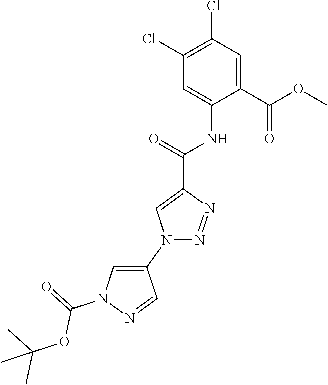 [SMILES: COC(=O)c1cc(Cl)c(Cl)cc1NC(=O)c1cn(-c2cnn(C(=O)OC(C)(C)C)c2)nn1] | ++ | ¹H NMR (400 MHz, CDCl3) δ 12.58 (s, 1H), 9.11 (s, 1H), 8.57 (s, 1H), 8.46 (s, 1H), 8.18 (s, 1H), 8.12 (s, 1H), 4.02 (s, 3H), 1.70 (s, 9H). |
| 23 | 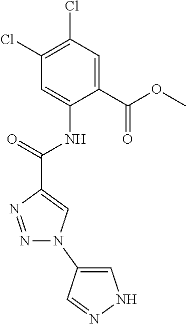 [SMILES: COC(=O)c1cc(Cl)c(Cl)cc1NC(=O)c1cn(-c2cn[nH]c2)nn1] | ++++ | ¹H NMR (400 MHz, DMSO) δ 13.46 (s, 1H), 12.29 (s, 1H), 9.42-9.29 (m, 1H), 9.07-8.94 (m, 1H), 8.32 (s, 2H), 8.22- 8.16 (m, 1H), 7.95 (s, 1H), 3.95 (s, 3H). MS-ESI: m/z 403.36 observed [M + Na]⁺ |
| 24 | 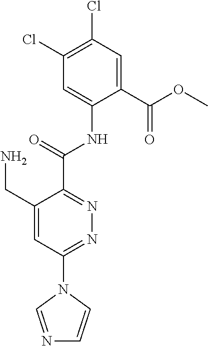 [SMILES: COC(=O)c1cc(Cl)c(Cl)cc1NC(=O)c1nnc(-n2ccnc2)cc1CN] | ++++ | 1H NMR (400 MHz, DMSO-d6) δ 13.06 (s, 1H), 9.06 (s, 2H), 8.72 (s, 1H), 8.60 (s, 3H), 8.29 (s, 1H), 8.25 (s, 1H), 7.45 (s, 1H), 4.64 (d, J = 4.8 Hz, 2H), 3.97 (s, 3H). MS-ESI: m/z 421.4 observed [M + H]⁺ |
| 25 | 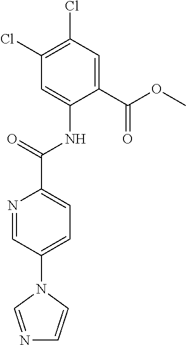 [SMILES: COC(=O)c1cc(Cl)c(Cl)cc1NC(=O)c1ccc(-n2ccnc2)cn1] | ++ | ¹H NMR (400 MHz, DMSO-d₆) δ 12.88 (s, 1H), 9.25 (d, J = 2 Hz, 1H), 9.14 (s, 1H), 8.57 (s, 1H), 8.44-8.41 (m, 1H), 8.31 (d, J = 8.4 Hz, 1H), 8.19 (s, 1H), 8.04- 8.03 (m, 1H), 7.22 (s, 1H), 3.97 (s, 3H). MS-ESI: m/z 391.27 observed [M + H]⁺ |
| 26 | 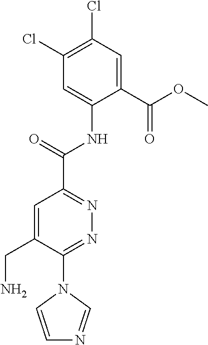 [SMILES: COC(=O)c1cc(Cl)c(Cl)cc1NC(=O)c1cc(CN)c(-n2ccnc2)nn1] | ++++ | ¹H NMR (400 MHz, DMSO-d₆) δ 13.09 (s, 1H), 9.07 (s, 1H), 8.86 (s, 1H), 8.78 (s, 3H), 8.64 (s, 1H), 8.25 (s, 1H), 7.99 (s, 1H), 7.44 (s, 1H), 4.33 (s, 2H), 3.96 (s, 3H). MS-ESI: m/z 421.29 observed [M + H]⁺ |
| 27 | 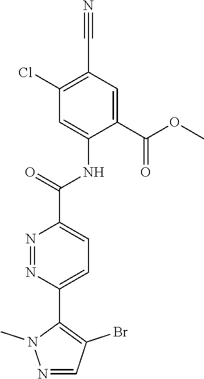 | + | ¹H NMR (400 MHz, DMSO-d₆) δ 13.33 (s, 1H), 9.14 (s, 1H), 8.61- 8.54 (m, 2H), 8.39 (d, J = 8.8 Hz, 1H), 7.86 (s, 1H), 4.06 (s, 3H), 3.99 (s, 3H). MS-ESI: m/z 475.04 observed [M + H]⁺ |
| 28 | 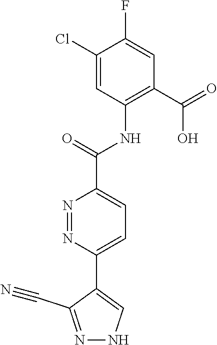 [SMILES: N#Cc1n[nH]cc1-c1ccc(C(=O)Nc2cc(Cl)c(F)cc2C(=O)O)nn1] | ++ | 1H NMR (400 MHz, DMSO-d6) δ 14.46 (s, 1H), 13.61 (s, 1H), 9.04 (d, J = 6.8 Hz, 1H), 8.96-8.94 (m, 1H), 8.44 (d, J = 8.8 Hz, 1H), 8.33-8.30 (m, 1H), 7.99 (d, J = 9.6 Hz, 1H). MS-ESI: m/z 387.0 observed [M + H]⁺ |
| 29 | 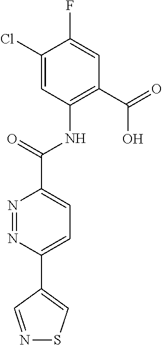 [SMILES: O=C(Nc1cc(Cl)c(F)cc1C(=O)O)c1ccc(-c2cnsc2)nn1] | ++++ | 1H NMR (400 MHz, DMSO-d6) δ 15.67 (s, 1H), 9.95 (s, 1H), 9.42 (s, 1H), 8.97 (d, J = 7.2 Hz, 1H), 8.48 (d, J = 8.8 Hz, 1H), 8.37 (d, J = 8.8 Hz, 1H), 7.92 (s, J = 10.4 Hz, 1H). MS-ESI: m/z 379.0 observed [M + H]⁺ |
| 30 | 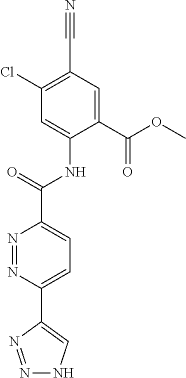 [SMILES: COC(=O)c1cc(C#N)c(Cl)cc1NC(=O)c1ccc(-c2c[nH]nn2)nn1] | ++++ | ¹H NMR (400 MHz, DMSO-d₆) δ 15.77 (s, 1H), 13.37 (s, 1H), 9.13 (s, 1H), 8.87 (s, 1H), 8.59 (s, 1H), 8.51 (d, J = 8.8 Hz, 1H), 8.47 (d, J = 8.8 Hz, 1H), 3.99 (s, 3H). MS-ESI: m/z 384.14 observed [M + H]⁺ |
| 31 | 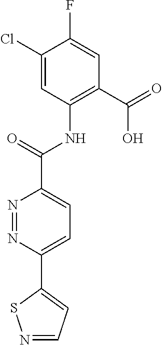 [SMILES: O=C(Nc1cc(Cl)c(F)cc1C(=O)O)c1ccc(-c2ccns2)nn1] | ++++ | 1H NMR (400 MHz, DMSO-d6) δ 15.98 (s, 1H) 8.95 (d, J = 7.2 Hz, 1H), 8.78 (d, J = 1.6 Hz, 1H), 8.57 (d, J = 8.8 Hz, 1H), 8.43 (d, J = 8.8 Hz, 1H), 8.29 (d, J = 2 Hz, 1H), 7.90 (d, J = 10.4 Hz, 1H). MS-ESI: m/z 379.0 observed [M + H]⁺ |
| 32 | 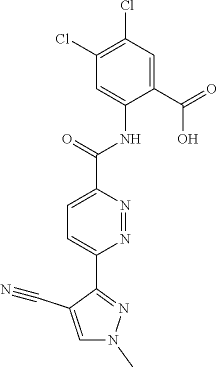 [SMILES: Cn1cc(C#N)c(-c2ccc(C(=O)Nc3cc(Cl)c(Cl)cc3C(=O)O)nn2)n1] | + | ¹H NMR (400 MHz, DMSO-d₆) δ 16.02 (s, 1H), 9.01 (s, 1H), 8.79 (s, 1H), 8.40 (d, J = 8.8 Hz, 1H), 8.35 (d, J = 8.8 Hz, 1H), 8.16 (s, 1H), 4.04 (s, 3H). MS-ESI: m/z 417.12 observed [M + H]⁺ |
| 33 | 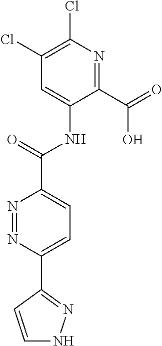 [SMILES: O=C(Nc1cc(Cl)c(Cl)nc1C(=O)O)c1ccc(-c2cc[nH]n2)nn1] | ++ | ¹H NMR (400 MHz, DMSO-d₆) δ 15.54 (s, 1H), 13.473 (s, 1H), 9.33 (s, 1H), 8.38, (d, J = 8.4 Hz, 1H), 8.32 (d, J = 8.8 Hz, 1H), 7.98 (s, 1H), 7.17 (d, J = 2 Hz, 1H). MS-ESI: m/z 379.44 observed [M + H]⁺ |
| 34 | 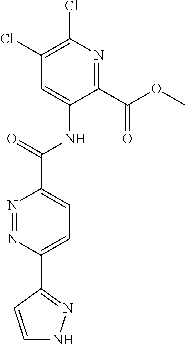 [SMILES: COC(=O)c1nc(Cl)c(Cl)cc1NC(=O)c1ccc(-c2cc[nH]n2)nn1] | ++++ | ¹H NMR (400 MHz, DMSO-d₆) δ 13.52 (s, 1H), 12.97 (s, 1H), 9.43 (s, 1H), 8.46 (d, J = 8.8 Hz, 1H), 8.39 (d, J = 8.8 Hz, 1H), 8.00 (d, J = 2 Hz, 1H), 7.19 (s, 1H), 3.99 (s, 3H). MS-ESI: m/z 393.14 observed [M + H]⁺ |
| 35 | 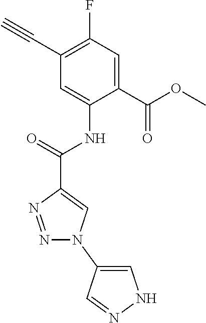 [SMILES: C#Cc1cc(NC(=O)c2cn(-c3cn[nH]c3)nn2)c(C(=O)OC)cc1F] | ++++ | ¹H NMR (400 MHz, DMSO-d₆) δ 13.44 (s, 1H), 12.10 (s, 1H), 9.31 (s, 1H), 8.84 (d, J = 6.4 Hz, 1H), 8.51 (s, 1H), 8.13 (s, 1H), 7.88 (d, J = 9.6 Hz, 1H), 4.85 (s, 1H), 3.95 (s, 3H). MS-ESI: m/z 353.26 observed [M − H]⁺ |
| 36 | 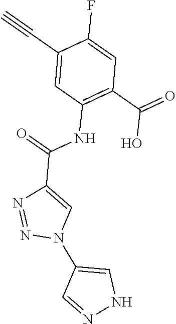 [SMILES: C#Cc1cc(NC(=O)c2cn(-c3cn[nH]c3)nn2)c(C(=O)O)cc1F] | ++ | ¹H NMR (400 MHz, DMSO-d₆) δ 15.39 (s, 1H), 13.40 (s, 1H), 9.11 (d, J = 1.9 Hz, 1H), 8.80 (dd, J = 7.0, 2.0 Hz, 1H), 8.27 (s, 2H), 7.74 (dd, J = 10.6, 2.0 Hz, 1H), 4.52 (d, J = 1.8 Hz, 1H). MS-ESI: m/z 341.06 observed [M + H]⁺ |
| 37 | 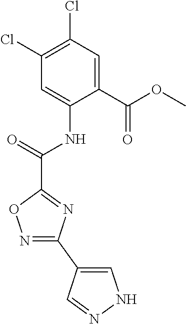 [SMILES: COC(=O)c1cc(Cl)c(Cl)cc1NC(=O)c1nc(-c2cn[nH]c2)no1] | +++ | ¹H NMR (400 MHz, DMSO-d₆) δ 13.60 (s, 1H), 12.24 (s, 1H), 8.75 (s, 1H), 8.50 (s, 1H), 8.21 (s, 1H), 8.07 (s, 1H), 3.97 (s, 3H). MS-ESI: m/z 382.12 observed [M + H]⁺ |
| 38 | 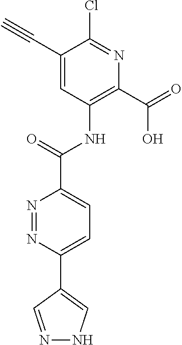 [SMILES: C#Cc1cc(NC(=O)c2ccc(-c3cn[nH]c3)nn2)c(C(=O)O)nc1Cl] | ++++ | ¹H NMR (400 MHz, DMSO-d₆) δ 15.23 (s, 1H), 13 43 (s, 1H), 9.26 (s, 1H), 8.67 (s, 1H), 8.33 (s, 1H), 8.22 (q, J = 7.6 Hz, 2H), 4.80 (s, 1H). MS-ESI: m/z 369.15 observed [M + H]⁺ |
| 39 | 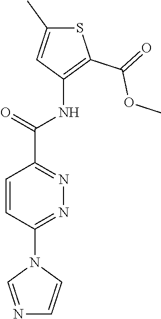 [SMILES: COC(=O)c1sc(C)cc1NC(=O)c1ccc(-n2ccnc2)nn1] | ++ | 1H NMR (400 MHz, DMSO) δ 12.36 (s, 1H), 8.95 (s, 1H), 8.58 (d, J = 9.2 Hz, 1H), 8.49 (d, J = 9.2 Hz, 1H), 8.28 (d, J = 1.9 Hz, 1H), 8.01 (s, 1H), 7.34 (s, 1H), 3.88 (s, 3H), 2.57 (s, 3H). MS-ESI: m/z 344.48 observed [M + H]⁺ |
| 40 | 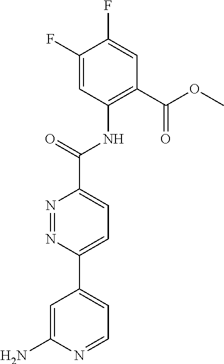 [SMILES: COC(=O)c1cc(F)c(F)cc1NC(=O)c1ccc(-c2ccnc(N)c2)nn1] | ++++ | ¹H NMR (400 MHz, DMSO) δ 13.21 (s, 1H), 8.87 (dd, J = 13.3, 7.5 Hz, 1H), 8.63-8.52 (m, 2H), 8.18 (d, J = 6.3 Hz, 1H), 8.13 (dd, J = 11.2, 8.9 Hz,, 1H), 7.75 (s, 1H), 7.56 (d, J = 6.2 Hz, 1H), 3.97 (s, 3H). MS-ESI: m/z 386.46 observed [M + H]⁺ |
| 41 | 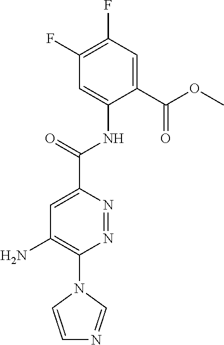 [SMILES: COC(=O)c1cc(F)c(F)cc1NC(=O)c1cc(N)c(-n2ccnc2)nn1] | ++++ | MS-ESI: m/z 375.42 observed [M + H]⁺ |
| 42 | 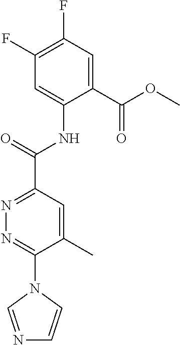 [SMILES: COC(=O)c1cc(F)c(F)cc1NC(=O)c1cc(C)c(-n2ccnc2)nn1] | ++ | ¹H NMR (400 MHz, DMSO) δ 13.08 (s, 1H), 8.85 (dd, J = 7.6, 13.6 Hz, 1H), 8.54 (s, 1H), 8.34 (s, 1H) 8.10 (dd, J = 9.2, 11.2 Hz, 1H), 7.87 (s, 1H), 7.24 (s, 1H), 3.95 (s, 3H), 2.56 (s, 3H). MS-ESI: m/z 374.28 observed [M + H]⁺ |
| 43 | 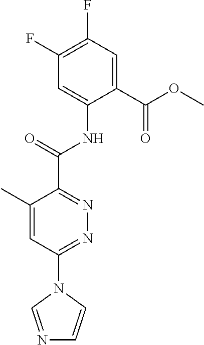 | ++ | ¹H NMR (400 MHz, DMSO) δ 12.96 (s, 1H), 8.49-8.78 (m, 2H), 8.34 (d, J = 0.8 Hz, 1H), 8.18-8.17 (m, 1H), 8.09 (dd, J = 8.8, 11.2 Hz, 1H), 7.27-7.26 (m, 1H), 3.94 (s, 3H), 2.8-2.77 (m, 3H). MS-ESI: m/z 374.25 observed [M + H]⁺ |
| 44 | 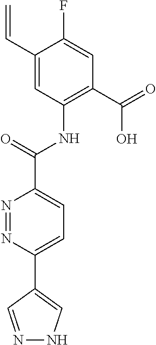 [SMILES: C=Cc1cc(NC(=O)c2ccc(-c3cn[nH]c3)nn2)c(C(=O)O)cc1F] | ++++ | 1H NMR (400 MHz, DMSO-d6) δ 15.61 (s, 1H), 13.44 (s, 1H), 9.01 (d, J = 7.2 Hz, 1H), 8.66 (s, 1H), 8.32 (s, 1H), 8.26-8.15 (m, 2H), 7.70 (d, J = 11.8 Hz, 1H), 6.86 (dd, J = 17.8, 11.1 Hz, 1H), 5.86 (d, J = 17.7 Hz, 1H), 5.47 (d, J = 11.2 Hz, 1H). MS-ESI: m/z 354.53 observed [M + H]⁺ |
| 45 | 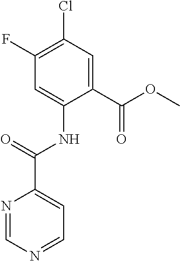 [SMILES: COC(=O)c1cc(Cl)c(F)cc1NC(=O)c1ccncn1] | ++ | 1H NMR (400 MHz, DMSO) δ 12.98 (s, 1H), 9.51-9.50 (m, 1H), 9.20 (d, J = 4.8 Hz, 1H), 8.82 (d, J = 12.4 Hz, 1H), 8.24-8.20 (m, 2H), 3.97 (s, 3H). MS-ESI: m/z 310.43 observed [M + H]⁺ |
| 46 | 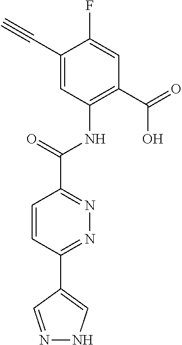 [SMILES: C#Cc1cc(NC(=O)c2ccc(-c3cn[nH]c3)nn2)c(C(=O)O)cc1F] | ++ | 1H NMR (400 MHz, DMSO) δ 15.63 (s, 1H), 8.90 (d, J = 6.5 Hz, 1H), 8.48 (s, 2H), 8.19 (q, J = 8.8 Hz, 2H), 7.76 (d, J = 10.2 Hz, 1H), 4.53 (s, 1H). MS-ESI: m/z 352.48 observed [M + H]⁺ |
| 47 | 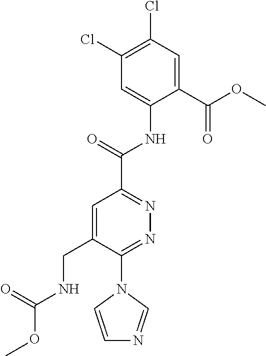 [SMILES: COC(=O)NCc1cc(C(=O)Nc2cc(Cl)c(Cl)cc2C(=O)OC)nnc1-n1ccnc1] | ++ | 1H NMR (400 MHz, DMSO) δ 13.03 (s, 1H), 9.07 (s, 1H), 8.43 (s, 1H), 8.35 (s, 1H), 8.22 (s, 1H), 8.11 (t, J = 5.6 Hz, 1H), 7.89 (s, 1H), 7.27 (s, 1H), 4.42 (d, J = 6 Hz, 2H), 3.96 (s, 3H), 3.58 (s, 3H). MS-ESI: m/z 479.20 observed [M + H]⁺ |
| 48 | 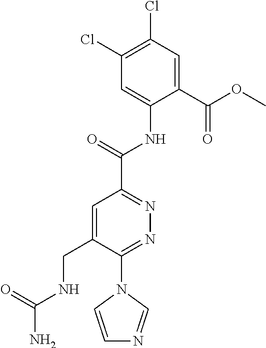 [SMILES: COC(=O)c1cc(Cl)c(Cl)cc1NC(=O)c1cc(CNC(N)=O)c(-n2ccnc2)nn1] | ++ | ¹H NMR (400 MHz, DMSO-d₆) δ 9.89 (s, 1H), 9.09 (s, 1H), 8.56 (s, 1H), 8.44 (t, J = 1.6 Hz, 1H), 8.24 (s, 1H), 8.06 (s, 1H), 4.42 (s, 2H), 3.96 (3H). MS-ESI: m/z 464.26 observed [M + H]⁺ |
| 49 | 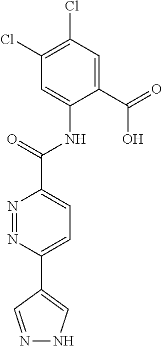 [SMILES: O=C(Nc1cc(Cl)c(Cl)cc1C(=O)O)c1ccc(-c2cn[nH]c2)nn1] | ++ | ¹H NMR (400 MHz, DMSO-d₆) δ 15.75 (s, 1H), 13.51 (s, 1H), 9.01 (s, 1H), 8.50 (s, 1H), 8.25-8.16 (m, 3H), 7.27-7.14 (m, 1H). MS-ESI: m/z 378.41 observed [M + H]⁺ |
| 50 | 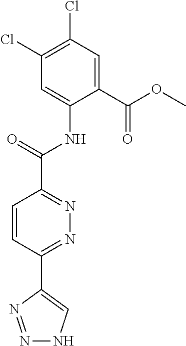 [SMILES: COC(=O)c1cc(Cl)c(Cl)cc1NC(=O)c1ccc(-c2c[nH]nn2)nn1] | ++++ | ¹H NMR (400 MHz, DMSO-d₆) δ 15.75 (s, 1H), 13.10 (s, 1H), 9.10 (s, 1H), 8.88-8.83 (m, 1H), 8.46 (dd, J = 9.2, 21.2 Hz, 2H), 8.20 (s, 1H), 3.97 (s, 3H). MS-ESI: m/z 393.44 observed [M + H]⁺ |
| 51 | 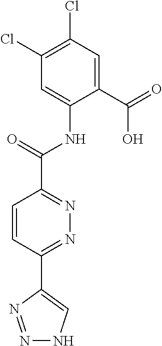 [SMILES: O=C(Nc1cc(Cl)c(Cl)cc1C(=O)O)c1ccc(-c2c[nH]nn2)nn1] | ++ | ¹H NMR (400 MHz, DMSO-d₆) δ 15.24- 15.16 (m, 1H), 9.07 (s, 1H), 8.49-8.40 (m, 3H), 8.20 (s, 1H). MS-ESI: m/z 378.9 observed [M + H]⁺ |
| 52 | 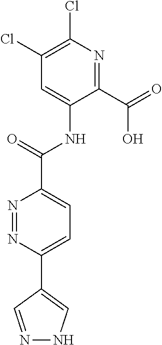 [SMILES: O=C(Nc1cc(Cl)c(Cl)nc1C(=O)O)c1ccc(-c2cn[nH]c2)nn1] | ++++ | ¹H NMR (400 MHz, DMSO-d₆) δ 15.48 (d, J = 3.1 Hz, 1H), 13.44 (s, 1H), 9.31 (d, J = 1.8 Hz, 1H), 8.49 (d, J = 123.8 Hz, 2H), 8.22 (dd, J = 6.3, 2.1 Hz, 2H). MS-ESI: m/z 379.33 observed [M + H]⁺ |
| 53 | 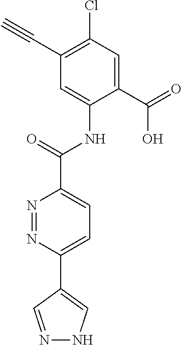 [SMILES: C#Cc1cc(NC(=O)c2ccc(-c3cn[nH]c3)nn2)c(C(=O)O)cc1Cl] | ++ | ¹H NMR (400 MHz, DMSO-d₆) δ 15.61 (s, 1H), 13.50 (s, 1H), 8.98 (s, 1H), 8.51 (s, 1H), 8.20 (J = 8.8, 16.4 Hz, 2H), 8.07 (s, 1H), 4.59 (s, 1H). MS-ESI: m/z 368.1 observed [M + H]⁺ |
| 54 | 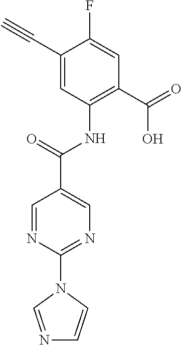 [SMILES: C#Cc1cc(NC(=O)c2cnc(-n3ccnc3)nc2)c(C(=O)O)cc1F] | NA | ¹H NMR (400 MHz, DMSO-d₆) δ 12.90 (s, 1H), 9.46 (s, 2H), 8.87 (s, 1H), 8.77 (d, J = 6.4 Hz, 1H), 8.21 (s, 1H), 8.01 (d, J = 10 Hz, 1H), 7.39 (s, 1H). MS-ESI: m/z 352.2 observed [M + H]⁺ |
| 55 | 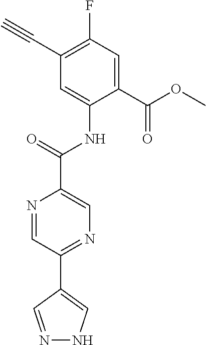 [SMILES: C#Cc1cc(NC(=O)c2cnc(-c3cn[nH]c3)cn2)c(C(=O)OC)cc1F] | + | ¹H NMR (400 MHz, DMSO-d₆) δ 12.62 (s, 1H), 9.23 (d, J = 11.6 Hz, 2H), 8.99 (d, J = 6.4 Hz, 1H), 8.48 (s, 2H), 7.89 (d, J = 10 Hz, 1H), 4.86 (s, 1H), 3.96 (s, 1H). MS-ESI: m/z 366.0 observed [M + H]⁺ |
| 56 | 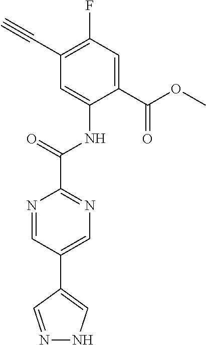 [SMILES: C#Cc1cc(NC(=O)c2ncc(-c3cn[nH]c3)cn2)c(C(=O)OC)cc1F] | + | ¹H NMR (400 MHz, DMSO-d₆) δ 12.84 (s, 1H), 9.39 (s, 2H), 9.03 (d, J = 6.4 Hz, 1H), 8.46 (s, 2H), 7.92 (d, J = 9.6 Hz, 1H), 4.88 (s, 1H), 3.98 (s, 3H). MS-ESI: m/z 366.5 observed [M + H]⁺ |
| 57 | 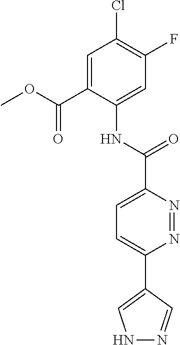 [SMILES: COC(=O)c1cc(Cl)c(F)cc1NC(=O)c1ccc(-c2cn[nH]c2)nn1] | ++++ | MS-ESI: m/z 376.47 observed [M + H]+ |
| 58 | 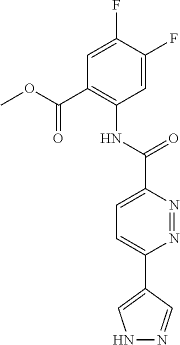 [SMILES: COC(=O)c1cc(F)c(F)cc1NC(=O)c1ccc(-c2cn[nH]c2)nn1] | ++++ | ¹H NMR (400 MHz, DMSO) δ 13.44 (s, 1H), 13.11 (s, 1H), 8.88 (dd, J = 13.6, 7.6 Hz, 1H), 8.71 (s, 1H), 8.35 (s, 1H), 8.32 (d, J = 8.8 Hz, 1H), 8.26 (d, J = 8.8 Hz, 1H), 8.10 (dd, J = 11.3, 9.0 Hz, 1H), 3.96 (s, 3H). MS-ESI: m/z 360.41 observed [M + H]+ |
| 59 | 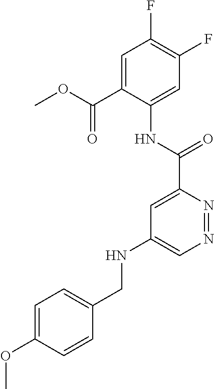 [SMILES: COC(=O)c1cc(F)c(F)cc1NC(=O)c1cc(NCc2ccc(OC)cc2)cnn1] | ++++ | MS-ESI: m/z 429.48 observed [M + H]+ |
| 60 | 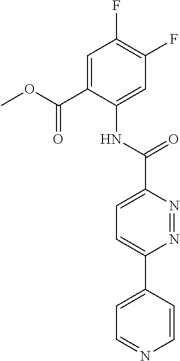 [SMILES: COC(=O)c1cc(F)c(F)cc1NC(=O)c1ccc(-c2ccncc2)nn1] | ++++ | MS-ESI: m/z 371.47 observed [M + H]+ |
| 61 | 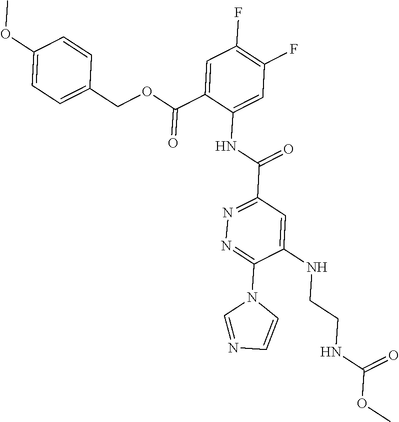 [SMILES: COC(=O)NCCNc1cc(C(=O)Nc2cc(F)c(F)cc2C(=O)OCc2ccc(OC)cc2)nnc1-n1ccnc1] | ++ | MS-ESI: m/z 582.61 observed [M + H]+ |
| 62 | 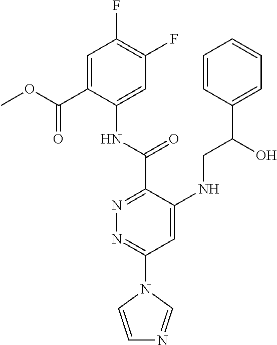 [SMILES: COC(=O)c1cc(F)c(F)cc1NC(=O)c1nnc(-n2ccnc2)cc1NCC(O)c1ccccc1] | ++ | MS-ESI: m/z 395.46 observed [M + H]+ |
| 63 | 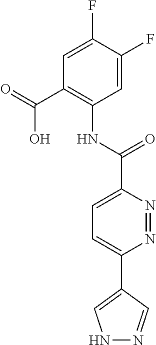 [SMILES: O=C(Nc1cc(F)c(F)cc1C(=O)O)c1ccc(-c2cn[nH]c2)nn1] | ++ | MS-ESI: m/z 346.44 observed [M + H]+ |
| 64 | 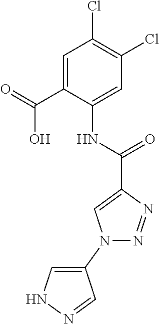 [SMILES: O=C(Nc1cc(Cl)c(Cl)cc1C(=O)O)c1cn(-c2cn[nH]c2)nn1] | ++ | MS-ESI: m/z 366.7 observed [M + H]+ |
| 65 | 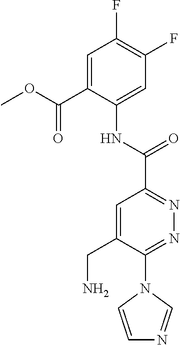 [SMILES: COC(=O)c1cc(F)c(F)cc1NC(=O)c1cc(CN)c(-n2ccnc2)nn1] | ++ | MS-ESI: m/z 389.49 observed [M + H]+ |
| 66 | 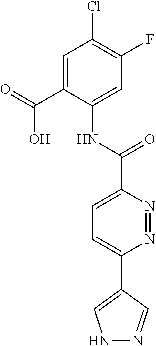 [SMILES: O=C(Nc1cc(F)c(Cl)cc1C(=O)O)c1ccc(-c2cn[nH]c2)nn1] | ++ | MS-ESI: m/z 362.44 observed [M + H]+ |
| 67 | 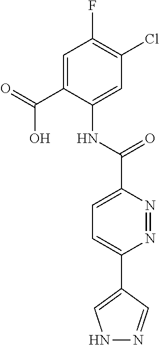 [SMILES: O=C(Nc1cc(Cl)c(F)cc1C(=O)O)c1ccc(-c2cn[nH]c2)nn1] | ++ | MS-ESI: m/z 362.44 observed [M + H]+ |
| 68 | 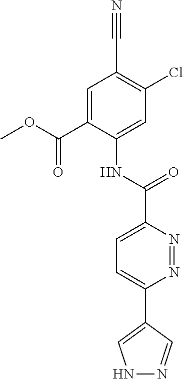 [SMILES: COC(=O)c1cc(C#N)c(Cl)cc1NC(=O)c1ccc(-c2cn[nH]c2)nn1] | ++++ | MS-ESI: m/z 383.46 observed [M + H]+ |
| 69 | 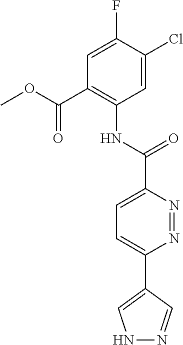 [SMILES: COC(=O)c1cc(F)c(Cl)cc1NC(=O)c1ccc(-c2cn[nH]c2)nn1] | ++++ | MS-ESI: m/z 376.45 observed [M + H]+ |
| 70 | 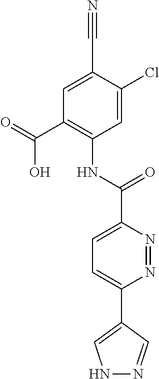 [SMILES: N#Cc1cc(C(=O)O)c(NC(=O)c2ccc(-c3cn[nH]c3)nn2)cc1Cl] | + | MS-ESI: m/z 369.47 observed [M + H]+ |
| 71 | 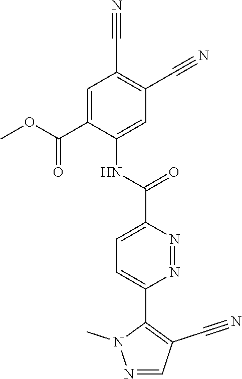 [SMILES: COC(=O)c1cc(C#N)c(C#N)cc1NC(=O)c1ccc(-c2c(C#N)cnn2C)nn1] | + | MS-ESI: m/z 413.49 observed [M + H]+ |
| 72 | 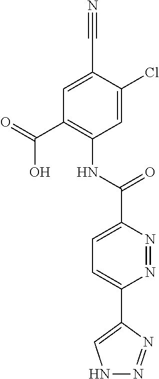 [SMILES: N#Cc1cc(C(=O)O)c(NC(=O)c2ccc(-c3c[nH]nn3)nn2)cc1Cl] | + | MS-ESI: m/z 370.49 observed [M + H]+ |
| 73 | 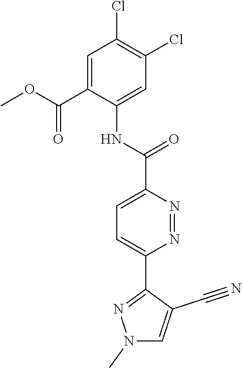 [SMILES: COC(=O)c1cc(Cl)c(Cl)cc1NC(=O)c1ccc(-c2nn(C)cc2C#N)nn1] | + | ¹H NMR (400 MHz, DMSO-d₆) δ 12.99 (s, 1H), 9.07 (s, 1H), 8.80 (s, 1H), 8.47 (d, J = 8.8 Hz, 1H), 8.41 (d, J = 8.8 Hz, 1H), 8.21 (s, 1H), 4.05 (s, 3H), 3.98 (s, 3H). MS-ESI: m/z 431.10 observed [M + H]+ |
| 74 | 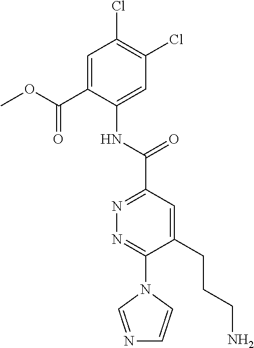 [SMILES: COC(=O)c1cc(Cl)c(Cl)cc1NC(=O)c1cc(CCCN)c(-n2ccnc2)nn1] | ++ | MS-ESI: m/z 449.48 observed [M + H]+ |
| 75 | 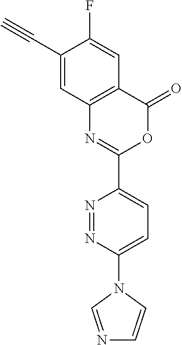 [SMILES: C#Cc1cc2nc(-c3ccc(-n4ccnc4)nn3)oc(=O)c2cc1F] | ++++ | ¹H NMR (400 MHz, DMSO-d₆) δ 8.86-8.74 (m, 1H), 8.64 (d, J = 9.3 Hz, 1H), 8.45 (d, J = 9.5 Hz, 1H), 8.28-8.14 (m, 1H), 8.11 (d, J = 8.5 Hz, 1H), 8.04 (d, J = 6.3 Hz, 1H), 7.33-7.16 (m, 1H), 5.02 (s, 1H). MS-ESI: m/z 334.76 observed [M + H]+ |
| 76 | 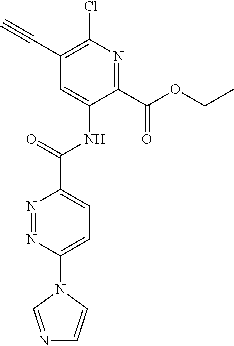 [SMILES: C#Cc1cc(NC(=O)c2ccc(-n3ccnc3)nn2)c(C(=O)OCC)nc1Cl] | ++++ | ¹H NMR (400 MHz, DMSO-d₆) δ 12.85 (s, 1H), 9.31 (s, 1H), 8.84 (s, 1H), 8.56 (d, J = 9.2 Hz, 1H), 8.49 (d, J = 8.8 Hz, 1H), 8.24 (s, 1H), 7.28 (s, 1H), 5.14 (s, 1H), 4.46 (q, J = 6.8 Hz, 2H), 1.39 (t, J = 6.8 Hz, 3H) MS-ESI: m/z 397.4 observed [M + H]+ |
| 77 | 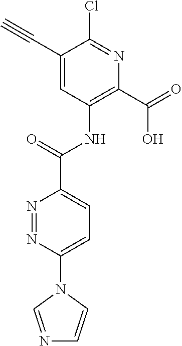 [SMILES: C#Cc1cc(NC(=O)c2ccc(-n3ccnc3)nn2)c(C(=O)O)nc1Cl] | ++++ | ¹H NMR (400 MHz, DMSO-d₆) δ 15.47 (s, 1H), 9.25 (d, J = 1.5 Hz, 1H), 8.80 (q, J = 1.2 Hz, 1H), 8.48 (dd, J = 9.1, 1.4 Hz, 1H), 8.42 (dd, J = 9.2, 1.4 Hz, 1H), 8.21 (q, J = 1.4 Hz, 1H), 7.26 (q, J = 1.2 Hz, 1H), 4.81 (d, J = 1.6 Hz, 1H). MS-ESI: m/z 369.44 observed [M + H]+ |
| 78 | 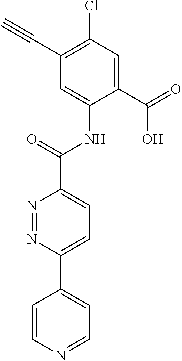 [SMILES: C#Cc1cc(NC(=O)c2ccc(-c3ccncc3)nn2)c(C(=O)O)cc1Cl] | ++++ | ¹H NMR (400 MHz, DMSO-d₆) δ 15.88 (s, 1H), 8.99 (s, 1H), 8.84 (d, J = 6 Hz, 2H), 8.60 (d, J = 9.2 Hz, 1H), 8.42 (d, J = 8.8 Hz, 1H), 8.24 (d, J = 6 Hz, 2H), 8.08 (s, 1H), 4.60 (s, 1H) MS-ESI: m/z 379.1 observed [M + H]+ |
| 79 | 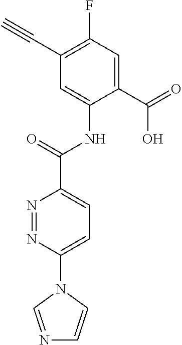 [SMILES: C#Cc1cc(NC(=O)c2ccc(-n3ccnc3)nn2)c(C(=O)O)cc1F] | ++++ | ¹H NMR (400 MHz, DMSO-d₆) δ 13.11 (s, 1H), 9.44 (s, 1H), 8.99 (d, J = 6.6 Hz, 1H), 8.64 (d, J = 9.0 Hz, 1H), 8.55 (d, J = 9.2 Hz, 1H), 8.44 (s, 1H), 7.90 (d, J = 9.8 Hz, 1H), 7.60 (s, 1H), 4.85 (s, 1H). MS-ESI: m/z 352.3 observed [M + H]+ |
| 80 | 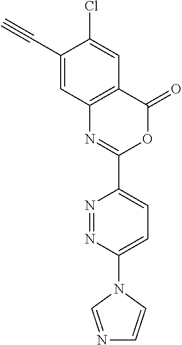 [SMILES: C#Cc1cc2nc(-c3ccc(-n4ccnc4)nn3)oc(=O)c2cc1Cl] | ++++ | ¹H NMR (400 MHz, MeOD) δ 9.93 (s, 1H), 8.90 (d, J = 9.0 Hz, 1H), 8.57 (s, 1H), 8.50 (d, J = 9.2 Hz, 1H), 8.35 (d, J = 1.7 Hz, 1H), 8.04 (s, 1H), 7.85 (s, 1H), 4.40 (s, 1H). MS-ESI: m/z 350.59 observed [M + H]+ |
| 81 | 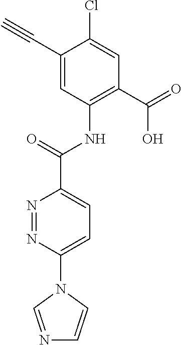 [SMILES: C#Cc1cc(NC(=O)c2ccc(-n3ccnc3)nn2)c(C(=O)O)cc1Cl] | ++++ | ¹H NMR (400 MHz, DMSO-d₆) δ 15.87 (s, 1H), 8.97 (s, 1H), 8.80 (s, 1H), 8.46 (d, J = 9.1 Hz, 1H), 8.41 (d, J = 9.1 Hz, 1H), 8.21 (s, 1H), 8.06 (s, 1H), 7.26 (s, 1H), 4.61 (s, 1H). MS-ESI: m/z 368.48 observed [M + H]+ |
| 82 | 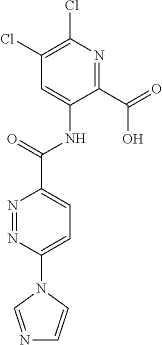 [SMILES: O=C(Nc1cc(Cl)c(Cl)nc1C(=O)O)c1ccc(-n2ccnc2)nn1] | ++++ | ¹H NMR (400 MHz, DMSO-d₆) δ 15.74 (s, 1H), 9.30 (t, J = 1.0 Hz, 1H), 8.81 (d, J = 1.5 Hz, 1H), 8.61-8.36 (m, 2H), 8.22 (d, J = 1.6 Hz, 1H), 7.27 (d, J = 2.3 Hz, 1H), MS-ESI: m/z 380.16 observed [M + H]+ |
| 83 | 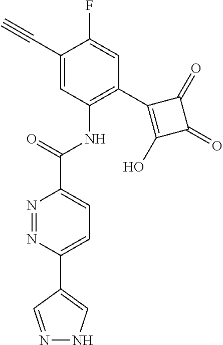 [SMILES: C#Cc1cc(NC(=O)c2ccc(-c3cn[nH]c3)nn2)c(-c2c(O)c(=O)c2=O)cc1F] | ++++ | ¹H NMR (400 MHz, DMSO-d₆) δ 15.01 (s, 1H), 8.50 (s, 2H), 8.47 (d, J = 6.8, 1H), 8.20- 8.14 (m, 2H), 8.01 (d, J = 8.1, 1H), 4.63 (s, 1H). MS-ESI: m/z 404.2 observed [M + H]+ |
| 84 | 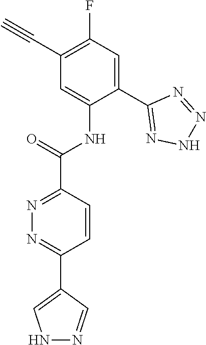 [SMILES: C#Cc1cc(NC(=O)c2ccc(-c3cn[nH]c3)nn2)c(-c2nn[nH]n2)cc1F] | ++ | ¹H NMR(499 MHz, DMSO-d₆) δ 12.79 (s, 1H), 8.96 (d, J = 6.8 Hz, 1H), 8.32 (s, 1H), 8.32- 8.24 (m, 2H), 8.03 (d, J = 10 Hz, 1H), 4.78 (s, 1H) MS-ESI: m/z 376.1 observed [M + H]+ |
| 85 | 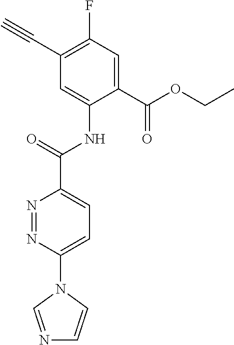 [SMILES: C#Cc1cc(NC(=O)c2ccc(-n3ccnc3)nn2)c(C(=O)OCC)cc1F] | ++++ | ¹H NMR (499 MHz, DMSO-d₆) δ 12.84 (s, 1H), 8.94 (d, J = 6.4 Hz, 2H), 8.82 (s, 1H), 8.55- 8.45 (m, 2H), 8.22 (s, 1H), 7.90 (d, J = 10 Hz, 1H), 7.27 (s, 1H), 4.88 (s, 1H), 4.42 (q, J = 6.8 Hz, 1H), 1.39 (t, J = 6.8 Hz, 3H) MS-ESI: m/z 380.0 observed [M + H]+ |
| 86 | 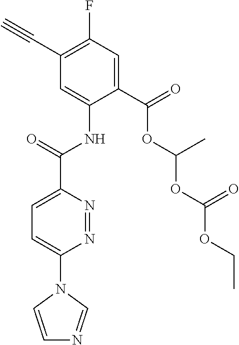 [SMILES: C#Cc1cc(NC(=O)c2ccc(-n3ccnc3)nn2)c(C(=O)OC(C)OC(=O)OCC)cc1F] | ++++ | ¹H NMR (499 MHz, DMSO-d₆) δ 12.65 (s, 1H), 8.93 (d, J = 6 Hz, 2H), 8.58 (d, J = 8.8 Hz, 1H), 8.50 (d, J = 9.2 Hz, 1H), 8.27 (s, 1H), 7.94 (d, 9.6 Hz, 1H), 7.32 (s, 1H), 6.98-6.94 (m, 1H), 4.93 (s, 1H), 4.18 (q, J = 7.2 Hz, 2H), 1.64 (d, J = 5.2 Hz, 3H), 1.22 (t, J = 7.2 Hz, 3H) MS-ESI: m/z 468.3 observed [M + H]+ |
| 87 | 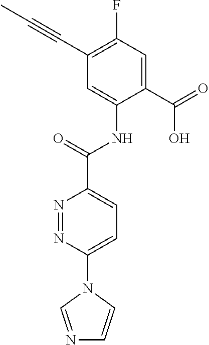 [SMILES: CC#Cc1cc(NC(=O)c2ccc(-n3ccnc3)nn2)c(C(=O)O)cc1F] | ++++ | ¹H NMR (400 MHz, DMSO-d₆) δ 15.81 (s, 1H), 8.97-8.66 (m, 2H), 8.53-8.28 (m, 2H), 8.21 (d, J = 1.5 Hz, 1H), 7.74 (d, J = 10.4 Hz, 1H), 7.27 (d, J = 2.2 Hz, 1H), 2.13 (s, 3H). MS-ESI: m/z 366.5 observed [M + H]+ |
| 88 | 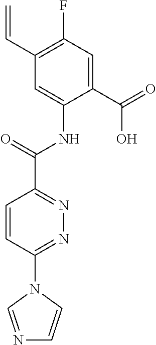 [SMILES: C=Cc1cc(NC(=O)c2ccc(-n3ccnc3)nn2)c(C(=O)O)cc1F] | ++++ | ¹H NMR (400 MHz, DMSO-d₆) δ 15.84 (s, 1H), 9.05-8.97 (m, 1H), 8.80 (d, J = 1.4 Hz, 1H), 8.47 (dd, J = 9.0, 1.4 Hz, 1H), 8.41 (dd, J = 9.1, 1.4 Hz, 1H), 8.21 (d, J = 1.6 Hz, 1H), 7.71 (dd, J = 11.7, 1.4 Hz, 1H), 7.26 (q, 1.2 Hz, 1H), 6.97-6.77 (m, 1H), 5.87 (d, J = 17.6 Hz, 1H), 5.48 (d, J = 11.0 Hz, 1H). MS-ESI: m/z 354.49 observed [M + H]+ |
| 89 | 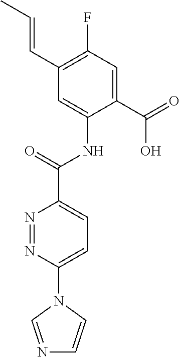 [SMILES: C/C=C/c1cc(NC(=O)c2ccc(-n3ccnc3)nn2)c(C(=O)O)cc1F] | ++++ | ¹H NMR (400 MHz, DMSO-d₆) δ 15.79 (d, J = 2.2 Hz, 1H), 8.94 (dd, J = 7.4, 1.9 Hz, 1H), 8.80 (q, J = 1.3 Hz, 1H), 8.43 (qt, J = 9.1, 1.4 Hz, 2H), 8.21 (q, J = 1.5 Hz, 1H), 7.68 (dd, J = 11.7, 2.0 Hz, 1H), 7.26 (d, J = 1.6 Hz, 1H), 6.54 (dd, J = 15.9, 2.2 Hz, 1H), 6.40-6.30 (m, 1H). MS-ESI: m/z 368.49 observed [M + H]+ |
| 90 | 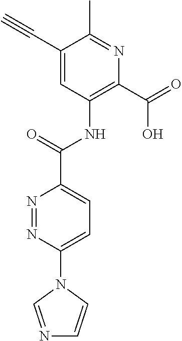 [SMILES: C#Cc1cc(NC(=O)c2ccc(-n3ccnc3)nn2)c(C(=O)O)nc1C] | +++ | ¹H NMR (400 MHz, DMSO-d₆) δ 15.40 (s, 1H), 9.09 (s, 1H), 8.80 (s, 1H), 8.48-8.41 (m, 2H), 8.21 (s, 1H), 7.27 (s, 1H), 4.62 (s, 1H), 2.57 (s, 3H). MS-ESI: m/z 349.0 observed [M + H]+ |
| 91 | 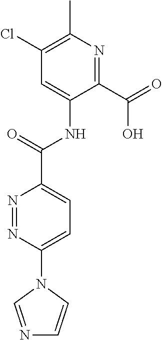 [SMILES: Cc1nc(C(=O)O)c(NC(=O)c2ccc(-n3ccnc3)nn2)cc1Cl] | ++ | ¹H NMR (400 MHz, DMSO-d₆) δ 15.35 (s, 1H), 9.19 (s, 1H), 8.80 (s, 1H), 8.49-8.41 (m, 2H), 8.21 (s, 1H), 7.26 (s, 1H). MS-ESI: m/z 359.1 observed [M + H]+ |
| 92 | 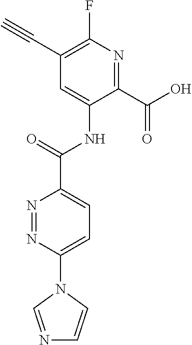 [SMILES: C#Cc1cc(NC(=O)c2ccc(-n3ccnc3)nn2)c(C(=O)O)nc1F] | ++++ | ¹H NMR (400 MHz, DMSO-d₆) δ 15.32 (s, 1H), 9.31 (d, J = 8 Hz, 1H), 8.80 (s, 1H), 8.50- 8.43 (m, 1H), 8.41- 8.37 (m, 1H), 8.21 (s, 1H), 7.27 (s, 1H), 4.714 (s, 1H) MS-ESI: m/z 353.2 observed [M + H]+ |
| 93 | 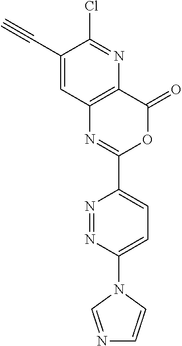 [SMILES: C#Cc1cc2nc(-c3ccc(-n4ccnc4)nn3)oc(=O)c2nc1Cl] | +++ | ¹H NMR (400 MHz, DMSO-d₆) δ 8.81 (s, 1H), 8.65 (d, J = 9.3 Hz, 1H), 8.55 (s, 1H), 8.47 (d, J = 9.3 Hz, 1H), 8.21 (t, J = 1.5 Hz, 1H), 7.33-7.23 (m, 1H), 5.26 (s, 1H). MS-ESI: m/z 351.1 observed [M + H]+ |
| 94 | 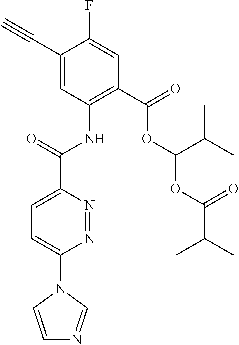 [SMILES: C#Cc1cc(NC(=O)c2ccc(-n3ccnc3)nn2)c(C(=O)OC(OC(=O)C(C)C)C(C)C)cc1F] | ++++ | ¹H NMR (400 MHz, DMSO-d₆) δ 12.65 (s, 1H), 8.94 (d, J = 6.4 Hz, 1H), 8.83 (s, 1H), 8.56 (d, J = 9.2 Hz, 1H), 8.48 (d, J = 9.2 Hz, 1H), 8.24 (s, 1H), 7.87 (d, J = 9.6 Hz, 1H), 7.28 (s, 1H), 6.85 (d, J = 4.8 Hz, 1H), 4.93 (s, 1H), 2.68-2.61 (m, 1H), 2.24-2.19 (m, 1H), 1.14-1.09 (m, 6H), 1.03-0.87 (m, 6H). MS-ESI: m/z 494.3 observed [M + H]+ |
| 95 | 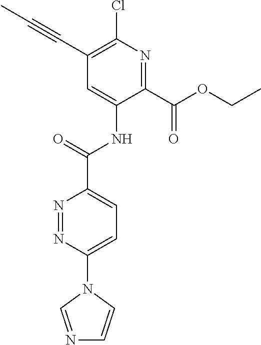 [SMILES: CC#Cc1cc(NC(=O)c2ccc(-n3ccnc3)nn2)c(C(=O)OCC)nc1Cl] | ++++ | ¹H NMR (400 MHz, DMSO-d₆) δ 12.87 (s, 1H), 9.25 (s, 1H), 8.86 (s, 1H), 8.57 (d, J = 9.2 Hz, 1H), 8.50 (d, J = 9.2 Hz, 1H), 8.26 (s, 1H), 7.29 (s, 1H), 4.46 (q, J = 6.8 Hz, 2H), 2.25 (s, 3H), 1.40 (t, J = 6.8 Hz, 3H). MS-ESI: m/z 411.6 observed [M + H]+ |
| 96 | 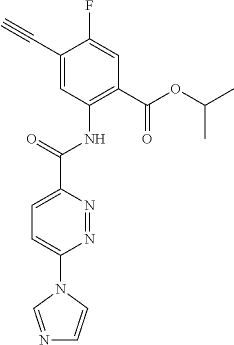 [SMILES: C#Cc1cc(NC(=O)c2ccc(-n3ccnc3)nn2)c(C(=O)OC(C)C)cc1F] | ++ | ¹H NMR (400 MHz, DMSO-d₆) δ 12.84 (s, 1H), 8.94 (d, J = 6.5 Hz, 1H), 8.87-8.79 (m, 1H), 8.55 (d, J = 9.1 Hz, 1H), 8.47 (d, J = 9.1 Hz, 1H), 8.28-8.19 (m, 1H), 7.90 (d, J = 9.8 Hz, 1H), 7.27 (s, 1H), 5.26 (p, J = 6.2 Hz, 1H), 4.87 (s, 1H), 1.38 (d, J = 6.2 Hz, 5H). MS-ESI: m/z 394.1 observed [M + H]⁺ |
| 97 | 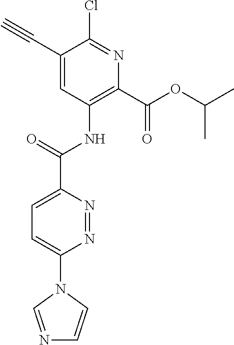 [SMILES: C#Cc1cc(NC(=O)c2ccc(-n3ccnc3)nn2)c(C(=O)OC(C)C)nc1Cl] | ++++ | ¹H NMR (400 MHz, DMSO-d₆) δ 12.83 (s, 1H), 9.29 (s, 1H), 8.84 (s, 1H), 9.56 (d, J = 9.2 Hz, 1H), 8.49 (d, J = 8.8 Hz, 1H), 8.24 (s, 1H), 7.28 (s, 1H), 5.32- 5.259 (m, 1H), 5.12 (s, 1H), 1.39 (d, J = 6 Hz, 6H) MS-ESI: m/z 411.5 observed [M + H]⁺ |
| 98 | 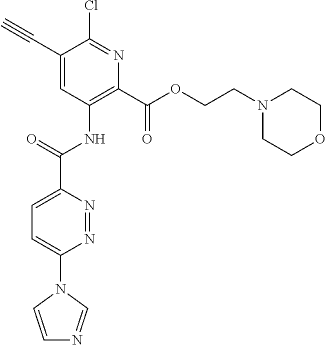 [SMILES: C#Cc1cc(NC(=O)c2ccc(-n3ccnc3)nn2)c(C(=O)OCCN2CCOCC2)nc1Cl] | ++++ | ¹H NMR(400 MHz, DMSO-d₆) δ 13.18 (s, 1H), 9.57 (s, 1H), 8.63- 8.56 (m, 2H), 7.89-7.82 (m, 2H), 7.38 (s, 1H), 4.73 (s, 2H), 3.78-3.73 (m, 4H), 2.97 (m, 2H), 2.68 (s, 3H) MS-ESI: m/z 482.3 observed [M + H]⁺ |
| 99 | 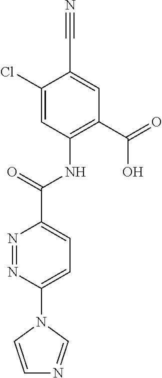 [SMILES: N#Cc1cc(C(=O)O)c(NC(=O)c2ccc(-n3ccnc3)nn2)cc1Cl] | +++ | ¹H NMR (400 MHz, DMSO-d₆) δ 16.49 (s, 1H), 9.01 (d, J = 1.8 Hz, 1H), 8.81 (s, 1H), 8.52- 8.35 (m, 3H), 8.22 (q, J = 1.4 Hz, 1H), 7.26 (s, 1H). MS-ESI: m/z 369.43 observed [M + H]⁺ |
| 100 | 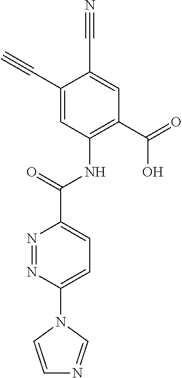 [SMILES: C#Cc1cc(NC(=O)c2ccc(-n3ccnc3)nn2)c(C(=O)O)cc1C#N] | +++ | ¹H NMR (400 MHz, DMSO-d₆) δ 16.32 (s, 1H), 9.02 (s, 1H), 8.80 (s, 1H), 8.49-8.47 (m, 1H), 8.43-8.41 (m, 1H), 8.35 (s, 1H), 8.21 (s, 1H), 7.26 (s, 1H), 4.79 (s, 1H) MS-ESI: m/z 359.47 observed [M + H]⁺ |
| 101 | 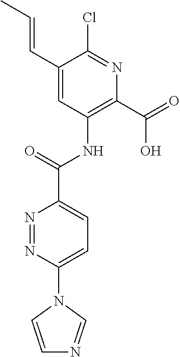 [SMILES: C/C=C/c1cc(NC(=O)c2ccc(-n3ccnc3)nn2)c(C(=O)O)nc1Cl] | ++++ | MS-ESI: m/z 385.35 observed [M + H]⁺ |
| 102 | 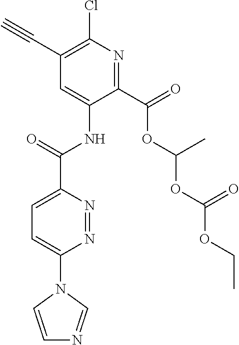 [SMILES: C#Cc1cc(NC(=O)c2ccc(-n3ccnc3)nn2)c(C(=O)OC(C)OC(=O)OCC)nc1Cl] | ++++ | ¹H NMR (400 MHz, DMSO-d₆) δ 12.66 (s, 1H), 9.30(s, 1H), 8.85 (s, 1H), 8.58 (d, J = 9.2 Hz, 1H), 8.50 (d, J = 8.8 Hz, 1H), 8.25 (s, 1H), 7.28 (s, 1H), 7.03- 6.99 (m, 1H), 5.18 (s, 1H), 4.19 (q, J = 7.2, 2H), 1.64 (d, J = 5.6 Hz, 3H), 1.25-1.14 (m, 3H) MS-ESI: m/z 485.6 observed [M + H]⁺ |
| 103 | 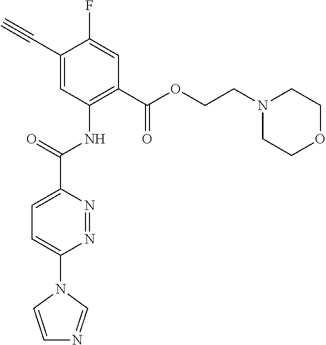 [SMILES: C#Cc1cc(NC(=O)c2ccc(-n3ccnc3)nn2)c(C(=O)OCCN2CCOCC2)cc1F] | ++++ | ¹H NMR (400 MHz, DMSO-d₆) δ 12.79 (s, 1H), 9.04-8.95 (m, 1H), 8.93 (s, 1H), 8.69-8.56 (m, 1H), 8.54-8.49 (m, 1H), 8.23 (s, 1H), 7.89 (d, J = 9.6 Hz, 1H), 7.28 (s, 1H), 4.89 (s, 1H), 4.49 (t, J = 5.2 Hz, 2H), 3.57-3.52 (m, 6H), 2.76 (t, J = 5.2 Hz, 4H) MS-ESI: m/z 465.2 observed [M + H]⁺ |
| 104 | 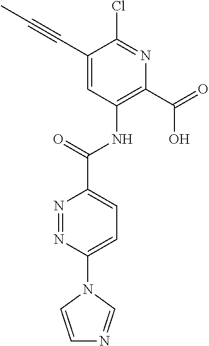 [SMILES: CC#Cc1cc(NC(=O)c2ccc(-n3ccnc3)nn2)c(C(=O)O)nc1Cl] | ++++ | ¹H NMR (400 MHz, DMSO-d₆) δ 15.54 (s, 1H), 9.19 (s, 1H), 8.82 (s, 1H), 8.47-8.42 (m, 2H), 8.23 (s, 1H), 7.28 (s, 1H), 2.19 (s, 3H) MS-ESI: m/z 383.4 observed [M + H]⁺ |
| 105 | 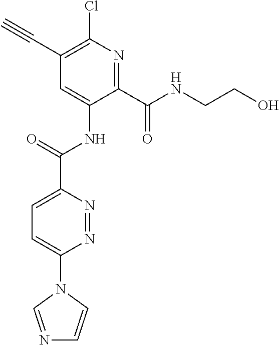 [SMILES: C#Cc1cc(NC(=O)c2ccc(-n3ccnc3)nn2)c(C(=O)NCCO)nc1Cl] | ++ | ¹H NMR(400 MHz, DMSO-d₆) δ 14.06 (s, 1H), 9.44 (s, 1H), 9.01 (s, 1H), 8.86 (s, 1H), 8.52 (dd, J = 9.2, 30 Hz, 2H), 8.26 (s, 1H), 7.29 (s, 1H), 5.09 (s, 1H), 4.89 (t, J = 5.2 Hz, 1H), 3.58 (d, J = 5.6 Hz, 2H), 3.45 (d, J = 5.6 Hz) MS-ESI: m/z 412.3 observed [M + H]⁺ |
| 106 | 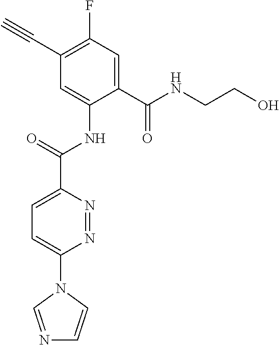 [SMILES: C#Cc1cc(NC(=O)c2ccc(-n3ccnc3)nn2)c(C(=O)NCCO)cc1F] | + | ¹H NMR (400 MHz, DMSO-d₆) δ 13.23 (s, 1H), 8.95-8.94 (m, 1H), 8.9-8.89 (m, 1H), 8.83 (s, 1H), 8.49 (dd, J = 9.2, 27.6 Hz, 2H), 8.23 (s, 1H), 7.86 (d, J = 10 Hz, 1H), 7.27 (s, 1H), 4.83 (t, J =6 Hz, 1H), 4.78 (s, 1H), 3.56 (dd, J = 6, 11.6 Hz, 2H), 3.38 (dd,J= 5.6, 11.6 Hz, 2H) MS-ESI: m/z 395.3 observed [M + H]⁺ |
| 107 | 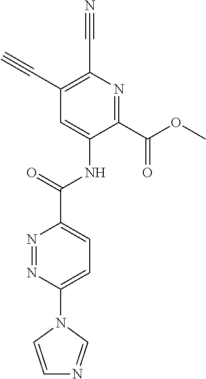 [SMILES: C#Cc1cc(NC(=O)c2ccc(-n3ccnc3)nn2)c(C(=O)OC)nc1C#N] | ++++ | ¹H NMR (400 MHz, DMSO-d₆) δ 13.07 (s, 1H), 9.29 (s, 1H), 8.81 (s, 1H), 8.50 (d, J = 8.8 Hz, 1H), 8.44 (d, J = 8.0 Hz, 1H), 8.21 (s, 1H), 7.26 (s, 1H), 5.24 (s, 1H), 3.98 (s, 3H). MS-ESI: m/z 373.9 observed [M + H]⁺ |
| 108 | 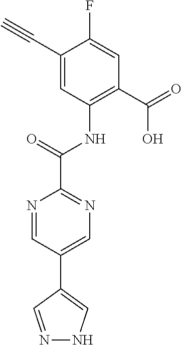 [SMILES: C#Cc1cc(NC(=O)c2ncc(-c3cn[nH]c3)cn2)c(C(=O)O)cc1F] | + | ¹H NMR (400 MHz, DMSO-d₆) δ 9.90 (s, 2H), 9.01 (s, 2H), 8.99- 8.98 (m, 1H), 8.14-8.09 (m, 1H), 3.67 (d, J = 24.8 Hz, 1H) MS-ESI: m/z 352.2 observed [M + H]⁺ |

### RELATED DOCUMENTS

[1] Corrales L, Glickman L H, McWhirter S M, Kanne D B, Sivick K E, Katibah G E, Woo S R, Lemmens E, Banda T, Leong J J, Metchette K, Dubensky T W Jr, Gajewski T F. (2015) Direct Activation of STING in the Tumor Microenvironment Leads to Potent and Systemic Tumor Regression and Immunity. Cell Rep. 11: 1018-30.
[2] Deng, L. et al. (2014) STING-Dependent Cytosolic DNA Sensing Promotes Radiation-Induced Type I Interferon-Dependent Antitumor Immunity in Immunogenic Tumors, Immunity. 41: 843.
[3] Corrales L, Matson V, Flood B, Spranger S, Gajewski T F. (2017) Innate immune signaling and regulation in cancer immunotherapy. Cell Res. 27: 96-108.
[4] Corrales L, McWhirter S M, Dubensky T W Jr, Gajewski T F. (2016) The host STING pathway at the interface of cancer and immunity. J Clin Invest. 126: 2404-11.

Methods of Use

The present disclosure also provides in an embodiment a method of stimulating expression of interferon genes in a human patient. The method comprises administering to the patient an effective dose of a compound or pharmaceutically acceptable salt thereof as described herein.

In another embodiment, the present disclosure provides a method of treating a tumor in a patient. The method comprises administering to the patient an effective dose of a compound or pharmaceutically acceptable salt thereof.

With respect to combination therapies comprising administration of a compound of the present disclosure and an immune-checkpoint targeting drug, or as combination therapies for the potentiation of ionizing radiation-based and existing chemotherapies therapeutic approaches, such as DNA-damage-based chemotherapies, the STING agonists of the present disclosure can complement and potentiate the effects of these known therapeutic approaches. This is based on recent papers indicating the critical role of STING-dependent micronuclei-mediated tumor clearance using these approaches, see for example:
[5] Mackenzie, K. F., et all, (2017), cGAS surveillance of micronuclei links genome instability to innate immunity, Nature, 548, 461.
[6] Wang, W. et al., (2016), Effector T Cells Abrogate Stroma-Mediated Chemoresistance in Ovarian Cancer, Cell, 165, 1092-1105.
[7] Charlotte E. Ariyan, et al., Jan. 16, 2018; DOI: 10.1158/2326-6066, Robust antitumor responses result from local chemotherapy and CTLA-4 blockade, cancerimmunolres.aacrjournals.org on Jan. 31, 2018.
[8] Chung Kil Song, et al., www.moleculartherapy.org vol. 15 no. 8 Aug. 2007, Chemotherapy Enhances CD8+ T Cell-mediated Antitumor Immunity Induced by Vaccination With Vaccinia Virus.

Compounds of the present disclosure can be used in therapeutic combinations with administration of an effective dose of an immune-checkpoint targeting drug. For example, the immune-checkpoint targeting drug can be an anti-PD-L1 antibody, anti-PD-1 antibody, anti-CTLA-4 antibody, or an anti-4-1BB antibody. See, for example:
[9] Ager, C R, et al., (2017) Cancer Immunol Res; 5(8), 676.
[10] Fu, J. et al. (2015) Sci Transl Med. 2015 Apr. 15; 7(283): 283ra52. doi:10.1126/scitranslmed.aaa4306.
[11] Wang, H., et al. (2017) PNAS, Feb. 14, 2017, vol. 114, no. 7, 1637-1642.

Pharmaceutical Composition

The present disclosure provides in another embodiment a pharmaceutical composition comprising a compound or pharmaceutically acceptable salt thereof as described herein in combination with a pharmaceutically acceptable carrier or excipient.

Compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of the compound or a pharmaceutically acceptable salt thereof.

For tablet compositions, the compound or a pharmaceutically acceptable salt thereof in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, the compound or a pharmaceutically acceptable salt thereof is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compound or a pharmaceutically acceptable salt thereof in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the compound or a pharmaceutically acceptable salt thereof in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may he formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may he formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compound the compound or a pharmaceutically acceptable salt thereof may also he administered in the form of suppositories for rectal administration. These compositions can be prepared by mixing the compound with a suitable non-irritating excipient which is solid at ordinary temperatures but liquid at the rectal temperature and will therefore melt in the rectum to release the compound. Exemplary excipients include cocoa butter and polyethylene glycols.

Compositions for parenteral administrations are administered in a sterile medium. Depending on the vehicle used and concentration the concentration of the compound or a pharmaceutically acceptable salt thereof in the formulation, the parenteral formulation can either be a suspension or a solution containing dissolved compound. Adjuvants such as local anesthetics, preservatives and buffering agents can also be added to parenteral compositions.

### EXAMPLES

The following non-limiting examples are additional embodiments for illustrating the present disclosure.

Tissue culture. Wild-type (cat. no. thpl-isg) and STING KO (cat. no. thpd-kostg) THP-1-Lucia ISG cells were purchased from Invivogen and maintained in growth media consisting of RPMI 1640, 2 mM L-glutamine, 25 mM HEPES, 10% heat-inactivated fetal bovine serum (FBS), 1,000 units/ml penicillin, 1,000 μg/ml streptomycin, 0.25 μg/ml Amphotericin B, and 100 μg/ml zeocin unless otherwise stated.

Type 1 interferon stimuli. Poly(dA:dT) and 2′,3′-cGAMP were purchased from invivogen and resuspended according to manufacturer's instructions.

ISRE-luciferase assay. THP-1 Lucia ISG cells were resuspended in low-serum growth media (2% FBS) at a density of 5×10⁵ cells/ml and treated with test article or vehicle (DMSO). 50 μL of cells were seeded into each well of a 384-well white greiner plates and incubated for 24 hours. To evaluate expression of the luciferase reporter, 30 μl of Quanti-luc (Invivogen) detection reagent was added to each well and luminescence was read using an Envision plate reader (Perkin Elmer) set with an integration time of 0.1 seconds.

Viability assay. Cells were resuspended in low-serum growth media at a density of 5×10⁵ cells/ml and treated with test article or vehicle (DMSO). 50 μL of cells were seeded into each well of a 384-well white greiner plates and incubated for 24 hours. To evaluate expression of the luciferase reporter, 30 μl of CellTiter-Glo (Promega) detection reagent was added to each well and luminescence was detected \using an Envision Plate Reader set with an integration time of 0.1 seconds.

Western Blot. Cells were solubilized in 1× protein lysis buffer (25 mM HEPES, pH 7.4, 300 mM, NaCl, 1.5 mM MgCl₂, 1 mM EGTA, 1% P-40, 1% sodium deoxycholate, 2.5 mM sodium pyrophosphate, 1 mM glycerophosphate) with freshly added protease and phosphatase inhibitors (Cell Signaling). Western blotting was performed using Bolt™ 4-12% Bis-Tris gels and Bolt™ mini transfer system following the manufacturer's instructions (ThermoFisher Scientific). STING and γ-tubulin antibodies were purchased from Cell Signaling diluted in 5% BSA, 1× TBS-T buffer (Table 3). Anti-rabbit HRP antibody was diluted in 5% non-fat dried milk, 1× TBS-T buffer and luminescence signal was imaged using a ChemiDoc Imager (BioRad).

Semi-quantitative real-time PCR (qPCR). THP-1 cells were resuspended in low-serum growth media at a density of 5×10⁵ cells/ml and treated with test article or vehicle (DMSO). 2.5 mL of cells were seeded into each well of a 6-well plate and incubated for 24 hours. RNA was isolated using an RNeasy Plus Mini Kit (Qiagen) and 1 μg of purified RNA was reverse-transcribed into cDNA (VILO, cat. no. 11755050, ThermoFisher Scientific). Gene expression was assessed using Taqman primers and probes listed in Table 4 with the Taqman Universal Mix II (cat. no. 4440038, ThermoFisher) following manufacturer's instructions. Gene expression was normalized using the double delta Ct method and was reported as fold change in expression.

STING Thermal Shift Assay (TSA). The c-terminal domains (CTD) of human and mouse STING were expressed and purified as detailed previously (Ouyang, S., Song, X., Wang, Y., Ru, H., Shaw, N., Jiang, Y., Niu, F., Zhu, Y., Qiu, W., Parvatiyar, K., et al. (2012). Structural analysis of the STING adaptor protein reveals a hydrophobic dimer interface and mode of cyclic di-GMP binding. Immunity 36, 1073-1086.). Test article or vehicle controls were added to diluted STING protein (0.22 mg/ml) in 1× Protein Thermal Shift Buffer provided in the Protein Thermal Shift Dye Kit (cat #4461146, ThermoFisher Scientific). Thermal Shift dye was added and mixed prior to performing a melt curve following parameters outlined for the Dye kit. Melt temperatures (Tm) were calculated using the Derivative method using Protein Thermal Shift Software v1.3 (cat #4466038, ThermoFisher Scientific).

WT STING binding assay (Cisbio, Catalog #64BDSTGPEH). An assay format was optimized to demonstrate binding of recombinant 6× His-tagged human STING protein labeled with Terbium Cryptate by the natural ligand, 2′3′cGAMP labeled with d2 (the acceptor). Upon proximity of the two dyes, the excitation of the donor by the flash lamp on the PHERAstar FSX plate reader triggers a Fluorescence Resonance Energy Transfer (FRET) towards the acceptor, which in turn fluoresces at 665 nm. To assess the ability of the synthetic small molecule STING ligands to bind to human STING, a competitive assay format was applied. A 10-point titration of each of the synthetic ligands in 5 uL were transferred into a 384 well plate, followed by 20 uL of assay buffer containing the 6× His-tagged human STING protein and labeled 2′3′cGAMP ligand and incubated for three hours at room temperature. The raw values obtained from the PHERAstar were used to calculate the reported IC₅₀ values (the signal is inversely proportional to the binding of the synthetic ligand) through curve fitting in Genedata. The percent inhibition was calculated based upon the maximal amount of binding by synthetic compound versus the maximum binding of unlabeled 2′3′ cGAMP which was used as a control in each assay.

TABLE 2
Cell Signaling Antibodies

| Protein target | Cat. No. | Dilution |
|---|---|---|
| STING | 13647 | 1:1000 |
| γ-tubulin | 5886 | 1:3000 |
| Rabbit IgG | 7074 | 1:3000 |

TABLE 3
ThermoFisher Scientific Taqman Primers/Probe

| Gene Symbol | Species | Cat. No. | Dye |
|---|---|---|---|
| IFNB1 | human | Hs01077958_s1 | FAM |
| CXCL10 | human | Hs00171042_m1 | FAM |
| IFIT3 | human | Hs01922752_s1 | FAM |
| B2M | human | Hs00187842_m1 | VIC |

Compounds useful for carrying out a method of the present disclosure can be prepared according to the following procedures in conjunction with ordinary knowledge and skill in organic synthesis, substituting appropriate reagents as apparent to the practitioner.

Experimental Procedures

Abbreviations. The following abbreviations are used: tetrahydrofuran (THF), dichloromethane (DCM), NN-dimethylformamide (DMF), dimethylacetamide (DMA), dimethylsulfoxide (DMSO), trifluoroacetic acid (TFA), triethylamine (TEA), diisopropylethylamine (DIPEA), (1-Cyano-2-ethoxy-2-oxoethylidenaminooxy)dimethylamino-morpholino-carbenium hexafluorophosphate (COMU), 1-[bis(dimethylamino)methylene]-1H-1,2,3-triazolo[4,5-b]pyridinium 3-oxid hexafluorophosphate, N-[(dimethylamino)-1H-1,2,3-triazolo-[4,5-b]pyridin-1-ylmethylene]-N-methylmethanaminium hexafluorophosphate N-oxide (HATU).

General Examples for the Preparation of Compounds of the Present disclosure. The starting materials and intermediates for the compounds of this present disclosure may be prepared by the application or adaptation of the methods described below, their obvious chemical equivalents, or, for example, as described in literature such as The Science of Synthesis, Volumes 1-8. Editors E. M. Carreira et al. Thieme publishers (2001-2008). Details of reagent and reaction options are also available by structure and reaction searches using commercial computer search engines such as Scifinder (www.cas.org) or Reaxys (www.reaxys.com).

Part I: Preparation of Intermediates

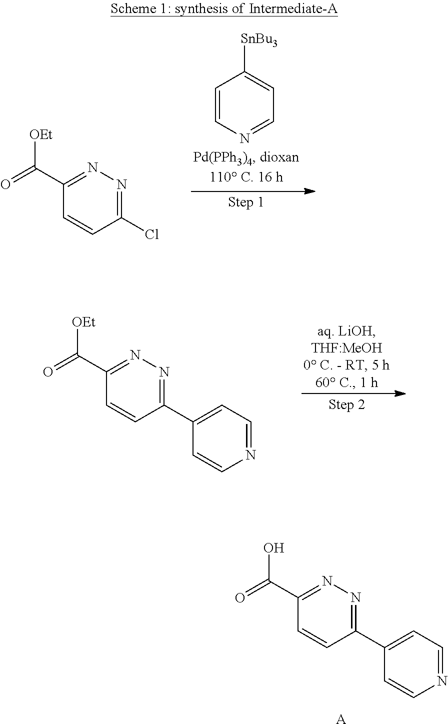

Step 1: Synthesis of ethyl 6-(pyridin-4-yl) pyridazine-3-carboxylate: To a argon-purged solution of ethyl 6-chloropyridazine-3-carboxylate (4.0 g, 21.4 mmol) was added 4-(tributylstannyl)pyridine (8.71 g, 23.65 mmol) in 1,4-dioxane (40 mL) and the resulting mixture was stirred at room temperature for 10 min before Pd(PPh₃)₄ (2.48 g, 2.15 mmol) was added. The reaction mixture was stirred at 110° C. for 16 hours. After completion, the reaction mixture was diluted with saturated aq. solution of NaHCO₃ (50 mL) solution and extracted with EtOAc (30 mL×3), dried over anhydrous Na₂SO₄, filtered and concentrated under reduced pressure. The obtained residue was purified by column chromatography to afford ethyl 6-(pyridin-4-yl)pyridazine-3-carboxylate (2.5 g, 46% yield) as a white solid. ¹H NMR (400 MHz, DMSO-d₆): δ 8.84 (m, 2H), 8.58 (d, J=8.8 Hz, 1H), 8.38 (d, J=8.8 Hz, 1H), 8.21 (m, 2H), 4.48 (q, J=7.2 Hz, 2H), 1.40 (t, J=7.2 Hz, 3H). LC-MS (ESI+): m/z; 230.14 [M+H]⁺.

Step 2: Synthesis of 6-(pyridin-4-yl) pyridazine-3-carboxylic acid (A): An aqueous solution of lithium hydroxide monohydrate (0.55 g, 13.1 mmol) in water (10 mL) was added to a solution of ethyl 6-(pyridin-4-yl)pyridazine-3-carboxylate (2.5 g, 10.9 mmol) in THF (10 mL) at 0° C. and the resulting mixture was stirred at room temperature for 5 hours. MeOH (10 mL) was added and the mixture was stirred at 60° C. for 1 h. After completion of the reaction, THF and MeOH were removed under reduced pressure and the aqueous layer was acidified with 2N HCl (pH-4). The obtained solid was filtered, washed with water and dried. Then, it was triturated with acetonitrile, filtered and the filter cake was dried to afford compound A (1.4 g, 53% yield) as a pale brown solid. ¹H NMR (400 MHz, DMSO-d₆): δ 14.02 (s, 1H), 8.84 (m, 2H), 8.56 (d, J=8.8 Hz, 1H), 8.36 (d, J=8.8 Hz, 1H), 8.21 (m, 2H). LC-MS (ESI−): m/z; 200.11 [M−H]⁻.

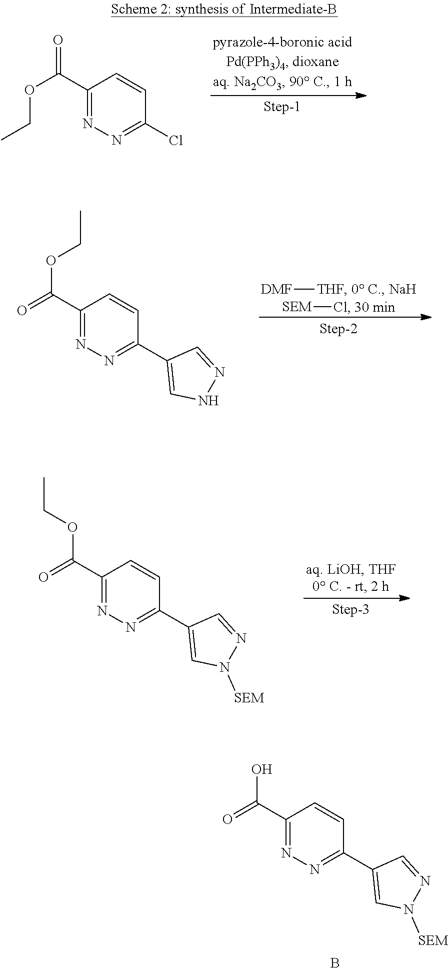

Step 1: Synthesis of ethyl 6-(1H-pyrazol-4-yl) pyridazine-3-carboxylate: Argon gas was purged through a solution of pyrazole-4-boronic acid (4.51 g, 40.31 mmol), Na₂CO₃ (7.1 g, 67.2 mmol) and ethyl 6-chloropyridazine-3-carboxylate (5 g, 26.88 mmol) in 1,4-dioxane (175 mL) and water (25 mL) for 10 mins before addition of Pd (PPh₃)₄ (1.55 g, 1.34 mmol). The reaction mixture was stirred at 90° C. for 1 h. After completion of the reaction, it was cooled to room temperature and diluted with EtOAc (250 mL). It was then washed with water (100 mL), brine (100 mL), dried over anhydrous sodium sulfate, filtered and concentrated under reduced pressure. The residue was purified by column chromatography over silica gel to afford 3.2 g of ethyl 6-(1H-pyrazol-4-yl) pyridazine-3-carboxylate as an off-white solid. LC-MS (ESI+): m/z; 219.0 [M+H]⁻.

Step 2: Synthesis of ethyl 6-(1-((2-(trimethylsilyl) ethoxy) methyl)-1H-pyrazol-4-yl)pyridazine-3-carboxylate:

NaH (60% w/w) (0.422 g, 17.6 mmol) was added portion wise to a stirred solution of ethyl 6-(1H-pyrazol-4-yl) pyridazine-3-carboxylate (3.2 g, 14.67 mmol) in THF (64 mL) and DMF (30 mL) at 0° C. and stirred for 10 mins. To this was added SEM-C1 (2.93 g, 17.61 mmol) and the reaction mixture was stirred at 0° C. for 30 min. It was then quenched with 10% citric acid solution and the solid thus obtained was filtered, washed with water (5 mL×2) and dried. The residue was purified by column chromatography over silica gel (using 0-5% Methanol in Dichloromethane as an eluent) to afford 2.65 g of ethyl 6-(1-((2-(trimethylsilyl) ethoxy) methyl)-1H-pyrazol-4-yl)pyridazine-3-carboxylate as off white solid. LC-MS (ESI+): m/z; 349.1 [M−H]⁺.

Step 3: Synthesis of 6-(1-((2-(trimethylsilyl)ethoxy)methyl)-1H-pyrazol-4-yl)pyridazine-3-carboxylic acid (B):

An aqueous solution of lithium hydroxide monohydrate (0.382 g, 9.13 mmol, in 3 mL water) was added to a solution of ethyl 6-(1-((2-(trimethylsilyl) ethoxy) methyl)-1H-pyrazol-4-yl)pyridazine-3-carboxylate (2.65 g, 7.61 mmol) in THF (9 mL) at 0° C. and stirred at room temperature for 2 h. After completion of the reaction, the reaction mixture was diluted with water (10 mL) and washed with EtOAc (30 mL×2). The aqueous layer was acidified using 2N HCl (pH-4) solution and the solid thus obtained was filtered, washed with water (2 mL×2) and dried to afford 1.1 g of B as an off-white solid. ¹H NMR (400 MHz, DMSO-d₆) δ 13.62 (s, 1H), 8.78 (s, 1H), 8.33 (s, 1H), 8.18-8.13 (m, 2H), 5.51 (s, 2H), 3.61 (t, J=8.0 Hz, 2H), 0.87 (d, J=8.0 Hz, 2H), 0.04 (s, 9H). LC-MS (ESI+): m/z 321.0 [M+H]⁺.

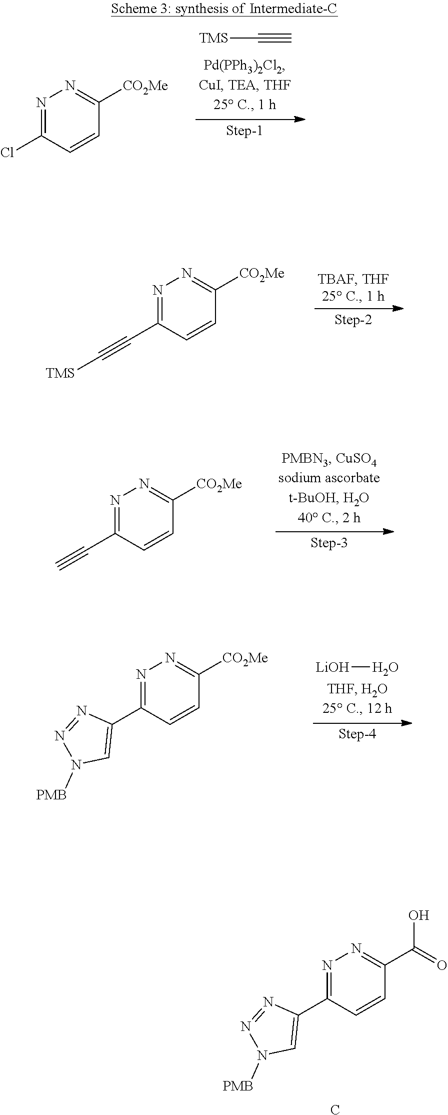

Step 1: Synthesis of methyl 6-((trimethylsilyl)ethynyl)pyridazine-3-carboxylate: To a solution of methyl 6-chloropyridazine-3-carboxylate (1 g, 5.79 mmol) in THF (10 mL) was added ethynyl(trimethyl)silane (4.0 mL, 29.0 mmol), Pd(PPh₃)₂Cl₂ (407 mg, 0.58 mmol), CuI (221 mg, 1.2 mmol) and Et₃N (0.807 mL, 5.79 mmol), and the resulting mixture was stirred at 25° C. for 1 hour. After completion of the reaction, the mixture was filtered through a pad of silica-gel and the filtrate was concentrated under reduced pressure. The residue was purified by column chromatography (PE/EtOAc) to afford methyl 6-((trimethylsilyl)ethynyl)pyridazine-3-carboxylate (500 mg, 37% yield) as a yellow solid.

Step 2: Synthesis of methyl 6-ethynylpyridazine-3-carboxylate: To a solution of methyl 6-((trimethylsilyl)ethynyl)pyridazine-3-carboxylate (500 mg, 2.13 mmol) in THF (10 mL) was added TBAF (1M in THF, 4.27 mL, 4.27 mmol), the reaction mixture was stirred at room temperature for 1 hour. After completion, the reaction mixture was poured into H₂O (50 mL) and extracted with DCM (30 mL×3). The combined organic layers were washed with brine (50 mL), dried over Na₂SO₄, filtered and concentrated under reduced pressure. The residue was purified by column chromatography (PE/EtOAc) to afford methyl 6-ethynylpyridazine-3-carboxylate (260 mg, 75% yield) as a brown solid.

Step 3: Synthesis of methyl 6-(1-(4-methoxybenzyl)-1H-1,2,3-triazol-4-yl)pyridazine-3-carboxylate: To a solution of methyl 6-ethynylpyridazine-3-carboxylate (500 mg, 3.1 mmol) and 1-(azidomethyl)-4-methoxy-benzene (1.0 g, 6.2 mmol) in H₂O (4 mL) and t-BuOH (16 mL) was added CuSO₄ (98.4 mg, 0.62 mmol) and sodium ascorbate (489 mg, 2.5 mmol). The reaction mixture was purged with nitrogen and stirred at 40° C. for 2 h. After completion of the reaction, it was diluted with EtOAc (50 mL) and H₂O (20 mL). The precipitate was filtered, and the filter cake was washed with DCM/MeOH 10/1 (500 mL). The filtrate was concentrated under reduced pressure to afford methyl 6-(1-(4-methoxybenzyl)-1H-1,2,3-triazol-4-yl)pyridazine-3-carboxylate (600 mg, 60% yield) as a gray solid. LCMS (ESI+): m/z 325.9 [M+H]⁺.

Step 4: Synthesis of lithium 6-(1-(4-methoxybenzyl)-1H-1,2,3-triazol-4-yl)pyridazine-3-carboxylate (C): To a solution of methyl 6-(1-(4-methoxybenzyl)-1H-1,2,3-triazol-4-yl)pyridazine-3-carboxylate (250 mg, 0.77 mmol) in THF (2.5 mL) was added a solution of lithium hydroxide monohydrate (96.7 mg, 2.3 mmol) in water (2.5 mL) at 0° C. After stirred at room temperature for 12 h, the precipitate was filtered, and the filter cake was dried under reduced pressure. The residue was triturated with acetonitrile and filtered to afford the acid C (70.0 mg, 29% yield) as a gray solid. LCMS (ESI+): m/z 312 [M+H]⁺.

Part II: Preparation of Example Compounds

All compounds were prepared using the procedures exemplified below.

### Example 1

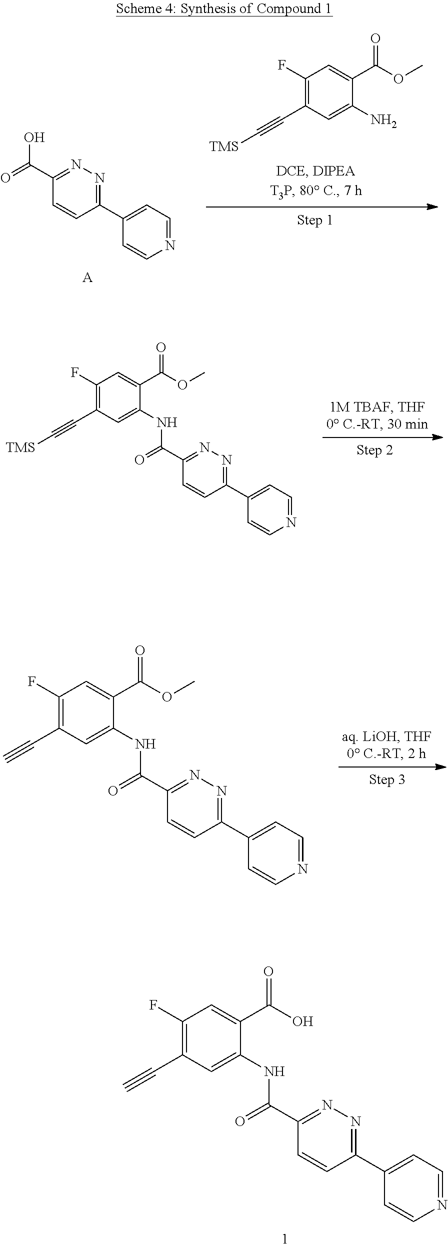

Step 1: Synthesis of methyl 5-fluoro-2-(6-(pyridin-4-yl) pyridazine-3-carboxamido)-4-((trimethylsilyl) ethynyl) benzoate:

To a solution of intermediate C (1.4 g, 7.0 mmol) and DIPEA (6.17 mL, 34.8 mmol) in DCE (30 mL) was added T₃P (50% in EtOAc) (13.29 mL, 20.89 mmol) at room temperature, followed by methyl 2-amino-5-fluoro-4-((trimethylsilyl)ethynyl)benzoate (1.8 g, 7.0 mmol). The reaction mixture was stirred at 80° C. for 7 h. After completion of reaction, the volatiles were removed under reduced pressure and saturated aq. solution of NaHCO₃ (15 mL) was added. The obtained solid was filtered, washed with water and dried. The residue was purified by column chromatography (PE/EtOAc) to afford methyl 5-fluoro-2-(6-(pyridin-4-yl) pyridazine-3-carboxamido)-4-((trimethylsilyl) ethynyl) benzoate (2.2 g, 70% yield) as a pale cream solid. ¹H NMR (400 MHz, DMSO-d₆): δ 12.96 (s, 1H), 8.98-8.84 (m, 1H), 8.69 (d, J=8.8 Hz, 2H), 8.52 (m, 1H), 8.26 (m, 1H), 8.24 (m, 2H), 7.92 (m, 1H), 3.97 (s, 3H), 0.29 (s, 9H). LC-MS (ESI−): m/z; 447.28 [M−H]⁻.

Step 2: Synthesis of methyl 4-ethynyl-5-fluoro-2-(6-(pyridin-4-yl) pyridazine-3-carboxamido) benzoate: TBAF (1M in THF) (4.9 mL, 4.9 mmol) was added to a stirring solution of 5-fluoro-2-(6-(pyridin-4-yl) pyridazine-3-carboxamido)-4-((trimethylsilyl) ethynyl) benzoate (2.2 g, 4.90 mmol) in THF (22 mL) at 0° C. and the resulting mixture was stirred at room temperature for 30 min. After completion of the reaction, saturated aq. solution of NaHCO₃ (20 mL) was added. The solid was filtered, washed with water and dried. The obtained residue was purified by column chromatography (DCM/MeOH) to afford methyl 4-ethynyl-5-fluoro-2-(6-(pyridin-4-yl) pyridazine-3-carboxamido) benzoate (1.1 g, 60% yield) as pale orange solid. ¹H NMR (400 MHz, DMSO-d₆): δ 12.96 (s, 1H), 8.97 (d, J=6.8 Hz, 1H), 8.86-8.84 (m, 2H), 8.68 (d, J=8.8 Hz, 1H), 8.51 (d, J=8.8 Hz, 1H), 8.26-8.24 (m, 2H), 7.93 (d, J=10.0 Hz, 1H), 4.87 (s, 1H), 3.97 (s, 3H). LCMS: m/z 377.2 [M+H]⁺.

Step 3: Synthesis of lithium 4-ethynyl-5-fluoro-2-(6-(pyridin-4-yl) pyridazine-3-carboxamido) benzoate (1): An aqueous solution of lithium hydroxide monohydrate (33.4 mg, 0.8 mmol) in water (2 mL) was added to a solution of methyl 4-ethynyl-5-fluoro-2-(6-(pyridin-4-yl) pyridazine-3-carboxamido) benzoate (200 mg, 0.5 mmol) in THF (4 mL) at 0° C. and the resulting mixture was stirred at room temperature for 2 hours. After completion of the reaction, the obtained solid was filtered, washed with water and dried. Then, it was triturated with acetonitrile, filtered and dried to afford compound 1 as lithium salt (99 mg, 54% yield) as an off-white solid. ¹H NMR (400 MHz, DMSO-d₆): δ 8.93 (d, J=6.8 Hz, 1H), 8.85-8.83 (m, 2H), 8.61 (d, J=8.8 Hz, 1H), 8.43 (d, J=8.8 Hz, 1H), 8.25-8.24 (m, 2H), 7.79 (d, J=10.4 Hz, 1H), 4.52 (s, 1H). LC-MS (ESI+): m/z 363.2 [M+H]⁺.

Procedures analogous to those for the synthesis of compound 1 were used for the synthesis of compounds 10, 13, 16, 19, 38, 44, 49, 52, 29, 31, 33, 46, 47, 77, 54, 53, 57, 58, 63, 66, 60, 55, 56, 46, 79, 66, 67, 68, 69, 70, 71, 73, 96, 97, 98, 99, 100, 101, 102, 103, 104, 107, 108, 10, 90, 82, 88 and 81 et al.

### Example 2

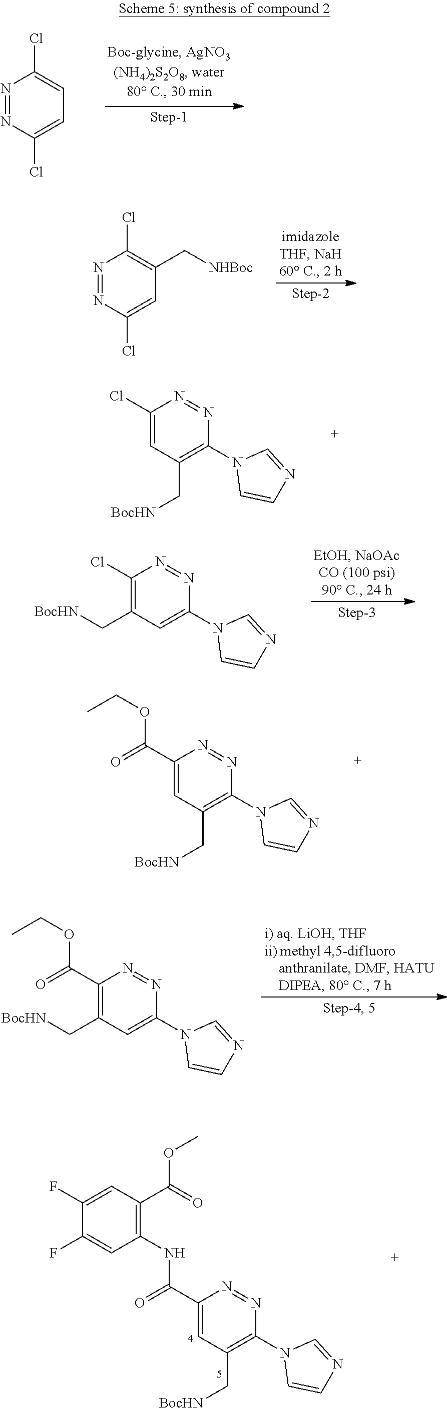

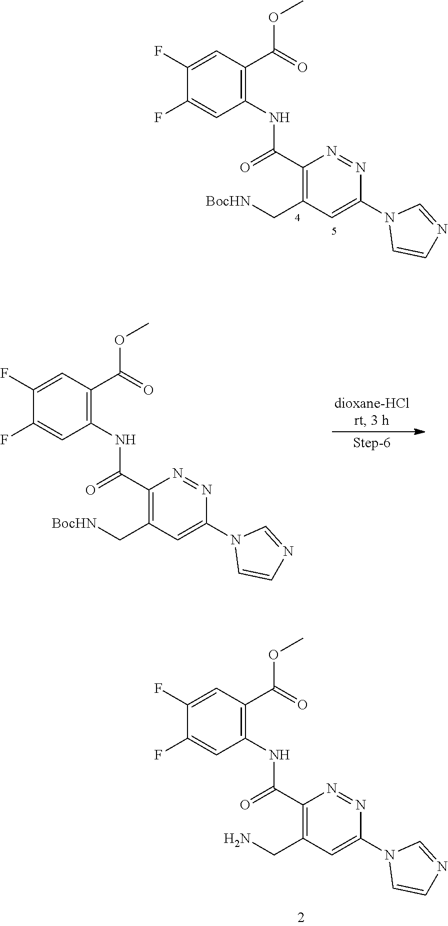 [SMILES: COC(=O)c1cc(F)c(F)cc1NC(=O)c1nnc(-n2ccnc2)cc1CNC(=O)OC(C)(C)C ; COC(=O)c1cc(F)c(F)cc1NC(=O)c1nnc(-n2ccnc2)cc1CNC(=O)OC(C)(C)C ; COC(=O)c1cc(F)c(F)cc1NC(=O)c1nnc(-n2ccnc2)cc1CN]

Step 1: Synthesis of tert-butyl ((3,6-dichloropyridazin-4-yl)methyl)carbamate: To a suspension of Boc-glycine (20.0 g, 114.2 mmol) in H₂O (100 mL) was added 3,6-dichloropyridazine (10.0 g, 67.1 mmol) and silver nitrate (1.1 g, 6.7 mmol) and the resulting mixture was heated at 80° C. To the reaction mixture was added, drop wise at 80° C. during 30 min, a solution of ammonium sulfate (27.6 g, 120.9 mmol) in H₂O (40 mL). The reaction mixture was then stirred at 80° C. for additional 30 min. Then, it was cooled to room temperature, basified with conc. ammonium hydroxide (pH 10) and extracted with EtOAc (100 mL×2). The combined organic layers were washed with brine (200 mL), dried over anhydrous Na₂SO₄ and concentrated under reduced pressure. The residue was purified by column chromatography (hexanes/EtOAc) to afford tert-butyl ((3,6-dichloropyridazin-4-yl)methyl)carbamate (15.0 g, 40% yield) as a light red thick oil. ¹H NMR (400 MHz, CDCl₃): δ 7.49 (s, 1H), 4.38 (d, J=6.0 Hz, 2H), 1.49 (s, 9H). LC-MS (ESI−): m/z 278.1 [M−H]⁻.

Step 2: Synthesis of tert-butyl ((6-chloro-3-(1H-imidazol-1-yl)pyridazin-4-yl)methyl)carbamate and tert-butyl ((3-chloro-6-(1H-imidazol-1-yl)pyridazin-4-yl)methyl)carbamate: To a solution of imidazole (5.9 g, 86.2 mmol) in THF (200 mL) was added NaH (60% in mineral oil) (3.5 g, 86.2 mmol) at 0° C. and the resulting mixture was stirred for 15 min. tert-Butyl ((3,6-dichloropyridazin-4-yl)methyl)carbamate (20.0 g, 72.1 mmol) was added and the reaction mixture was stirred at 60° C. for 2 h. After completion, the reaction was cooled to room temperature, diluted with water (200 mL), and extracted with EtOAc (200 mL×3). The combined organic layers were washed with brine (200 mL), dried over anhydrous Na₂SO₄, filtered and concentrated under reduced pressure. The residue was purified by column chromatography (PE/EtOAc), to obtain a mixture of the desired compounds (8.1 g, 36% yield) as a light brown solid, which were used in the next step as a mixture. LC-MS (ESI+): m/z r.t.=1.24 min, 310.19 [M+H]⁺ and r.t.=1.28 min, 310.15 [M+H]⁺.

Step 3: Synthesis of ethyl 5-(((tert-butoxycarbonyl)amino)methyl)-6-(1H-imidazol-1-yl)pyridazine-3-carboxylate and ethyl 4-(((tert-butoxycarbonyl)amino)methyl)-6-(1H-imidazol-1-yl)pyridazine-3-carboxylate: To a solution of a mixture of compounds tert-butyl ((6-chloro-3-(1H-imidazol-1-yl)pyridazin-4-yl)methyl)carbamate and tert-butyl ((3-chloro-6-(1H-imidazol-1-yl)pyridazin-4-yl)methyl)carbamate (6.5 g, 21.0 mmol) in EtOH (97.5 mL) was added sodium acetate (3.4 g, 41.9 mmol) and the resulting mixture was purged with argon for 10 min. Then, Pd(dppf)Cl₂ (0.77 g, 1.0 mmol) was added and the reaction mixture was stirred under CO pressure (100 psi) at 90° C. for 24 h. Then it was cooled to room temperature and volatiles were evaporated under reduced pressure. Saturated aq. solution of NaHCO₃ (100 mL) was added and extracted with EtOAc (100 mL×3). The combined organic layers were washed with brine (100 mL), dried over anhydrous Na₂SO₄, filtered and concentrated under reduced pressure. The residue was purified by column chromatography (DCM/MeOH) to afford a mixture of ethyl 5-(((tert-butoxycarbonyl)amino)methyl)-6-(1H-imidazol-1-yl)pyridazine-3-carboxylate and ethyl 4-(((tert-butoxycarbonyl)amino)methyl)-6-(1H-imidazol-1-yl)pyridazine-3-carboxylate (6.5 g, 89% yield) as a brown solid. LC-MS: m/z r.t.=1.36 min, 348.4 [M+H]⁺ and r.t.=1.29 min, 348.3 [M+H]⁺.

Step 4 and 5: Synthesis of methyl 2-(5-(((tert-butoxycarbonyl)amino)methyl)-6-(1H-imidazol-1-yl)pyridazine-3-carboxamido)-4,5-difluorobenzoate and methyl 2-(4-(((tert-butoxycarbonyl)amino)methyl)-6-(1H-imidazol-1-yl)pyridazine-3-carboxamido)-4,5 difluorobenzoate: An aqueous solution of lithium hydroxide monohydrate (0.32 g, 7.7 mmol) in water (12.5 mL) was added to a solution of ethyl 5-(((tert-butoxycarbonyl)amino)methyl)-6-(1H-imidazol-1-yl)pyridazine-3-carboxylate and ethyl 4-(((tert-butoxycarbonyl)amino)methyl)-6-(1H-imidazol-1-yl)pyridazine-3-carboxylate (2.5 g, 7.2 mmol) in THF (25 mL) and the resulting mixture was stirred at room temperature for 30 min. After completion of the reaction, THF was removed under reduced pressure and the aqueous layer was acidified with 3N HCl (pH 4-5). Volatiles were removed by lyophilization to get a mixture of the corresponding carboxylic acids. The mixture was dissolved in DMF (41 mL), and methyl 4,5-difluoroanthranilate (3.2 g, 17.1 mmol) and DIPEA (7.38 mL, 42.40 mmol) were added. To the reaction mixture, HATU (4.9 g, 12.8 mmol) was added and the reaction mixture was stirred at 80° C. for 7 hours. After completion, the reaction mixture was cooled to room temperature, diluted with saturated aq. solution of NaHCO₃ (220 mL) and extracted with EtOAc (100 mL×3). The combined organic layers were washed with brine (200 mL), dried over anhydrous Na₂SO₄, filtered and concentrated under reduced pressure. The residue was purified by column chromatography (DCM/MeOH) to afford methyl 2-(5-(((tert-butoxycarbonyl)amino)methyl)-6-(1H-imidazol-1-yl)pyridazine-3-carboxamido)-4,5-difluoro benzoate (0.75 g, 21% yield) as a yellow solid and methyl 2-(4-(((tert-butoxycarbonyl)amino)methyl)-6-(1H-imidazol-1-yl)pyridazine-3-carboxamido)-4,5-difluoro benzoate (0.11 g, 3% yield) as a fluffy light brown solid, 5-substituted compound: ¹H NMR (400 MHz, DMSO-d₆): δ 13.09 (s, 1H), 8.88-8.81 (m, 1H), 8.35 (s, 1H), 8.43 (s, 1H), 8.14-8.06 (m, 1H), 7.89-7.84 (m, 2H), 7.26 (s, 1H), 4.37 (d, J=6.0 Hz, 2H), 3.95 (s, 3H), 1.40 (s, 9H). LC-MS (ESI+): m/z 489.69 [M+H]⁺. 4-substituted compound: ¹H NMR (400 MHz, DMSO-d₆): δ 12.99 (s, 1H), 8.84-8.77 (m, 2H), 8.15-8.07 (m, 3H), 7.36 (s, 1H), 7.29 (s, 1H), 4.79 (d, J=5.6 Hz, 2H), 3.95 (s, 3H), 1.42 (s, 9H). LC-MS (ESI+): m/z 487.3 [M−H]⁻.

Step 6: Synthesis of methyl 2-(4-(aminomethyl)-6-(1H-imidazol-1-yl)pyridazine-3-carboxamido)-4,5-difluorobenzoate 2: To a solution of 2-(4-(((tert-butoxycarbonyl)amino)methyl)-6-(1H-imidazol-1-yl)pyridazine-3-carboxamido)-4,5-difluoro benzoate (600 mg, 1.2 mmol) in DCM (0.5 mL) was added 4M HCl in dioxane (5 mL) and the reaction mixture was stirred at room temperature for 3 h. After completion of the reaction, volatiles were removed under reduced pressure and diethyl ether (10 mL) was added to the residue. The obtained solid was filtered and dried to afford compound 2 (HCl salt) (34 mg, 7% yield) as an off-white solid. ¹H NMR (400 MHz, DMSO-d₆): δ 13.10 (s, 1H), 9.43 (s, 1H), 8.91 (s, 1H), 8.77-8.83 (m, 4H), 8.44 (s, 1H), 8.10-8.15 (m, 1H), 7.62 (s, 1H), 4.68 (t, J=5.2 Hz, 2H), 3.96 (s, 3H). LC-MS (ESI+): m/z 389.2 [M+H]⁺.

Compounds 7, 42, 43 and 74 were prepared by using procedures analogous to those for synthesizing compound 2.

### Example 3

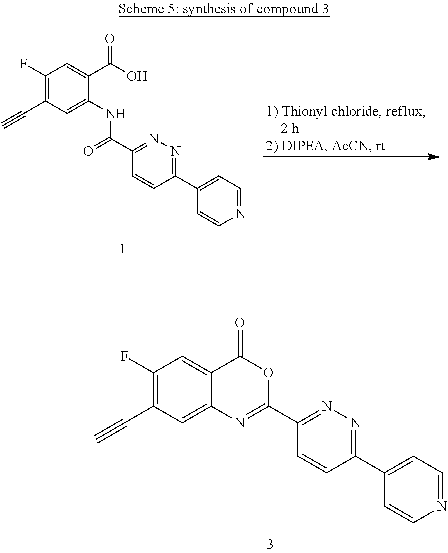 [SMILES: C#Cc1cc(NC(=O)c2ccc(-c3ccncc3)nn2)c(C(=O)O)cc1F ; C#Cc1cc2nc(-c3ccc(-c4ccncc4)nn3)oc(=O)c2cc1F]

Synthesis of 7-ethynyl-6-fluoro-2-(6-(pyridin-4-yl)pyridazin-3-yl)-4H-benzo[d][1,3]oxazin-4-one (3): A suspension of compound 1 (36 mg, 0.1 mmol) in 0.5 mL of thionyl chloride was heated under reflux for 2 h. Then, the excess thionyl was removed under vacuum. 2 mL of anhydrous acetonitrile was added the solid and a solution of DIPEA (35 μL, 0.2 mmol) in 2 mL of anhydrous acetonitrile was added at room temperature. After stirring for 30 minutes, the obtained precipitate was isolated and washed with acetonitrile to give the product (28 mg, 80% yield). ¹H NMR (400 MHz, DMSO) δ 8.91-8.82 (m, 2H), 8.63 (q, J=9.0 Hz, 2H), 8.30-8.20 (m, 2H), 8.13 (d, J=8.6 Hz, 1H), 8.08 (d, J=6.2 Hz, 1H), 5.04 (s, 1H). MS-ESI: m/z 345.46 observed (M+H)⁺

Compounds 75, 80 and 93 were prepared by using a procedure analogous to that use for synthesizing compound 3.

While the present disclosure has been described and exemplified in sufficient detail for those skilled in this art to make and use it, various alternatives, modifications, and improvements will be apparent to those skilled in the art without departing from the spirit and scope of the claims.

All patents and publications referred to herein are incorporated by reference herein to the same extent as if each individual publication was specifically and individually indicated to be incorporated by reference in its entirety.

1. A compound of formula (IA) or formula (II): 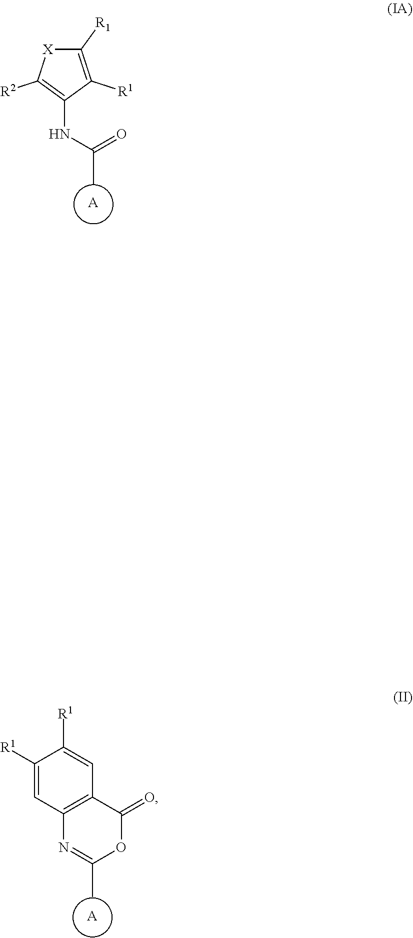
wherein
X is S, —N═C(R¹)—, or —C(R¹)═C(R¹)—;
each R¹ is independently H, F, Cl, C₁-C₆-alkyl, ethenyl or ethynyl (either of which can be substituted), cyano, alkoxyl, or haloalkyl;
R² is selected from the group consisting of —C(O)OR, —C(O)NH(C₁-C₆-alkyl) (wherein the alkyl is optionally substituted), optionally substituted C₃-C₆-cycloalkenyl, and 3- to 10-membered heterocyclyl;
R is selected from the group consisting of H, alkyl optionally substituted with —((C₁-C₆-alkyl)OC(O)OC₁-C₆-alkyl) or 3- to 10-membered heterocyclyl, and benzyl, wherein the benzyl can be unsubstituted or substituted with methoxyl or with an acid or ester isostere;
Ring A is a 5- or 6-membered heteroaryl comprising 1, 2, or 3 N atoms, unsubstituted or substituted with 1, 2, or 3 groups independently selected from the group consisting of NH₂, NH-benzyl (wherein the benzyl is unsubstituted or is substituted with methoxyl, cyano, alkylnitrile, haloalkyl, hydroxymethyl, aminomethyl, aminopropyl, carboxamido, or alkoxy), 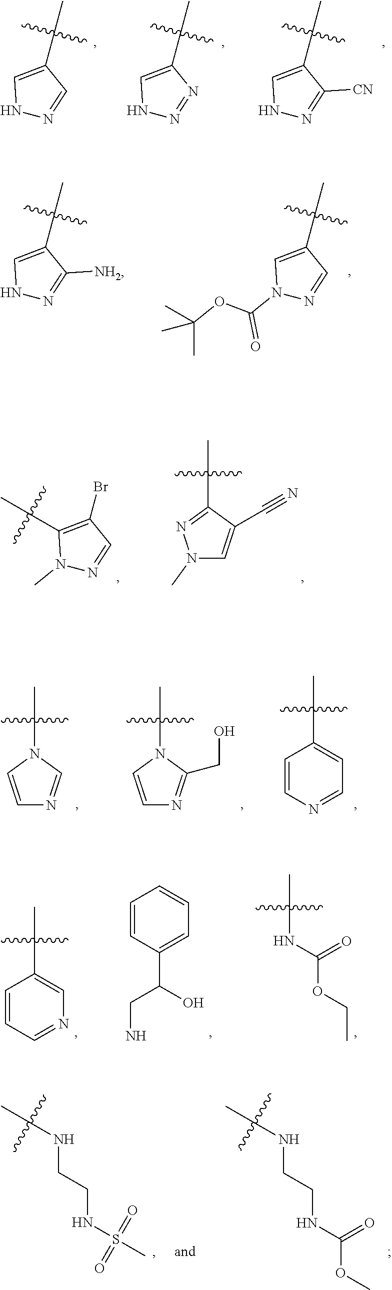
wherein a wavy line indicates a position of bonding;
or a pharmaceutically acceptable salt thereof.

2. The compound according to claim 1, wherein the compound of formula (IA) is of formula (I): 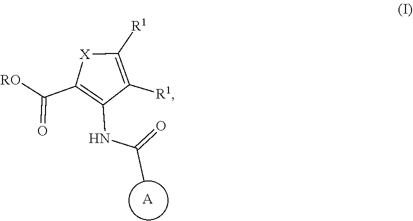
wherein
X is S, —N═C(R¹)—, or —C(R¹)═C(R¹)—;
each R¹ is independently H, F, Cl, ethenyl or ethynyl (either of which can be substituted), cyano, alkoxyl, or haloalkyl; and
R is H, alkyl, or benzyl, wherein the benzyl can be unsubstituted or substituted with methoxyl or with an acid or ester isostere.

3. The compound according to claim 1, wherein the compound is of formula (II).

4. The compound according to claim 1, wherein ring A comprises any one of pyridazinyl, triazolyl, pyrimidinyl, and pyridinyl, any of which can be unsubstituted or substituted.

5. The compound according to claim 1, wherein the compound is one selected from the following table:

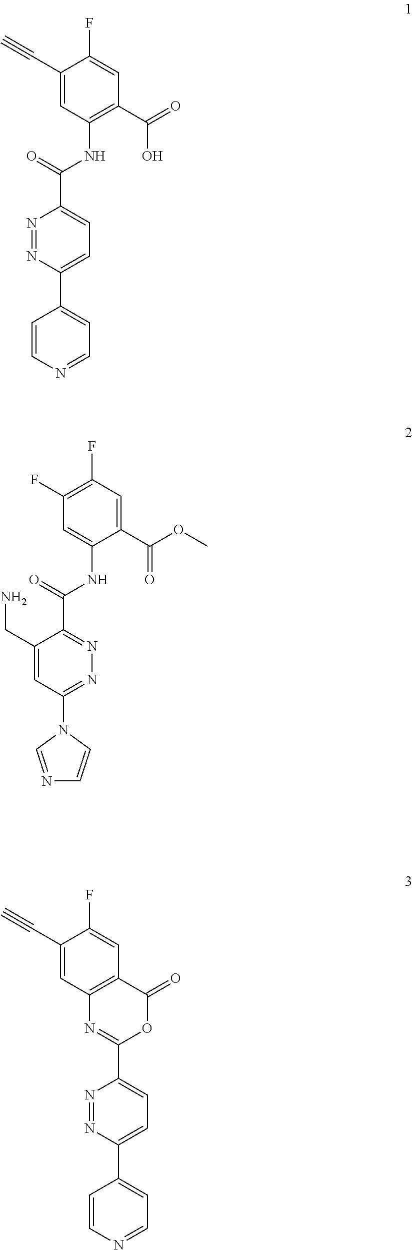

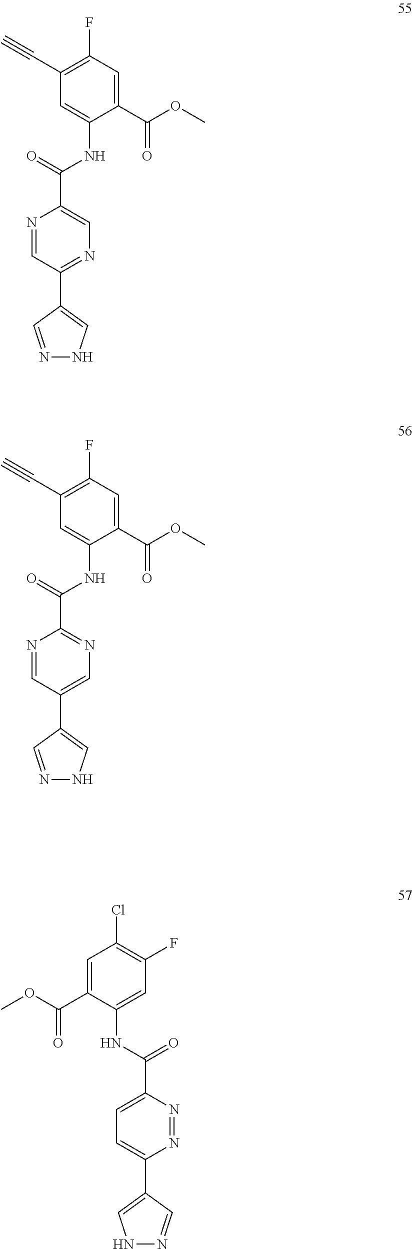

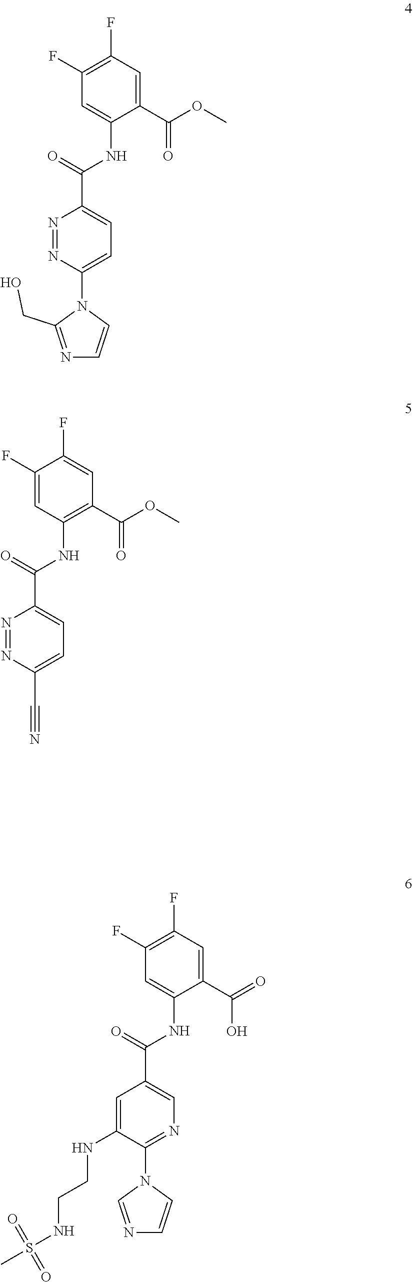

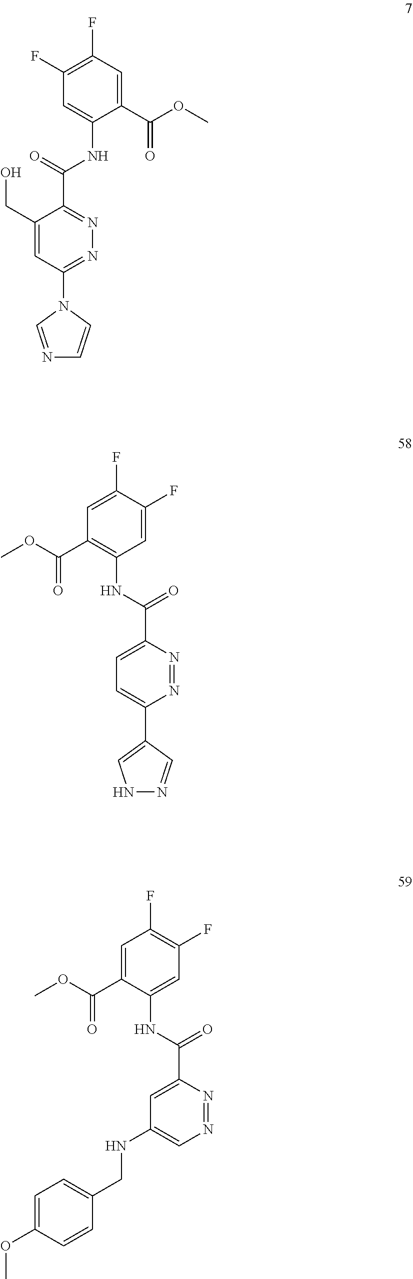

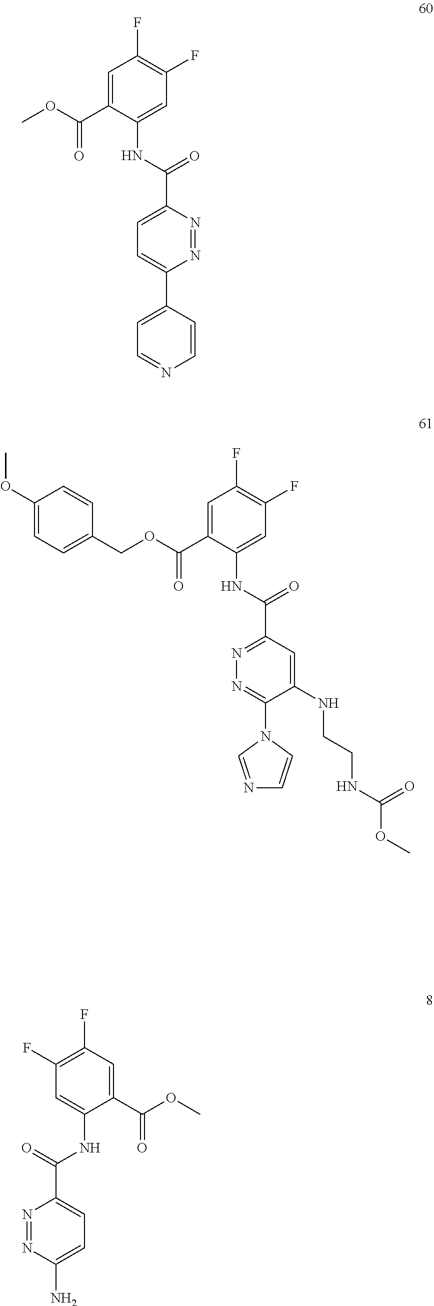 [SMILES: COC(=O)c1cc(F)c(F)cc1NC(=O)c1ccc(-c2ccncc2)nn1 ; COC(=O)NCCNc1cc(C(=O)Nc2cc(F)c(F)cc2C(=O)OCc2ccc(OC)cc2)nnc1-n1ccnc1 ; COC(=O)c1cc(F)c(F)cc1NC(=O)c1ccc(N)nn1]

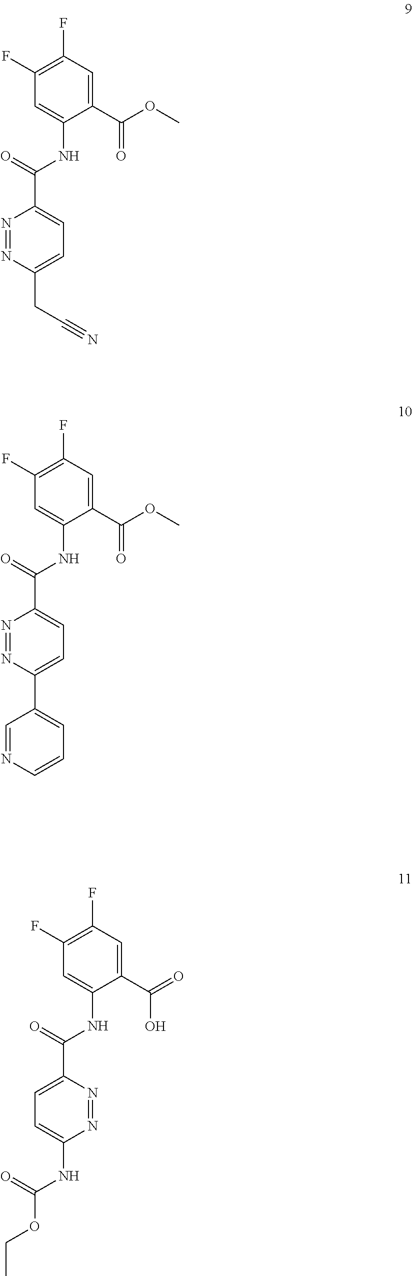 [SMILES: COC(=O)c1cc(F)c(F)cc1NC(=O)c1ccc(CC#N)nn1 ; COC(=O)c1cc(F)c(F)cc1NC(=O)c1ccc(-c2cccnc2)nn1 ; CCOC(=O)Nc1ccc(C(=O)Nc2cc(F)c(F)cc2C(=O)O)nn1]

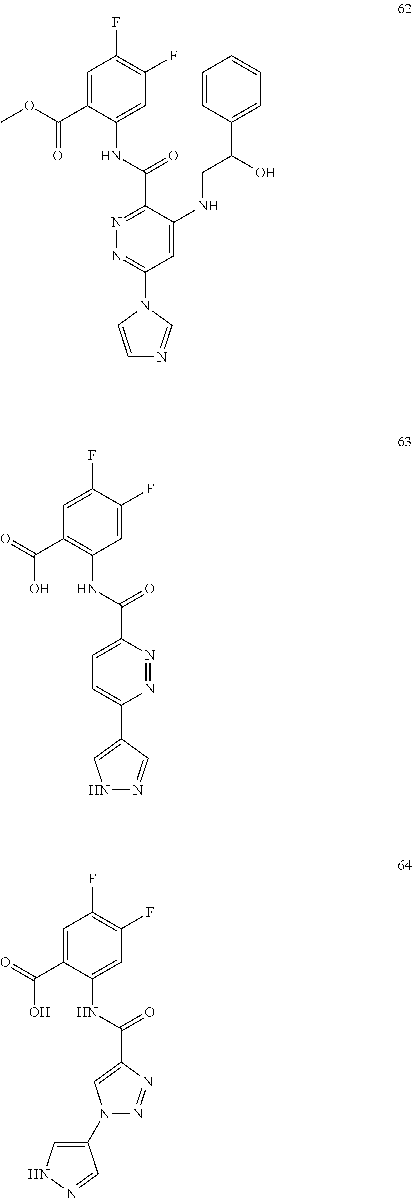 [SMILES: COC(=O)c1cc(F)c(F)cc1NC(=O)c1nnc(-n2ccnc2)cc1NCC(O)c1ccccc1 ; O=C(Nc1cc(F)c(F)cc1C(=O)O)c1ccc(-c2cn[nH]c2)nn1 ; O=C(Nc1cc(F)c(F)cc1C(=O)O)c1cn(-c2cn[nH]c2)nn1]

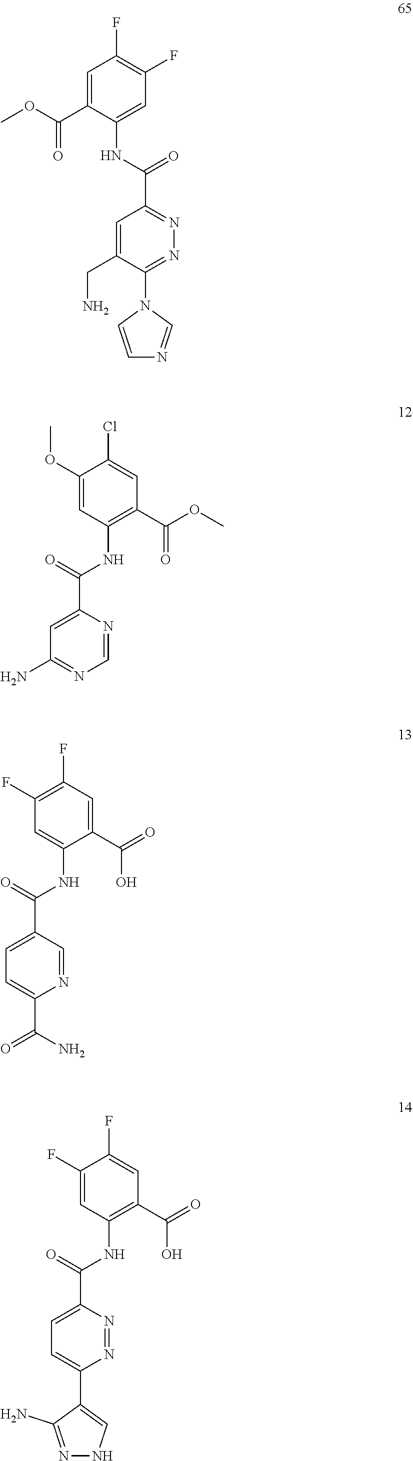 [SMILES: COC(=O)c1cc(F)c(F)cc1NC(=O)c1cc(CN)c(-n2ccnc2)nn1 ; COC(=O)c1cc(Cl)c(OC)cc1NC(=O)c1cc(N)ncn1 ; NC(=O)c1ccc(C(=O)Nc2cc(F)c(F)cc2C(=O)O)cn1 ; Nc1n[nH]cc1-c1ccc(C(=O)Nc2cc(F)c(F)cc2C(=O)O)nn1]

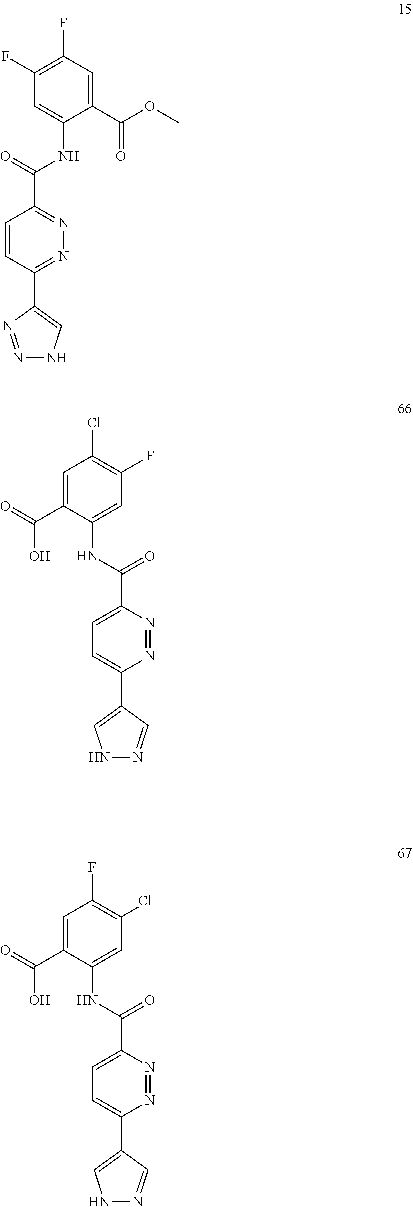 [SMILES: COC(=O)c1cc(F)c(F)cc1NC(=O)c1ccc(-c2c[nH]nn2)nn1 ; O=C(Nc1cc(F)c(Cl)cc1C(=O)O)c1ccc(-c2cn[nH]c2)nn1 ; O=C(Nc1cc(Cl)c(F)cc1C(=O)O)c1ccc(-c2cn[nH]c2)nn1]

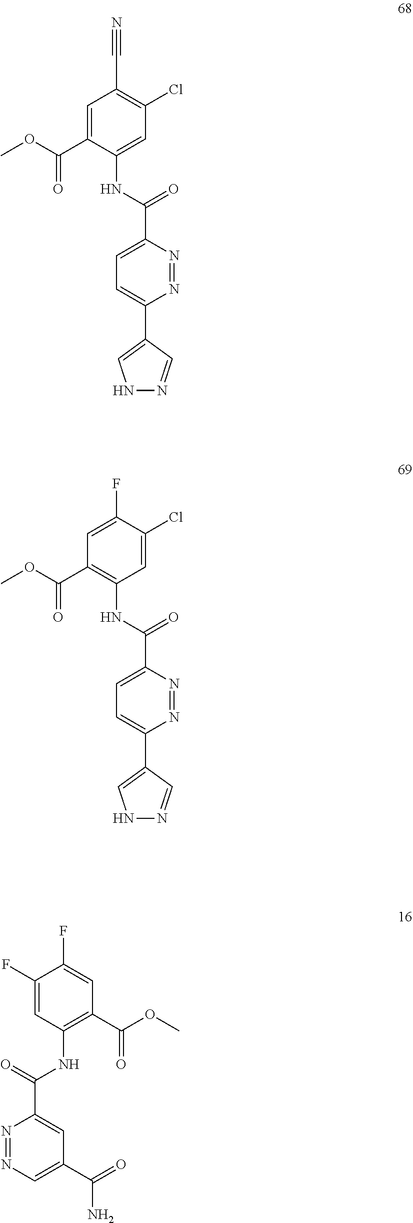 [SMILES: COC(=O)c1cc(C#N)c(Cl)cc1NC(=O)c1ccc(-c2cn[nH]c2)nn1 ; COC(=O)c1cc(F)c(Cl)cc1NC(=O)c1ccc(-c2cn[nH]c2)nn1 ; COC(=O)c1cc(F)c(F)cc1NC(=O)c1cc(C(N)=O)cnn1]

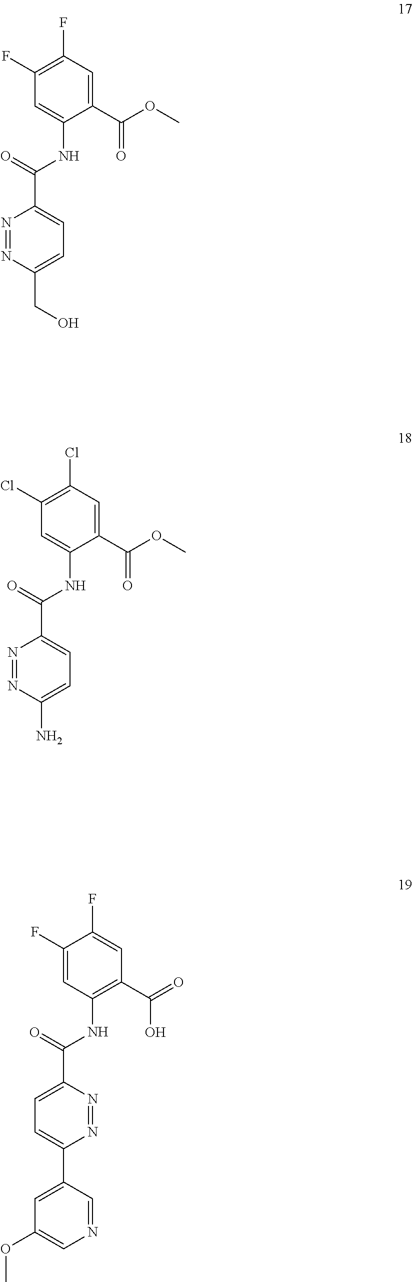 [SMILES: COC(=O)c1cc(F)c(F)cc1NC(=O)c1ccc(CO)nn1 ; COC(=O)c1cc(Cl)c(Cl)cc1NC(=O)c1ccc(N)nn1 ; COc1cncc(-c2ccc(C(=O)Nc3cc(F)c(F)cc3C(=O)O)nn2)c1]

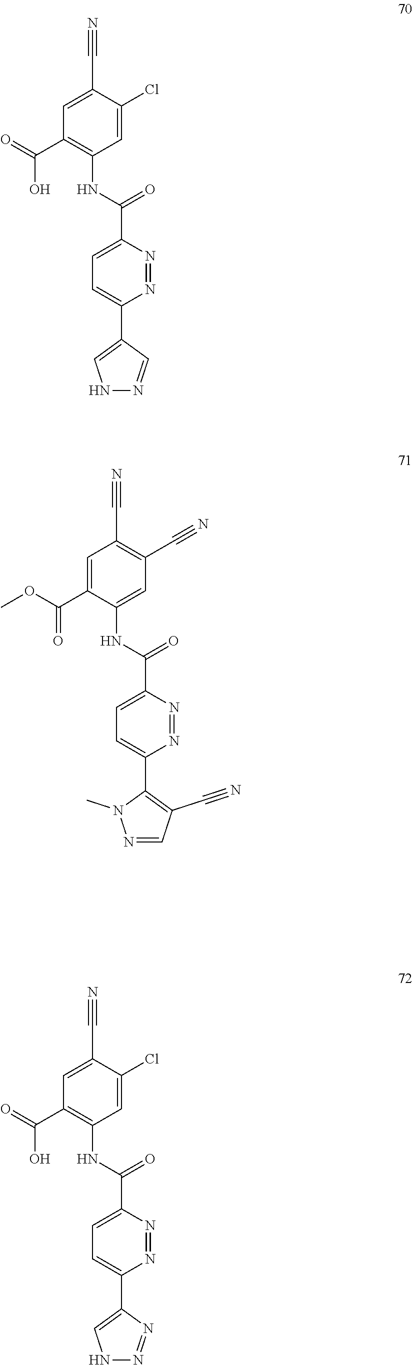 [SMILES: N#Cc1cc(C(=O)O)c(NC(=O)c2ccc(-c3cn[nH]c3)nn2)cc1Cl ; COC(=O)c1cc(C#N)c(C#N)cc1NC(=O)c1ccc(-c2c(C#N)cnn2C)nn1 ; N#Cc1cc(C(=O)O)c(NC(=O)c2ccc(-c3c[nH]nn3)nn2)cc1Cl]

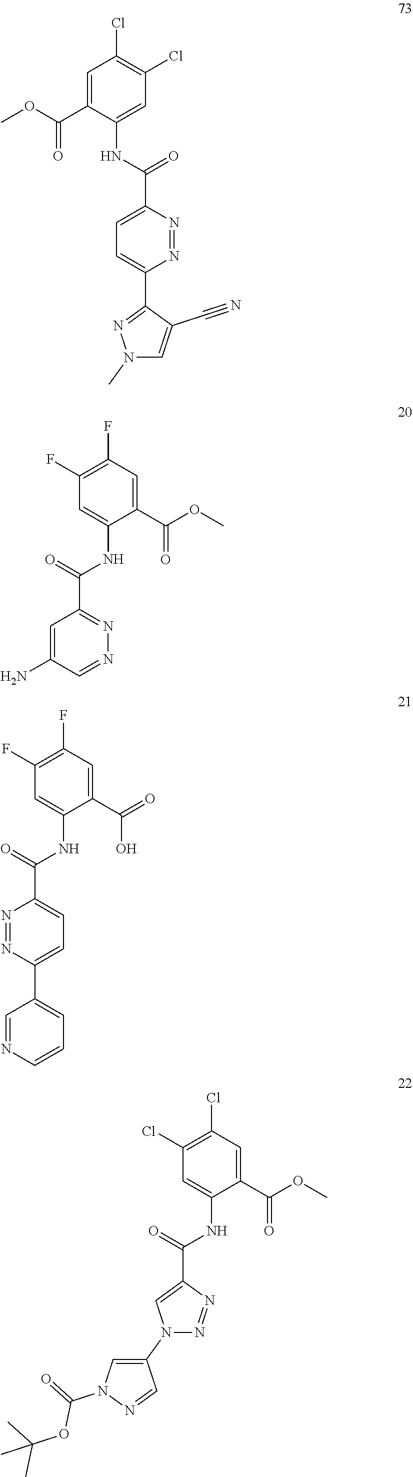

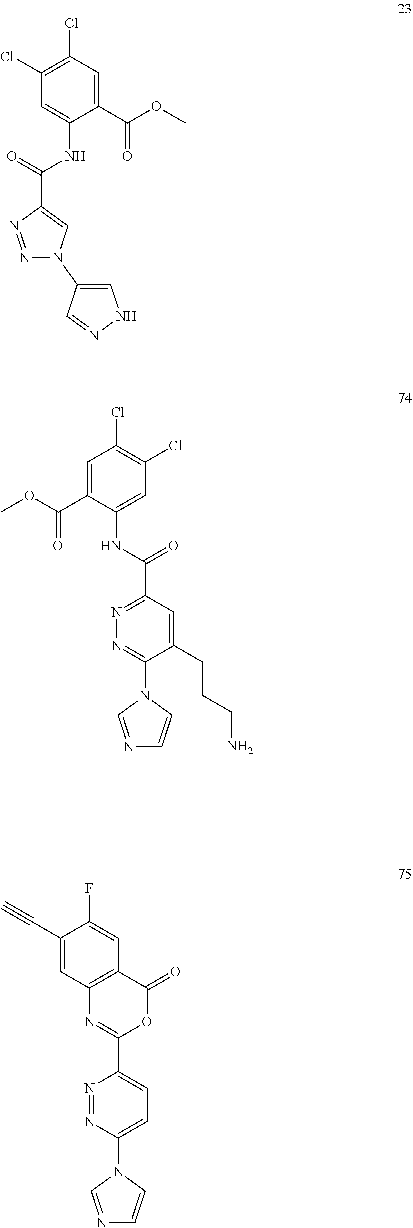

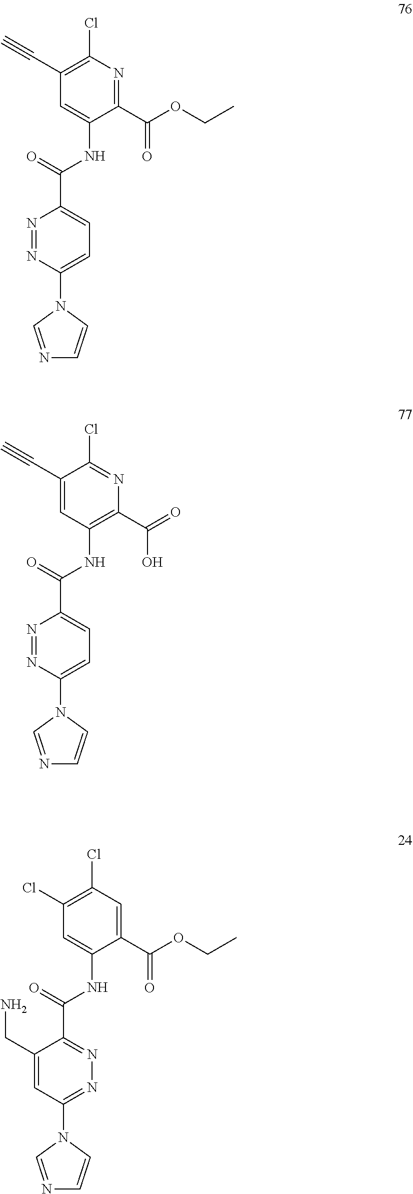

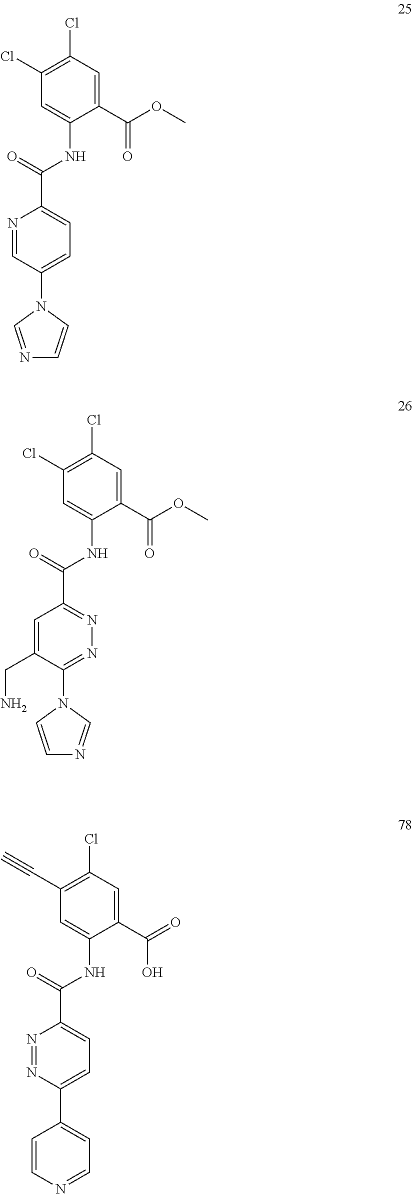

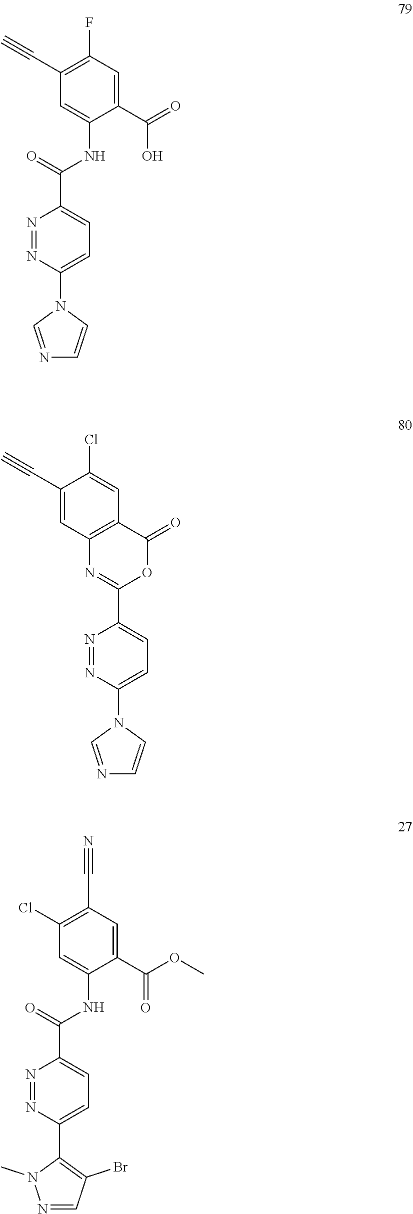

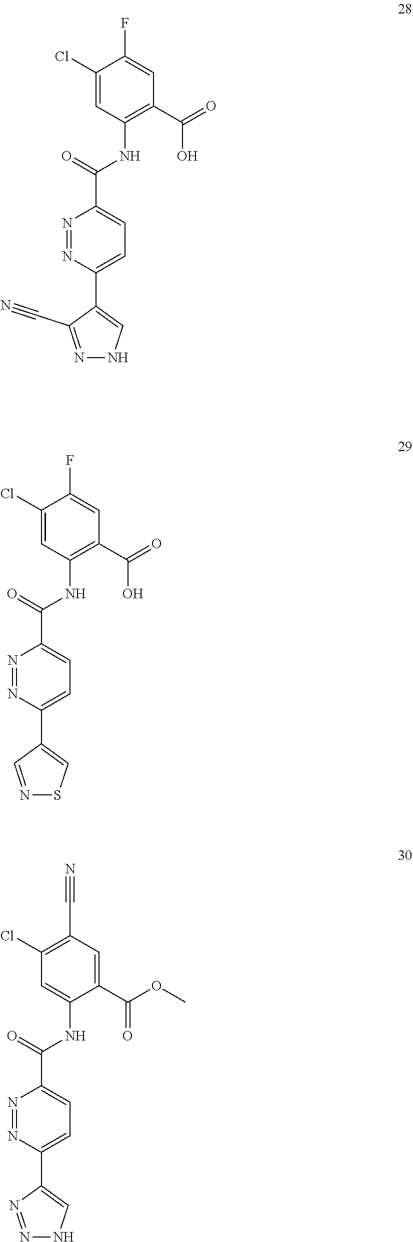 [SMILES: N#Cc1n[nH]cc1-c1ccc(C(=O)Nc2cc(Cl)c(F)cc2C(=O)O)nn1 ; O=C(Nc1cc(Cl)c(F)cc1C(=O)O)c1ccc(-c2cnsc2)nn1 ; COC(=O)c1cc(C#N)c(Cl)cc1NC(=O)c1ccc(-c2c[nH]nn2)nn1]

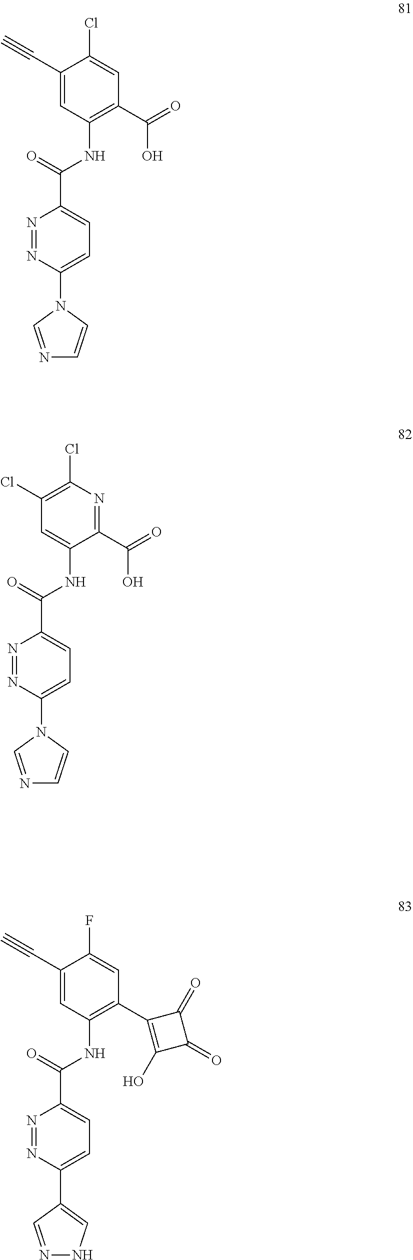 [SMILES: C#Cc1cc(NC(=O)c2ccc(-n3ccnc3)nn2)c(C(=O)O)cc1Cl ; O=C(Nc1cc(Cl)c(Cl)nc1C(=O)O)c1ccc(-n2ccnc2)nn1 ; C#Cc1cc(NC(=O)c2ccc(-c3cn[nH]c3)nn2)c(-c2c(O)c(=O)c2=O)cc1F]

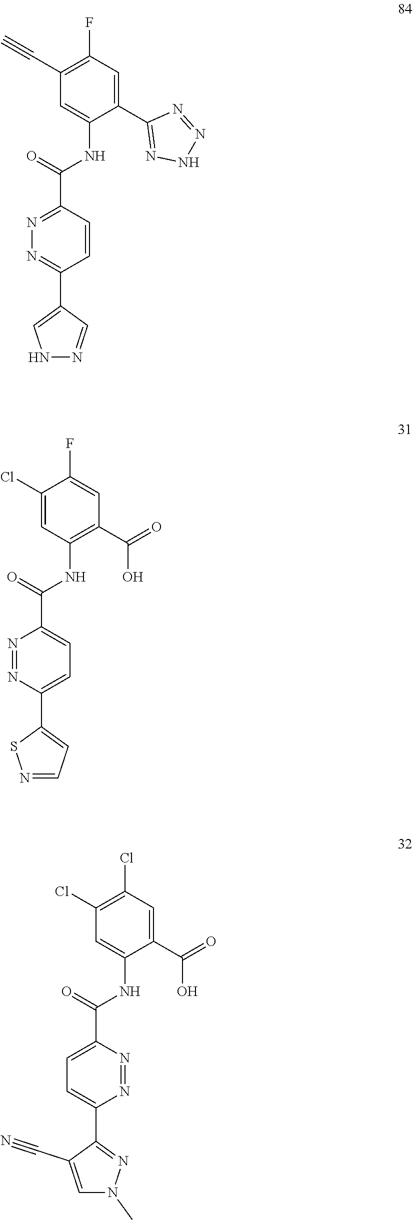 [SMILES: C#Cc1cc(NC(=O)c2ccc(-c3cn[nH]c3)nn2)c(-c2nn[nH]n2)cc1F ; O=C(Nc1cc(Cl)c(F)cc1C(=O)O)c1ccc(-c2ccns2)nn1 ; Cn1cc(C#N)c(-c2ccc(C(=O)Nc3cc(Cl)c(Cl)cc3C(=O)O)nn2)n1]

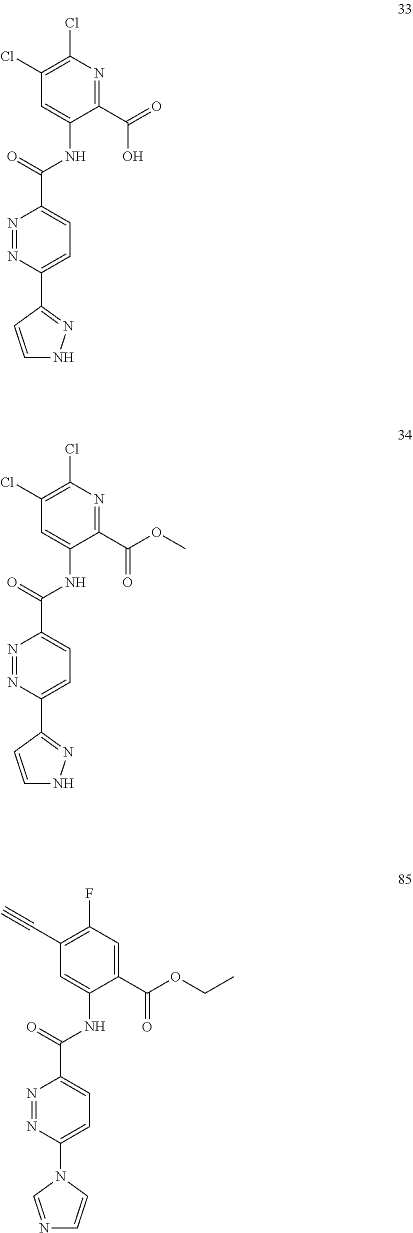

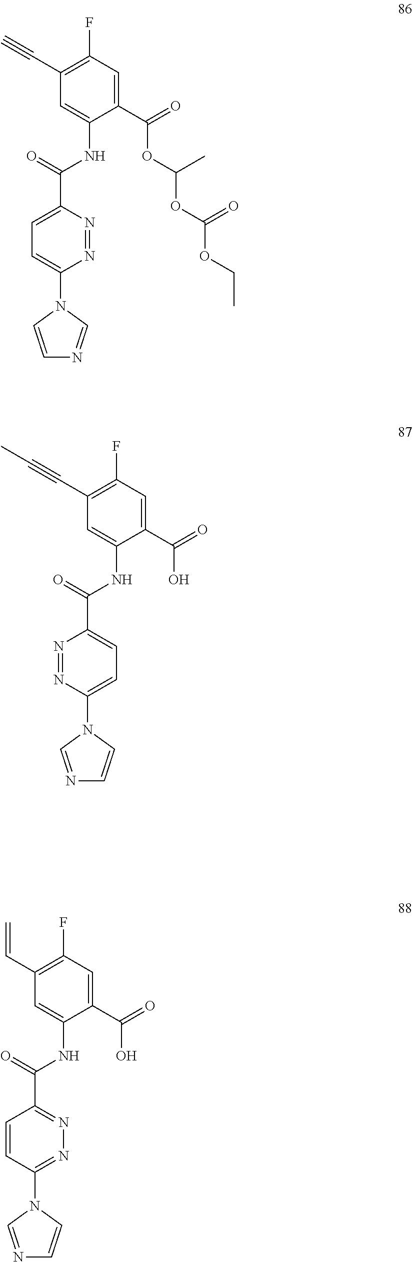

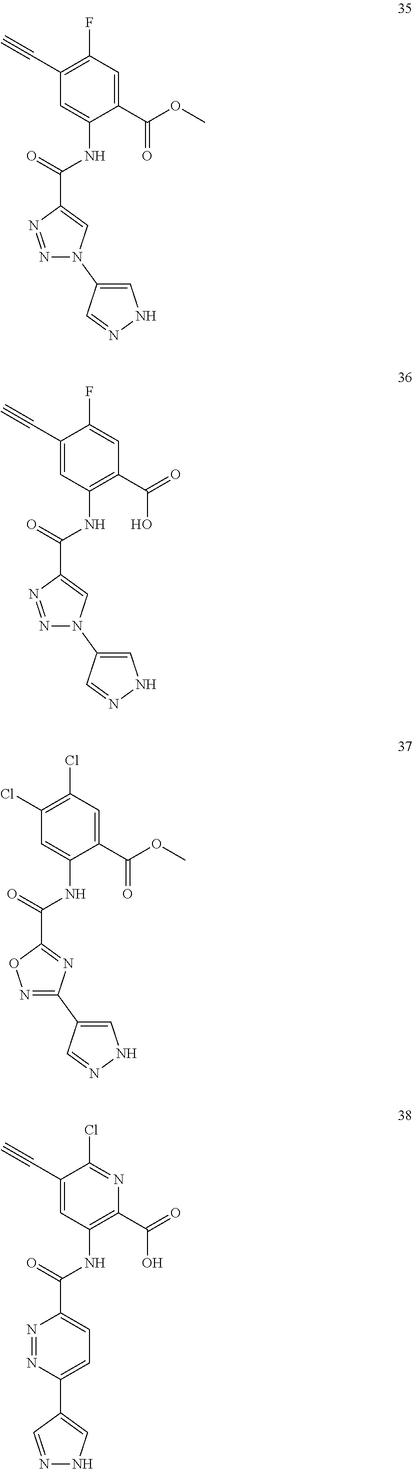

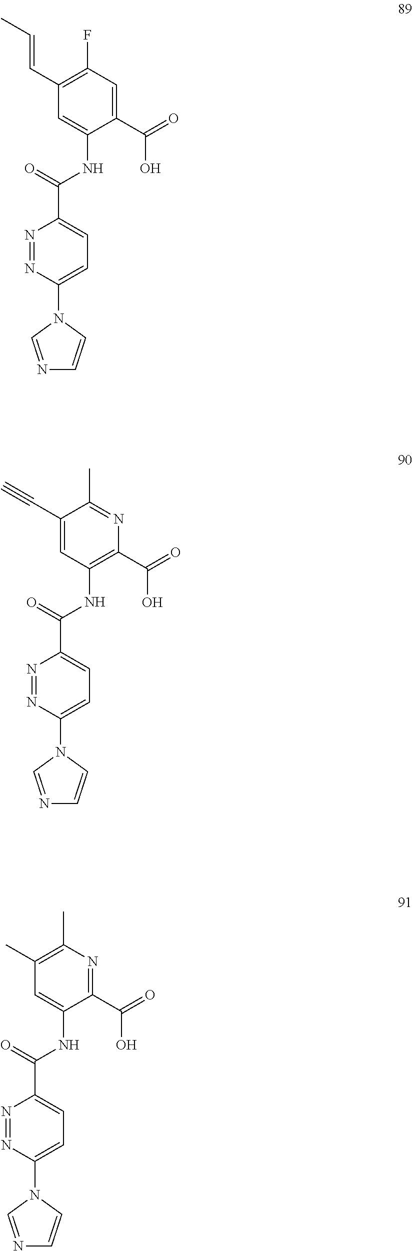 [SMILES: C/C=C/c1cc(NC(=O)c2ccc(-n3ccnc3)nn2)c(C(=O)O)cc1F ; C#Cc1cc(NC(=O)c2ccc(-n3ccnc3)nn2)c(C(=O)O)nc1C ; Cc1cc(NC(=O)c2ccc(-n3ccnc3)nn2)c(C(=O)O)nc1C]

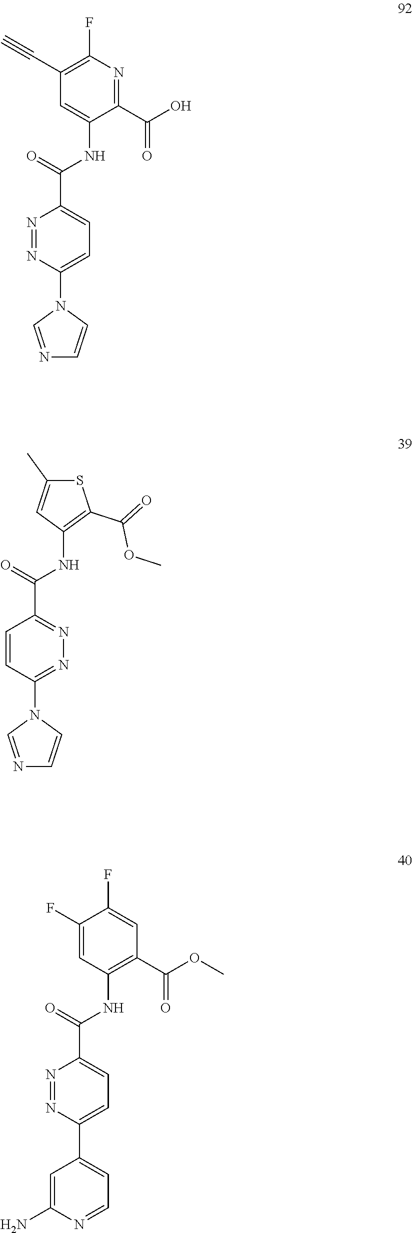 [SMILES: C#Cc1cc(NC(=O)c2ccc(-n3ccnc3)nn2)c(C(=O)O)nc1F ; COC(=O)c1sc(C)cc1NC(=O)c1ccc(-n2ccnc2)nn1 ; COC(=O)c1cc(F)c(F)cc1NC(=O)c1ccc(-c2ccnc(N)c2)nn1]

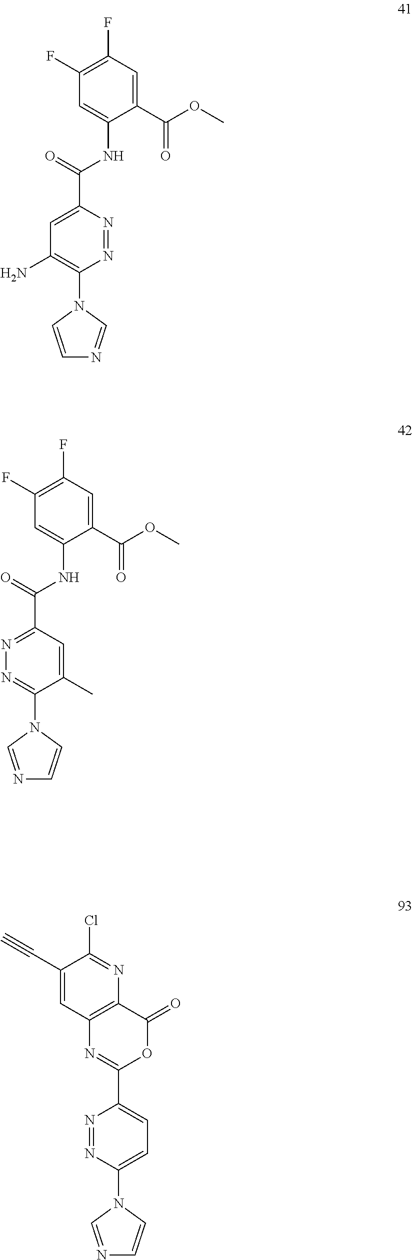

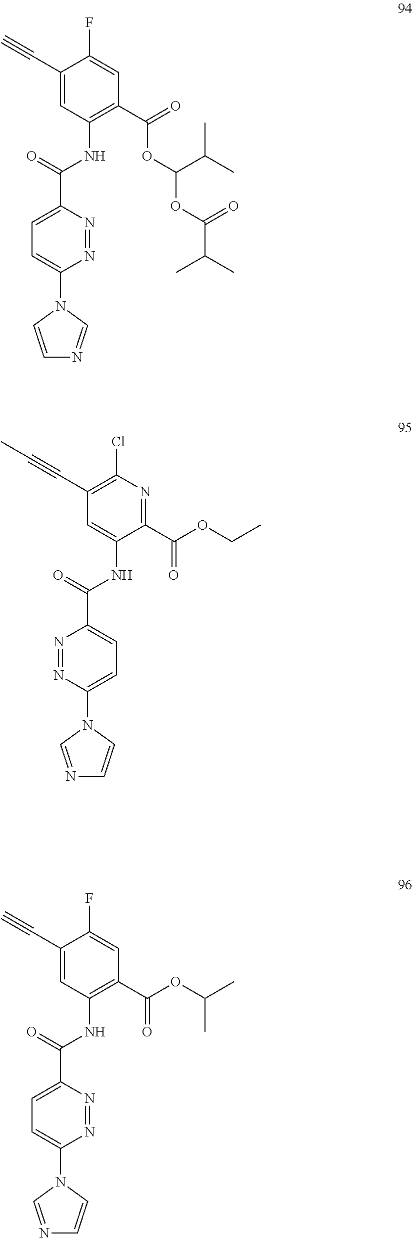

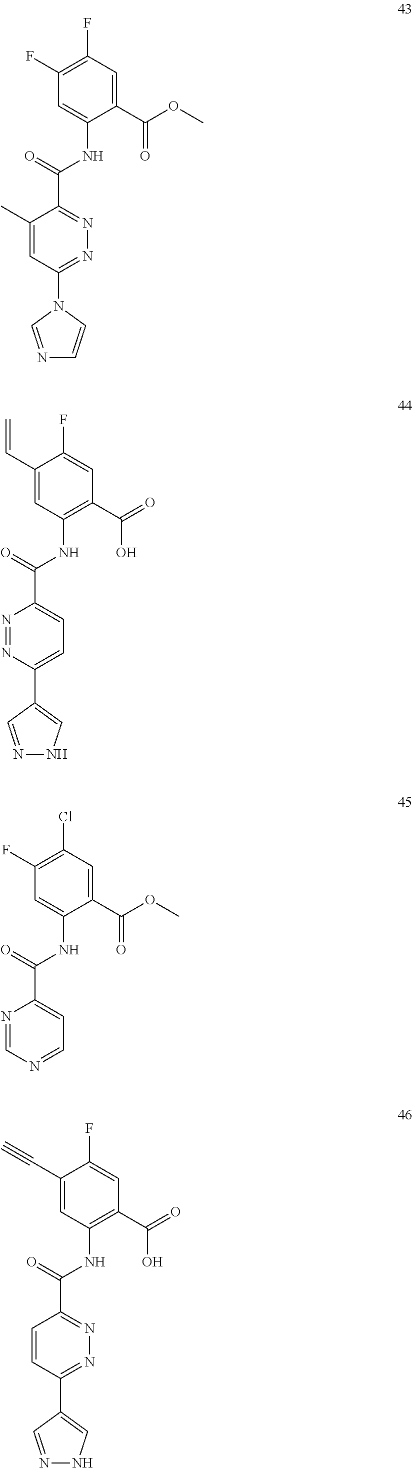

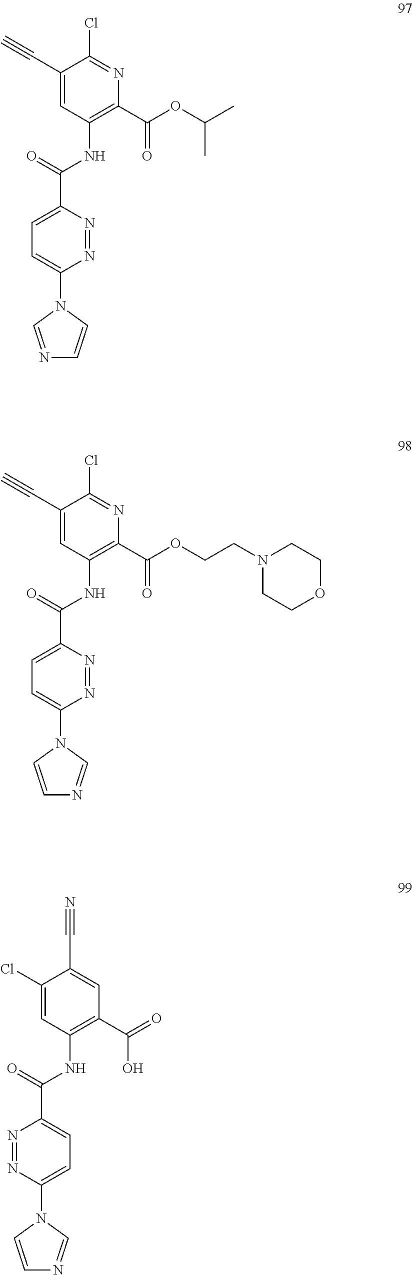

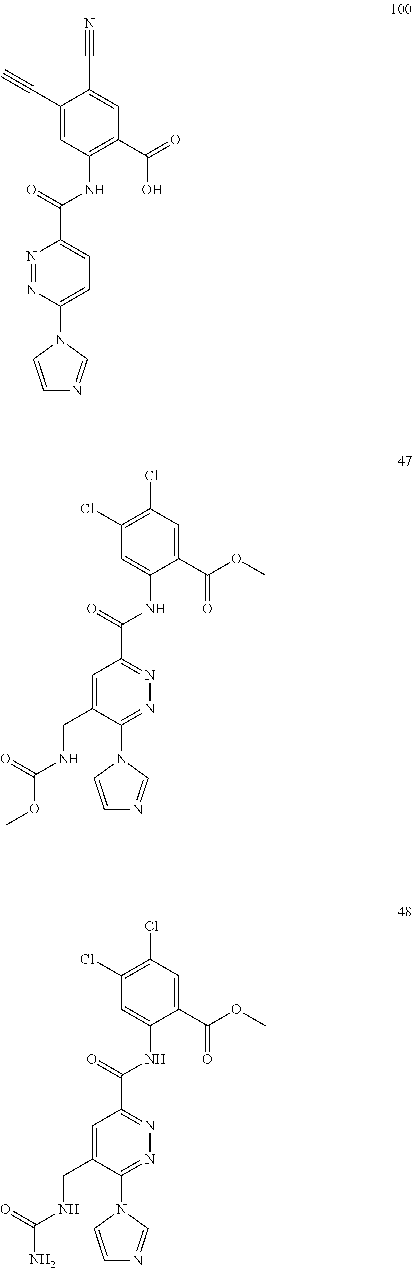 [SMILES: C#Cc1cc(NC(=O)c2ccc(-n3ccnc3)nn2)c(C(=O)O)cc1C#N ; COC(=O)NCc1cc(C(=O)Nc2cc(Cl)c(Cl)cc2C(=O)OC)nnc1-n1ccnc1 ; COC(=O)c1cc(Cl)c(Cl)cc1NC(=O)c1cc(CNC(N)=O)c(-n2ccnc2)nn1]

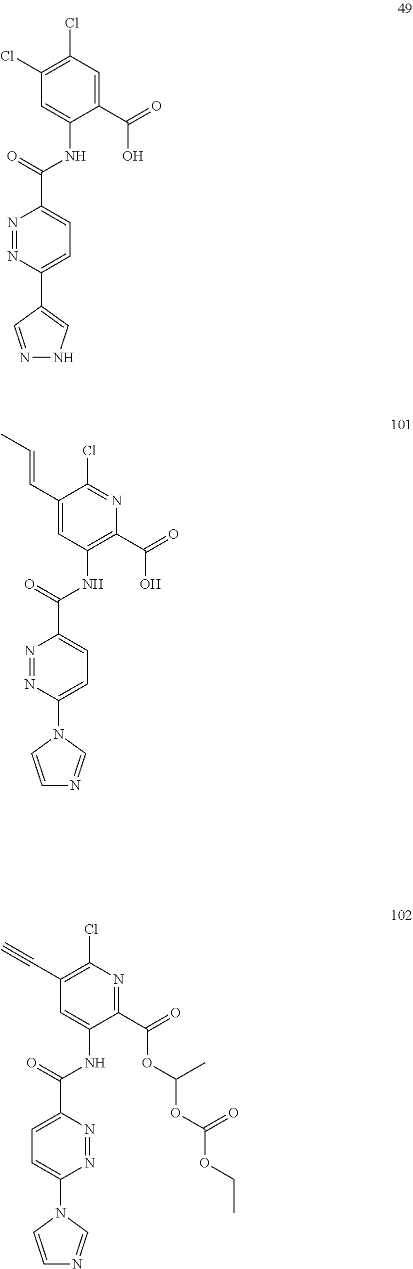 [SMILES: O=C(Nc1cc(Cl)c(Cl)cc1C(=O)O)c1ccc(-c2cn[nH]c2)nn1 ; C/C=C/c1cc(NC(=O)c2ccc(-n3ccnc3)nn2)c(C(=O)O)nc1Cl ; C#Cc1cc(NC(=O)c2ccc(-n3ccnc3)nn2)c(C(=O)OC(C)OC(=O)OCC)nc1Cl]

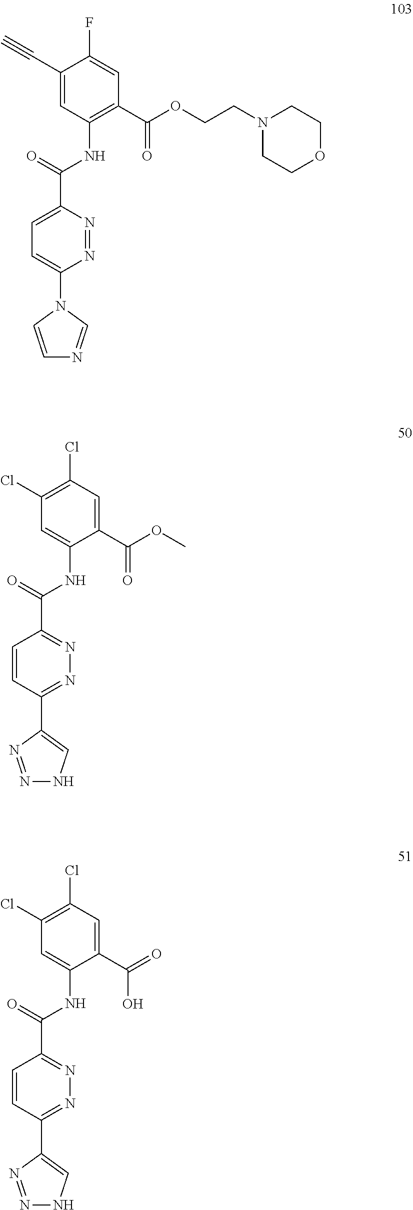 [SMILES: C#Cc1cc(NC(=O)c2ccc(-n3ccnc3)nn2)c(C(=O)OCCN2CCOCC2)cc1F ; COC(=O)c1cc(Cl)c(Cl)cc1NC(=O)c1ccc(-c2c[nH]nn2)nn1 ; O=C(Nc1cc(Cl)c(Cl)cc1C(=O)O)c1ccc(-c2c[nH]nn2)nn1]

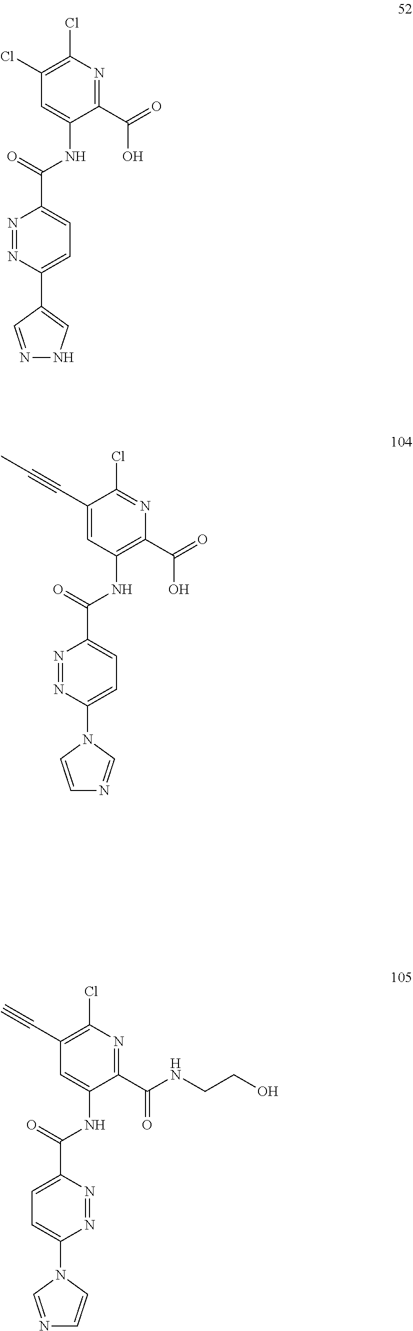 [SMILES: O=C(Nc1cc(Cl)c(Cl)nc1C(=O)O)c1ccc(-c2cn[nH]c2)nn1 ; CC#Cc1cc(NC(=O)c2ccc(-n3ccnc3)nn2)c(C(=O)O)nc1Cl ; C#Cc1cc(NC(=O)c2ccc(-n3ccnc3)nn2)c(C(=O)NCCO)nc1Cl]

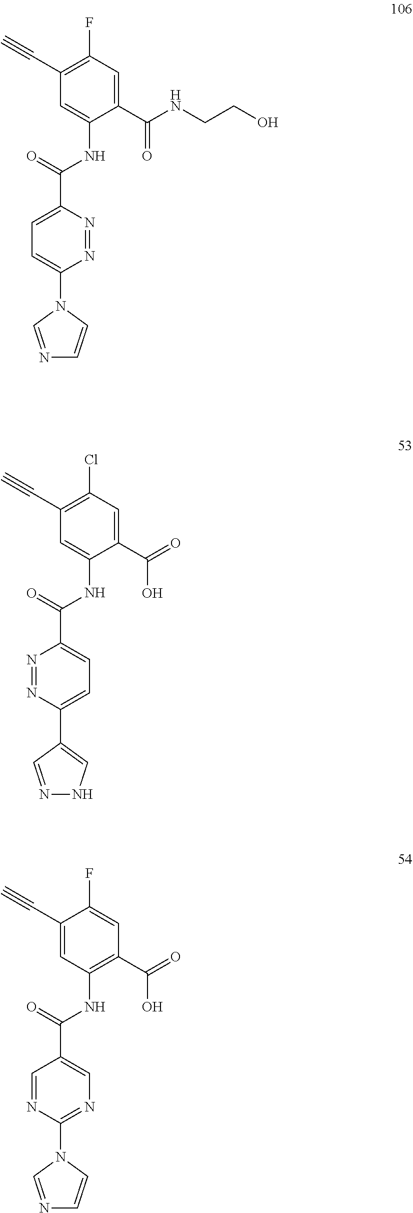 [SMILES: C#Cc1cc(NC(=O)c2ccc(-n3ccnc3)nn2)c(C(=O)NCCO)cc1F ; C#Cc1cc(NC(=O)c2ccc(-c3cn[nH]c3)nn2)c(C(=O)O)cc1Cl ; C#Cc1cc(NC(=O)c2cnc(-n3ccnc3)nc2)c(C(=O)O)cc1F]

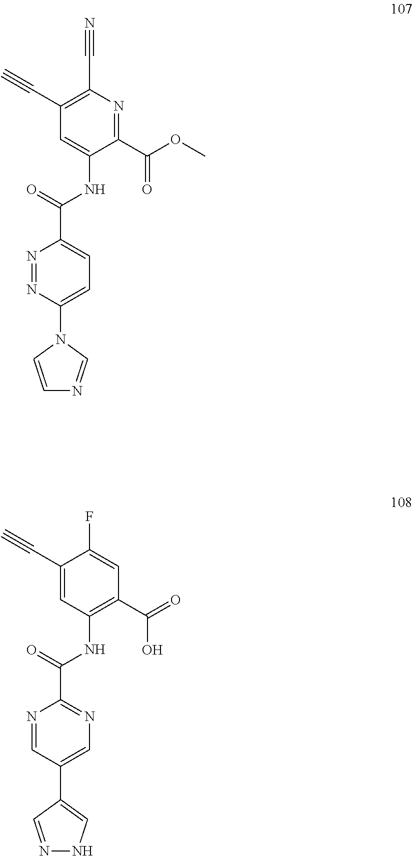

6. The compound according to claim 1, wherein the compound is one selected from the following table:

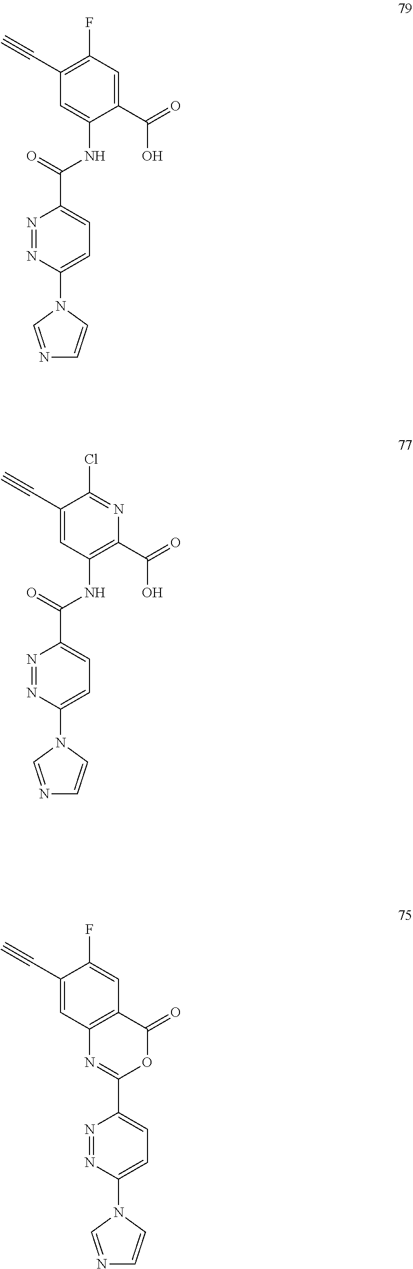 [SMILES: C#Cc1cc(NC(=O)c2ccc(-n3ccnc3)nn2)c(C(=O)O)cc1F ; C#Cc1cc(NC(=O)c2ccc(-n3ccnc3)nn2)c(C(=O)O)nc1Cl ; C#Cc1cc2nc(-c3ccc(-n4ccnc4)nn3)oc(=O)c2cc1F]

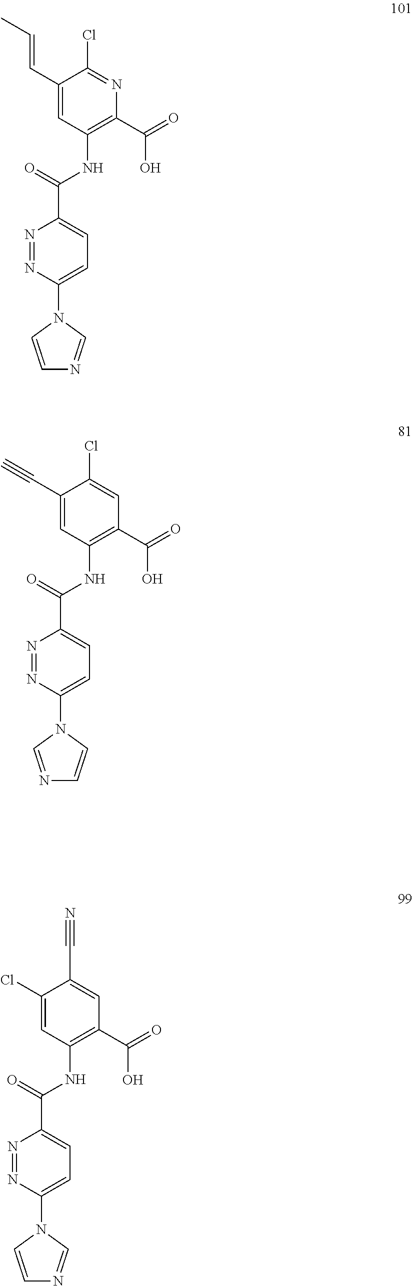 [SMILES: C/C=C/c1cc(NC(=O)c2ccc(-n3ccnc3)nn2)c(C(=O)O)nc1Cl ; C#Cc1cc(NC(=O)c2ccc(-n3ccnc3)nn2)c(C(=O)O)cc1Cl ; N#Cc1cc(C(=O)O)c(NC(=O)c2ccc(-n3ccnc3)nn2)cc1Cl]

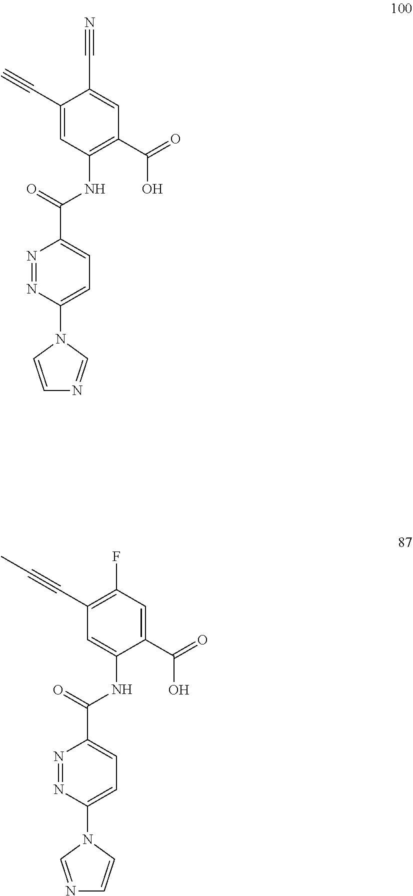 [SMILES: C#Cc1cc(NC(=O)c2ccc(-n3ccnc3)nn2)c(C(=O)O)cc1C#N ; CC#Cc1cc(NC(=O)c2ccc(-n3ccnc3)nn2)c(C(=O)O)cc1F]

7. A method of stimulating expression of interferon genes in a human patient, comprising administering to the patient an effective dose of a compound or pharmaceutically acceptable salt therefore according to claim 1.

8. A method of treating a tumor in a patient, comprising administering to the patient an effective dose of a compound or pharmaceutically acceptable salt therefore according to claim 1.

9. The method according to claim 7, wherein the administering comprises oral or intratumoral administration, or both.

10. The method according to claim 7, wherein administering comprises administering the compound to the patient as an antibody-drug conjugate or in a liposomal formulation.

11. The method according to claim 7, further comprising administering an effective dose of an immune-checkpoint targeting drug.

12. The method according to claim 11, wherein the immune-checkpoint targeting drug comprises an anti-PD-L1 antibody, anti-PD-1 antibody, anti-CTLA-4 antibody, or an anti-4-1BB antibody.

13. The method according to claim 7, further comprising administering ionizing radiation or anticancer drugs.

14. A pharmaceutical composition comprising a compound or pharmaceutically acceptable salt thereof according to claim 1 and a pharmaceutically acceptable carrier.

15. A compound or pharmaceutically acceptable salt thereof according to claim 1 for use in a method of stimulating expression of interferon genes in a human patient.

16. A compound or pharmaceutically acceptable salt thereof according to claim 1 for use in a method of treating a tumor in a patient.

17. The compound for use according to claim 15, wherein the compound is administered to the patient by oral or intratumoral administration, or both.
